(19) United States
(12) Patent Application Publication
(10) Pub. No.: US 2008/0051409 A1
(43) Pub. Date: Feb. 28, 2008

(54) Indolizine Carboxamides and Aza and Diaza Derivatives Thereof

(76) Inventors: Peter Gmeiner, Buckenhof (DE); Harald Hubner, Heroldsbach (DE); Laura Bettinetti, Empoli (FI) (IT); Karin Schlotter, Oettingen (DE)

Correspondence Address: HARNESS, DICKEY, & PIERCE, P.L.C, 7700 BONHOMME, STE 400, ST. LOUIS, MO, 63105, US

(21) Appl. No.: 11/573,145

(22) PCT Filed: Jul. 29, 2005
(86) PCT No.: PCT/EP05/08240

§ 371 Date: Feb. 2, 2007

(30) Foreign Application Priority Data
Aug. 2, 2004 (DE) 10 2004 037 445.7
Aug. 2, 2004 (EP) 04018251.1

(51) Int. Cl. A61K 31/5025; A61K 31/496; A61P 13/02; A61P 15/00; A61P 25/00; C07D 237/26; C07D 295/00

(52) U.S. Cl. 514/252.02; 514/253.04; 544/238; 544/362

(57) ABSTRACT

The present invention concerns neuroreceptor-active carboxamide-substituted indolizine derivatives of general formula I 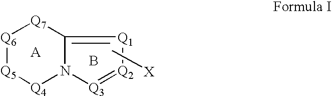
wherein X represents a group of general formula X1

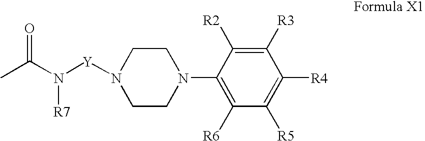

Dopamine is an important neurotransmitter of the central nervous system. Dopamine is effective by bonding to five different dopamine receptors. As a result of their morphology and the nature of their signal transmission these can be classified as D1-like (D1 and D5) and D2-like (D2-, D3- and D4-receptors) (Neve, K. A. The Dopamine Receptors. Humana Press, 1997). The sub-types of the D2 family in particular have an important part to play in the regulation of central nervous processes. While the D2-receptors are predominantly expressed in the basal ganglions and are involved there in the control and modulation of neuromotor circuits, D3-receptors are mainly found in the mesolimbic system, in which emotional and cognitive processes are controlled. Disturbances in the signal transduction of these receptors lead to a number of neuropathological changes which can sometimes result in serious illnesses. As a result the D3-receptor is a promising target for the development of active substances for the treatment of psychiatric illnesses such as schizophrenia or unipolar depressions, of disturbances of consciousness and for treatment of neurodegenerative diseases such as Parkinson's and the dyskineses that can occur in the course of long-term therapy, but also for the treatment of drug dependency (Pulvirenti, L. et al. Trends Pharmacol. Sci. 2002, 23, 151-153, Joyce, J. N. Pharmacol. Ther. 2001, 90, 231-259). Here the most D3-receptor-selective bonding profile should be sought for such active substances. Depending on the intrinsic activity (full agonist, partial agonist, antagonist or inverse agonist) such ligands can have a stimulating, modulating or also inhibiting effect on the pathologically altered dopamine signal transduction system and can thus be used for the treatment of these diseases.

Compounds with an arylpiperazine structure have previously been described as dopamine receptor-active ligands (Robarge, M. J. J. Med. Chem. 2001, 44, 3175-3186). Benzamides and naphthamides with arylpiperazine partial structures are also known as ligands of dopamine receptors (Perrone, R. J. Med. Chem. 1998, 41, 4903-4909; EP 0 779 284 A1). Recently heteroarene amides have also been described as D3-receptor-active compounds (Bettinetti, L. et al. J. Med. Chem. 2002, 45, 4594-4597, Leopoldo, M. et al. J. Med. Chem. 2002, 45, 5727-5735, WO 2004/004729 A1). A phenylpiperazinylnaphthamide has also recently been reported on as a selective D3-partial agonist, which demonstrated hopeful activities in the animal model, and which could be used for the treatment of cocaine addiction (Pilla, M. et al. Nature 1999, 400, 371-375). Furthermore, because of the characteristic features of this compound elimination of the serious motor impairments (dyskinesias) caused by long-term treatment of Parkinson's disease with the pharmaceutical preparations L-DOPA can be achieved (Bezard, E. et al. Nature Med. 2003, 9, 762-767). The most recent literature describes the neuro-protective effect of D3-selective partial agonists against MPTP-induced neurone loss in mice as a murine model for Parkinson's disease (Boeckler, F. et al. Biochem. Pharmacol. 2003, 6, 1025-1032).

Of the range of arylpiperazinylheteroarene carboxamides structure examples with oxygen-, sulphur- or nitrogen-containing heteroarene carboxylic acid components are above all described (ES 2027898; EP 343 961; U.S. Pat. No. 3,646,047; U.S. Pat. No. 3,734,915; WO 2004/024878; Leopoldo, M. et al. J. Med. Chem. 2002, 45, 5727-5735, WO 2004/004729 A1). Indolizine-substituted ligands are not disclosed in these references.

Bettinetti, L. et al. J. Med. Chem. 2002, 45, 4594-4597 described for the first time a few pyrazolo[1,5-a]pyridines with an affinity to the D3-receptor. Other indolizine-substituted ligands have, however, not been described to date.

In connection with our structure-effect research into dopamine receptor ligands we have discovered new compounds of formula (I)-(IX). During in vitro research these demonstrated a particularly high affinity and selective bonding characteristics to the D3-receptor. Some compounds also demonstrate a notable affinity to serotoninergic receptors, in particular to the 5-HT1a-receptor.

The compounds according to the invention could therefore constitute valuable therapeutic agents for the treatment of central nervous system disorders, such as schizophrenia or various types of depression, for neuroprotection in neurodegenerative diseases, in addictive disorders, glaucoma, cognitive disorders, restless leg syndrome, attention deficit hyperactive syndrome (ADHS), hyperprolactinemia, hyperprolactinomia and autism, in idiopathic or medically-induced extrapyramidal motor disturbances, such as acathisia, rigor, dystonias and dyskinesias, as well as various disorders of the urinary tract.

The subject-matter of this invention comprises compounds of the general formula I, 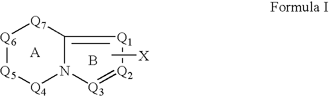
in which:
A is a saturated or aromatic 6-membered ring;
B is an aromatic 5-membered ring; the heteroarene formed from A+B has a total of a maximum of three N-atoms and precisely one X group;
Q1, Q2 and Q3 are in each case and independently of each other N, CH or C—R1;
Q4 is N—R, CH—R1′ or C—R1R1′;
Q5, Q6 and Q7 are independently of each other CH—R1′ or C—R1R1′;
R1 is in each case selected from the hydroxy, alkyl, alkyloxy, alkylthio, alkenyl, alkinyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl and alkylsulfonylamino group;
R1′ is absent if ring A is aromatic or is hydrogen if ring A is saturated;
R is absent if ring A is aromatic or is selected from hydrogen, alkyl, phenyl, alkylcarbonyl, phenylcarbonyl, phenylalkyl and phenylsulfonyl, if ring A is saturated;
X is a group bonded to a C-atom of an aromatic ring A or B of the general formula X1 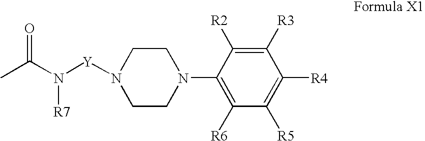
in which:
Y is an unbranched, saturated or unsaturated hydrocarbon chain with 2-5 hydrocarbon atoms or a chain —(CH2)ₒ-Z-(CH2)ₚ, in which Z is selected from the residues cyclopentyl, cyclohexyl and cycloheptyl, wherein o and p in each case and independently of each other have the value 0, 1, 2 or 3 and wherein the sum of o and p is a maximum of 3;
R2, R3, R4, R5 and R6 are in each case selected independently of each other from the hydrogen, hydroxy, alkyl, alkyloxy, alkylthio, alkenyl, alkinyl, phenyl, phenylalkyl, phenoxy, phenylalkyloxy, halogen, trifluoromethyl, alkylcarbonyl, phenylcarbonyl, phenylalkyloxycarhonyl, alkyloxycarbonyl, cyano, nitro, amino, carboxy, sulfo, sulfamoyl, sulfonylamino, alkylaminosulfonyl and alkylsulfonylamino group, wherein two vicinal residues A2, R3, R4, R5 and R6 together with the C-atoms of the phenyl ring to which they are bonded, can form an oxygen-containing 5-, 6- or 7-membered ring;
R7 is hydrogen, alkyl or phenylalkyl;
in the form of the free base, the physiologically acceptable salts and possible enantiomers and diastereomers,

with the proviso of exclusion of
(a) compounds in which the heterocycle is a pyrazolo[1,5-a]pyridine, in particular if this carries as the sole substituent the X group, but no R1 substituent, wherein for X: R2=methoxy; R3, R4, R5, R6 and R7 are in each case hydrogen and
   (i) Y=ethylene, n-propylene or n-butylene or
   (ii) Y=n-pentylene and X is in 2- or 3-position linked with the pyrazolo[1,5-a]pyridine core
(b) The compound N-4-(4-(2-chlorophenyl)piperazin-1-yl)butyl-7-methylpyrazolo[1,5-a]pyridin-3-ylcarbamide.

In the compounds of general formula I, as defined in more detail above, the X group can basically be linked to any ring-forming carbon of an aromatic ring A or B suitable for bonding. If A is a saturated ring, X is bonded to a carbon atom of ring B. The significance of the groups Q1, Q2, Q3, Q4, Q5, Q6 and Q7 in formula I, as described in more detail above, must accordingly be understood according to the invention to be that one of the ring-forming carbons of an aromatic ring contained in the groups Q1, Q2, Q3, Q4, Q5, Q6 and Q7 is substituted with the X group and forms the C—X group.

The term “saturated ring A” and grammatical equivalents of this term mean in the present patent application that the ring A has maximum saturation, i.e. all ring-forming atoms of ring A which are not simultaneously part of aromatic ring B are completely saturated.

In one embodiment of the invention the two rings A and B, apart from the X group, have a maximum of 4, 3, 2 or 1 substituents R1 or are unsubstituted apart from the X group.

In a preferred embodiment of the invention the R1 substituents of the heteroarenes in the compounds according to the invention of general formulae I, II, III, IV, V, VI, VII, VIII and IX are selected from the group comprising hydroxy, fluorine, chlorine, bromine, trifluoromethyl, cyano, amino, carboxy, sulfo, sulfamoyl, unsubstituted or hydroxy substituted C1-C6 alkyl, unsubstituted or hydroxy substituted C1-C6 alkyloxy, unsubstituted or hydroxy substituted C1-C6 alkylthio, unsubstituted C2-C6 alkinyl, unsubstituted or fluorine, chlorine or bromine and/or one or more methoxy groups substituted phenyl, unsubstituted or fluorine, chlorine or bromine and/or one or more methoxy groups substituted phenoxy, —C(O)—C1-C6 alkyl wherein the alkyl is unsubstituted or substituted with hydroxy, —C(O)-phenyl, wherein the phenyl is in each case unsubstituted or substituted with fluorine, chlorine or bromine and/or with one or more methoxy groups, C1-C6 alkyloxycarbonyl, wherein the alkyl is unsubstituted or substituted with hydroxy, C1-6 alkylaminosulfonyl, in particular methylaminosulfonyl and C1-6 alkylsulfonylamino, in particular methanesulfonylamino.

The substituent Q4 in Ring A, depending on the degree of saturation of the ring A, stands for N—R, CH—R1′ or C—R1R1′. In a saturated ring A, R1′ stands for hydrogen and Q4 is selected from NR, CH₂ and CH—R1, wherein R is preferably selected from hydrogen, phenylalkyl and phenylsulfonyl and wherein R1 has the significance defined in more detail above. In an aromatic ring A the substituents R and R1′ are absent; Q4 is then selected from among N, CH and C—R1. If Q4 contains a nitrogen atom, this is preferably uncharged.

R1, R3, R5 and R6 are in the compounds according to the invention of the general formulae I, II, III, IV, V, VI, VII, VIII and IX preferably and independently of each other selected from the group comprising hydroxy, fluorine, chlorine, bromine, trifluoromethyl, cyano, amino, carboxy, sulfo, sulfamoyl, unsubstituted or hydroxy substituted C1-C6 alkyl, unsubstituted or hydroxy substituted C1-C6 alkyloxy, unsubstituted or hydroxy substituted C1-C6 alkylthio, unsubstituted C2-C6 alkinyl, unsubstituted or fluorine, chlorine or bromine and/or one or more methoxy groups substituted phenyl, unsubstituted or fluorine, chlorine or bromine and/or one or more methoxy groups substituted phenoxy, —C(O)—C1-C6 alkyl, wherein the alkyl is unsubstituted or hydroxy substituted, —C(O)-phenyl, phenylalkyloxy or phenylalkyloxycarbonyl, wherein the phenyl is in each case unsubstituted or fluorine, chlorine or bromine and/or one or more methoxy groups substituted, C1-C6 alkyloxycarbonyl, wherein the alkyl is unsubstituted or hydroxy substituted, C1-6 alkylaminosulfonyl, in particular methylaminosulfonyl and C1-6 alkylsulfonylamino, in particular methanesulfonylamino, or two vicinal residues R2, R3, R5 and R6 form together with the C-atoms of the phenyl ring to which they are bonded, an oxygen-containing 5-, 6- or 7-membered ring,

while R4 preferably represents hydrogen.

In a preferred embodiment of the invention Y in the compounds according to the invention is a chain —(CH₂)ₚ-Z-(CH₂)ₒ—, wherein Z is selected from the residues cyclopentyl, cyclohexyl and cycloheptyl, and wherein p and o are independently of each other selected from 0, 1 and 2 and together provide a maximum value of 2 or 1 or are both 0.

In the compounds of general formula I Y is preferably a hydrocarbon chain of formula —(CH2)_{q}- with q=2, 3, 4 or 5, most particularly preferably with n=4 or 5. X thus most particularly preferably represents a group of general formula X2 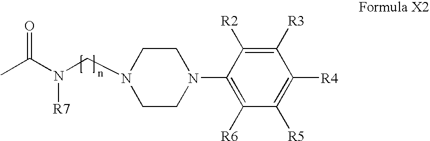
in which n has the value 4 or 5 and the substituents R2, R3, R4, R5, R6 and R7 have the significance described in more detail above.

In one embodiment of the invention at least one of the substituents R2, R3, R5 and R6 is a halogen atom, in particular fluorine or chlorine, while R4 preferably represents hydrogen.

In another preferred embodiment at least one of the two residues R2 and R3 stands for a substituent other than hydrogen, in particular for alkyl, phenyl, alkyloxy, phenylalkyloxy, alkylthio, trifluoromethyl, cyano, a nitro group or a halogen, in particular methyl, methoxy, ethoxy, benzyloxy, methylmercapto, trifluoromethyl, cyano, nitro, fluorine or chlorine, particularly preferably R2 and R3 both being halogens, and most particularly preferably chlorine, while the residues R4, R5 and R6 in compounds according to the invention or in formula X1 and formula X2 stand for hydrogen in each case.

In a preferred embodiment of the invention, in particular if the heterocycle is a pyrazolo[1,5-a]pyridine, one of the two substituents R2 or R3 is selected from alkyl, phenyl, alkyloxy, phenylalkyloxy, alkylthio, trifluoromethyl, cyano, a nitro group or a halogen, in particular methyl, methoxy, ethoxy, benzyloxy, methylmercapto, trifluoromethyl, cyano, nitro, fluorine or chlorine, particularly preferably R2 and R3 both being halogens, and most particularly preferably chlorine.

In a further preferred embodiment of the invention in the compounds of general formula I two vicinal substituents selected from R2, R3, R5 and R6, and in particular the substituents R2 and R3 together with the phenyl residue, with which they are bonded, form a chromane or dihydrobenzofurane, while R4 preferably represents hydrogen.

A preferred embodiment of the invention concerns compounds of general formula I, wherein:
(a) the two rings A and B of the heteroarene have, in addition to the X group, a maximum of 2 substituents R1
(b) R7 is hydrogen
(c) X represents a group of general formula X2 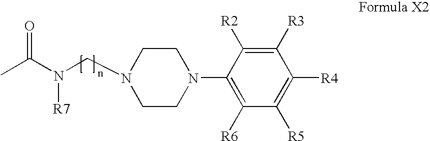
in which n has the value 4 or 5 and
(d) R2, R3, R5 and R6 are preferably and in each case independently of each other selected from the group comprising hydroxy, fluorine, chlorine, bromine, trifluoromethyl, cyano, amino, carboxy, sulfo, sulfamoyl, unsubstituted or hydroxy substituted C1-C6 alkyl, unsubstituted or hydroxy substituted C1-C6 alkyloxy, unsubstituted or hydroxy substituted C1-C6 alkylthio, unsubstituted C2-C6 alkinyl, unsubstituted or fluorine, chlorine or bromine and/or one or more methoxy groups substituted phenoxy, —C(O)—C1-C6 alkyl, wherein the alkyl is unsubstituted or hydroxy substituted, —C(O)-phenyl, phenylalkyl, phenylalkyloxy or phenylalkyloxycarbonyl, wherein the phenyl is in each case unsubstituted or fluorine, chlorine or bromine and/or one or more methoxy groups substituted, C1-C6 alkyloxycarbonyl, wherein the alkyl is unsubstituted or hydroxy substituted, C1-C6 alkylaminosulfonyl, in particular methylaminosulfonyl and C1-6 alkylsulfonylamino, in particular methanesulfonylamino, or two vicinal residues R2, R3, R5 and R6 together with the C-atoms of the phenyl ring with which they are bonded, form an oxygen-containing 5-, 6- or 7-membered ring;
(e) R4 represents hydrogen;
   on condition that, as described in more detail above, certain compounds are excluded as a proviso.

Examples of indolizine derivatives of general formula I according to the invention are: 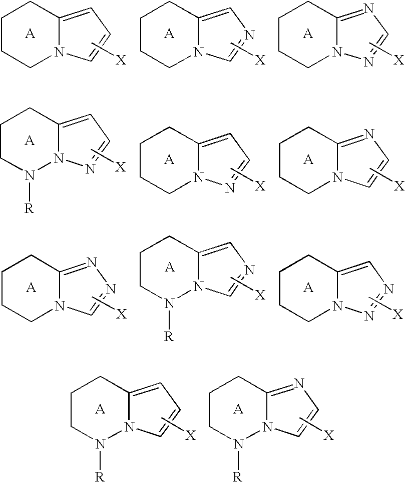
in which:
the ring A is in each case saturated or aromatic;
the ring-forming C-atoms of rings A and B can in each case be substitute d independently of each other with R1;
R, R1 and X have the significance as described in more detail above.

A preferred embodiment of the invention concerns compounds of formula II 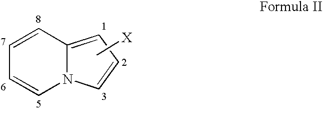
in which:
the indolizine core in positions 1-3 and 5-8, as shown in formula II, apart from the N group can also have one or more, e.g. 1, 2, 3 or 4 further substituents R1, which in each case are selected independently of each other from hydroxy, alkyl, alkyloxy, alkylthio, alkenyl, alkinyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl and alkylsulfonylamino;
X is linked to any position 1-3 or 5-8 of the indolizine and represents a group of general formula X1 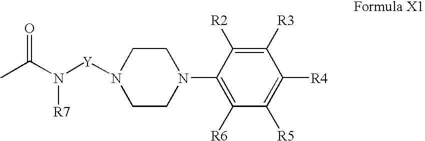
in which:
Y is an unbranched, saturated or unsaturated hydrocarbon chain with 2-5 carbon atoms or a chain —(CH2)ₒ-Z-(CH2)ₚ, in which Z is selected from the residues cyclopentyl, cyclohexyl and cycloheptyl, wherein o and p in each case and independently of each other have the value 0, 1, 2 or 3 and wherein the sum of o and p is a maximum of 3;
R2, R3, R4, R5 and R6 are in each case and independently of each other selected from the group comprising hydrogen, hydroxy, alkyl, alkyloxy, alkylthio, alkenyl, alkinyl, phenyl, phenylalkyl, phenoxy, phenylalkyloxy, halogen, trifluoromethyl, alkylcarbonyl, phenylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino, wherein two vicinal residues R2, R3, R4, R5 and R6 together with the C-atoms of the phenyl ring to which they are bonded, can form an oxygen-containing 5-, 6- or 7-membered ring;
wherein R4 preferably represents hydrogen.

R7 is hydrogen, alkyl or phenylalkyl.

In one embodiment of the invention the heteroarene in formula II is unsubstituted apart from the X group or carries in positions 1 and/or 2 one or more residues R1, as defined in more detail above, in particular cyano or alkyl, e.g. methyl.

The substituent X is preferably linked with the 1, 2 and 3-position of the indolizine (formula II).

In one embodiment of the invention Y in compounds of general formula II is a chain —(CH2)ₒ-Z-(CH2)ₚ, wherein Z is selected from the residues cyclopentyl, cyclohexyl and cycloheptyl and wherein o and p in each case and independently of each other have the value 0, 1 or 2 and preferably both together have a maximum value of 2 or 1 or both are 0.

Y is in the compounds of general formula II preferably a hydrocarbon chain of formula —(CH2)_{q}- with q=2, 3, 4 or 5, most particularly preferably with n=4 or 5. In formula II, therefore, X represents particularly preferably a group of general formula X2 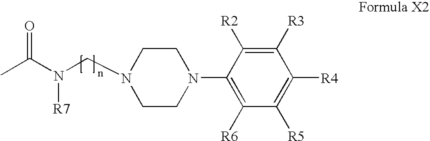
in which n has the value 4 or 5 and the substituents R2, R3, R4, R5, R6 and R7 have the significance described in more detail above.

R7 is preferably hydrogen.

In one embodiment of the invention at least one of the substituent R2, R3, R5 and R6 in the compounds of general formula II is a C1-6 alkyloxy group, e.g. a methoxy or a halogen atom, in particular fluorine or chlorine, while R4 preferably represents hydrogen.

In another preferred embodiment at least one of the two residues R2 and R3 in the compounds of general formula II stands for a substituent other than hydrogen, in particular for halogen or C1-6 alkyloxy, while the residues R4, R5 and R6 in formula II in each case stand for hydrogen.

In a preferred embodiment of the invention one of the two substituents R2 or R3 in the compounds of general formula II is a C1-6 alkyloxy group, in particular methoxy, or a halogen, in particular fluorine or chlorine, particularly preferably R2 and R3 both being halogen, most particularly preferably chlorine.

In a further preferred embodiment of the invention, in the compounds of general formula II two vicinal substituents selected from R2, R3, R5 and R6, and in particular the substituents R2 and R3, together with the phenyl residue, to which they are bonded, form a chromane or dihydrobenzofurane, while R4 preferably represents hydrogen.

Another preferred embodiment of the invention concerns compounds of formula III 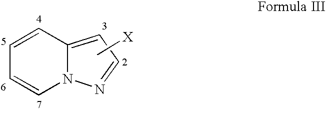
in which:
the pyrazolo[1,5-a]pyridine core can in positions 2-7, as shown in formula III, apart from the X group, also carry one or more, e.g. 1, 2, 3 or 4 further substituents R1, which are in each case selected independently of each other from hydroxy, alkyl, alkyloxy, alkylthio, alkenyl, alkinyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl and alkylsulfonylamino;
X is linked with any position 2-7 of the pyrazolo[1,5-a]pyridine and represents a group of general formula X1 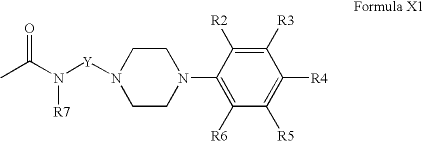
in which:
Y is an unbranched, saturated or unsaturated hydrocarbon chain with 2-5 carbon atoms or a chain —(CH2)ₒ-Z-(CH2)ₚ, in which Z is selected from the residues cyclopentyl, cyclohexyl and cycloheptyl, wherein o and p in each case and independently of each other have the value 0, 1, 2 or 3 and wherein the sum of o and p is a maximum of 3;
R2, R3, R4, R5 and R6 are in each case and independently of each other selected from the group comprising hydrogen, hydroxy, alkyl, alkyloxy, alkylthio, alkenyl, alkinyl, phenyl, phenylalkyl, phenoxy, phenylalkyloxy, halogen, trifluoromethyl, alkylcarbonyl, phenylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino, wherein two vicinal residues R2, R3, R4, R5 and R6 together with the C-atoms of the phenyl ring, to which these are bonded, can form an oxygen-containing 5-, 6- or 7-membered ring, wherein R4 preferably represents hydrogen;
R7 is hydrogen, alkyl or phenylalkyl.

The X group is preferably bonded with positions 2, 5 or 6 of the pyrazolo[1,5-a]pyridine of formula III.

In one embodiment the pyrazolo[1,5-a]-pyridine core is substituted in at least one of positions 5 or 6. In a preferred embodiment of the invention the pyrazolo[1,5-a]pyridine carries in position 5 a methoxy- or CF₃-residue and/or in position 6 a halogen atom, in particular if X is bonded to position 2 of the heteroarene.

In another preferred embodiment the pyrazolo[1,5-a]-pyridine core in the compounds of general formula III apart from the mandatory substituent X is unsubstituted, in particular if X is bonded to positions 5 or 6 of the heteroarene.

Y is in the compounds of general formula III preferably a hydrocarbon chain of formula —(CH2)_{q}- with q=2, 3, 4 or 5, with quite particular preference with n=4 or 5. In formula III the X group therefore represents particularly preferably a group of general formula X2 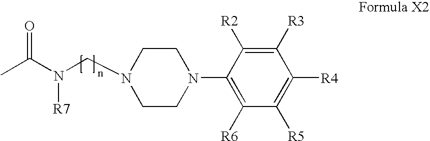
in which n has the value 4 or 5 and the substituents R2, R3, R4, R5, R6 and R7 have the significance described in more detail above.

R7 is preferably hydrogen.

In one embodiment of the invention at least one of the substituents R2, R3, R5 and R6 in the compounds of general formula III is an alkyl (in particular methyl), phenyl, alkyloxy (in particular methyloxy and ethyloxy), phenylalkyloxy (in particular phenyloxy), alkylthio (in particular methylthio), trifluoromethyl, cyano or a nitro group or a halogen atom, in particular fluorine or chlorine, while R4 preferably represents hydrogen.

In another preferred embodiment at least one of the two residues R2 and R3 in the compounds of general formula III stands for a substituent other than hydrogen, in particular for halogen, alkyl (in particular methyl), phenyl, alkyloxy (in particular methyloxy and ethyloxy), phenylalkyloxy (in particular benzyloxy), alkylthio (in particular methylthio), trifluoromethyl, cyano or nitro, while residues R4, R5 and R6 in each case stand for hydrogen.

In a preferred embodiment of the invention R4 is hydrogen and one of the two substituents R2 or R3 in the compounds of general formula III is a halogen, alkyl (in particular methyl), phenyl, alkyloxy (in particular methyloxy and ethyloxy), phenylalkyloxy (in particular benzyloxy), alkylthio (in particular methylthio), trifluoromethyl, cyano or nitro, in particular fluorine or chlorine, particularly preferably R2 and R3 are both halogen or alkyl, most particularly preferably chlorine or methyl.

In one embodiment of the invention R2 in the compounds of general formula III stands for a C1-6 alkyloxy group, in particular for methoxy, provided that
(a) at least one of the substituents R3, R5, R6 and R7 represents a residue other than hydrogen and/or
(b) the pyrazolo[1,5-a]pyridine core is substituted with at least one substituent R1.

In another embodiment of the invention R2 is not a methoxy. In another embodiment of the invention R2 in the compounds of general formula III is not an alkyloxy.

In a further embodiment of the invention in the compounds of general formula III two vicinal substituents selected from R2, R3, R5 and R6, and in particular substituents R1 and R3 together with the phenyl residue to which they are bonded, form a chromane or dihydrobenzofurane.

A further preferred embodiment of the invention comprises compounds of general formula IV, 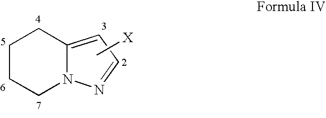
in which:
the tetrahydropyrazolo[1,5-a]pyridine core can in positions 2-7 as shown in formula IV, apart from the X group, also carry one or more, e.g. 1, 2, 3 or 4 additional substituents R1, which are in each case selected independently of each other from hydroxy, alkyl, alkyloxy, alkylthio, alkenyl, alkinyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl and alkylsulfonylamino;
X is preferably linked to position 2 or 3 of the tetrahydropyrazolo[1,5-a]pyridine and represents a group of general formula X1 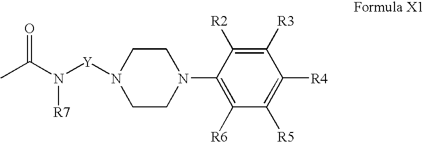
in which:
Y is an unbranched, saturated or unsaturated hydrocarbon chain with 2-5 carbon atoms or a chain —(CH2)ₒ-Z-(CH2)ₚ, in which Z is selected from the residues cyclopentyl, cyclohexyl and cycloheptyl, wherein o and p in each case and independently of each other have the value 0, 1, 2 or 3 and wherein the sum of o and p is a maximum of 3;
R2, R3, R4, R5 and R6 are in each case and independently of each other selected from the group comprising hydrogen, hydroxy, alkyl, alkyloxy, alkylthio, alkenyl, alkinyl, phenyl, phenylalkyl, phenoxy, phenylalkyloxy, halogen, trifluoromethyl, alkylcarbonyl, phenylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino, wherein two vicinal residues R2, R3, R4, R5 and R6 together with the C-atoms of the phenyl ring to which they are bonded, can form an oxygen-containing 5-, 6- or 7-membered ring, wherein R4 preferably represents hydrogen;
R7 is hydrogen, alkyl or phenylalkyl.

In one embodiment of the invention the heteroarene in formula IV is unsubstituted as far as the X group or carries in positions 5 and/or 6 one or more residues R1, as defined in more detail above, in particular alkyl, e.g. methyl.

Y in the compounds of general formula IV is preferably a hydrocarbon chain of formula —(CH2)_{q}- with q=2, 3, 4 or 5, with quite particular preference with n=4 or 5. Most particularly preferably therefore in formula IV, X represents a group of general formula X2 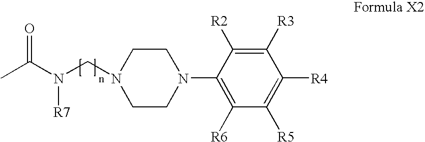
in which n has the value 4 or 5 and the substituents X2, R3, R4, R5, R6 and R7 have the significance described in more detail above.

R7 is preferably hydrogen.

In one embodiment of the invention at least one of the substituents R2, R3, R5 and R6 in the compounds of general formula IV is a C1-6 alkyloxy group, in particular methoxy, or a halogen atom, in particular fluorine or chlorine.

In another preferred embodiment at least one of the two residues R2 and R3 in the compounds of general formula IV stands for a substituent other than hydrogen, in particular for halogen or C1-C6 alkyloxy, while the residues R4, R5 and R6 in each case stand for hydrogen.

In a preferred embodiment of the invention one of the two substituents R2 or R3 in the compounds of general formula IV is a C1-6 alkyloxy group, in particular methoxy or halogen, in particular fluorine or chlorine, particularly preferably R2 and R3 are both halogen, with quite particular preference chlorine, while R4 preferably represents hydrogen.

In a further embodiment of the invention in the compounds of general formula IV two vicinal substituents selected from R2, R3, R5 and R6, and in particular the substituents R2 and R3 together with the phenyl residue, to which they are bonded, form a chromane or dihydrobenzofurane, while R4 preferably represents hydrogen.

Another preferred embodiment of the invention concerns compounds of formula V 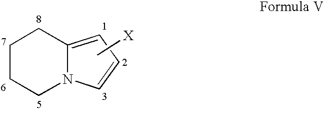
in which:
the tetrahydroindolizine core can in positions 1-3 and 5-8, as shown in formula V, apart from the X group, also carry one or more, e.g. 1, 2, 3 or 4 additional substituents R1, which in each case are selected independently of each other from hydroxy, alkyl, alkyloxy, alkylthio, alkenyl, alkinyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl and alkylsulfonylamino;
X is linked with any position 1-3 of the tetrahydroindolizine and represents a group of general formula X1 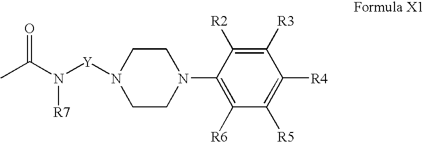
in which:
Y is an unbranched, saturated or unsaturated hydrocarbon chain with 2-5 carbon atoms or a chain —(CH2)ₒ-Z-(CH2)ₚ, in which Z is selected from the residues cyclopentyl, cyclohexyl and cycloheptyl, wherein o and p in each case and independently of each other have the value 0, 1, 2 or 3 and wherein the sum of o and p is a maximum of 3;
R2, R3, R4, R5 and R6 are in each case and independently of each other selected from the group comprising hydrogen, hydroxy, alkyl, alkyloxy, alkylthio, alkenyl, alkinyl, phenyl, phenylalkyl, phenoxy, phenylalkyloxy, halogen, trifluoromethyl, alkylcarbonyl, phenylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino, wherein two vicinal residues R2, R3, R4, R5 and R6 together with the C-atoms of the phenyl ring to which they are bonded, can form an oxygen-containing 5-, 6- or 7-membered ring, wherein R4 preferably represents hydrogen;
R7 is hydrogen, alkyl or phenylalkyl.

In one embodiment of the invention the heteroarene in formula V is unsubstituted as far as the X group.

The substituent X is preferably linked with the 1, 2 and 3-positions of the tetrahydroindolizine (formula V) and particularly preferably with position 2.

Y is in the compounds of general formula V preferably a hydrocarbon chain of formula —(CH2)_{q}- with q=2, 3, 4 or 5, with quite particular preference with n=4 or 5. X thus represents in formula V particularly preferably a group of general formula X2 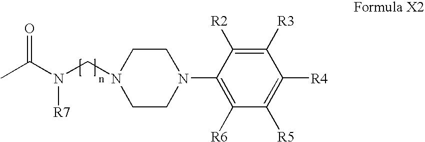
in which n has the value 4 or 5 and the substituents R2, R3, R4, R5, R6 and R7 have the significance described in more detail above.

R7 is preferably hydrogen.

In one embodiment of the invention at least one of the substituents R2, R3, R5 and R6 in the compounds of general formula V is a C1-6 alkyloxy group, e.g. a methoxy or a halogen atom, in particular fluorine or chlorine, while R4 preferably represents hydrogen.

Another preferred embodiment of the invention concerns compounds of formula VI 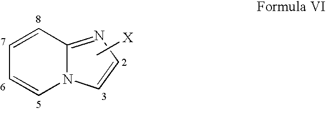
in which:
the heteroarene core can in positions 2-3 and 5-8, as shown in formula VI, apart from the X group, also carry one or more, e.g. 1, 2, 3 or 4 additional substituents R1 which are in each case selected independently of each other from hydroxy, alkyl, alkyloxy, alkylthio, alkenyl, alkinyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl and alkylsulfonylamino;
X is linked with any position 2-3 or 5-8 of the heteroarene and represents a group of general formula X1 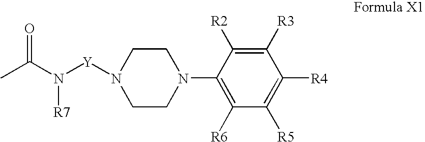
in which:
Y is an unbranched, saturated or unsaturated hydrocarbon chain with 2-5 carbon atoms or a chain —(CH2)ₒ-Z-(CH2)ₚ, in which Z is selected from the residues cyclopentyl, cyclohexyl and cycloheptyl, wherein o and p in each case and independently of each other have the value 0, 1, 2 or 3 and wherein the sum of o and p is a maximum of 3;
R2, R3, R4, R5 and R6 are in each case and independently of each other selected from the group comprising hydrogen, hydroxy, alkyl, alkyloxy, alkylthio, alkenyl, alkinyl, phenyl, phenylalkyl, phenoxy, phenylalkyloxy, halogen, trifluoromethyl, alkylcarbonyl, phenylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino, wherein two vicinal residues R2, R3, R4, R5 and R6 together with the C-atoms of the phenyl ring to which they are bonded, can form an oxygen-containing 5-, 6- or 7-membered ring, wherein R4 preferably represents hydrogen;
R7 is hydrogen, alkyl or phenylalkyl.

In one embodiment of the invention the heteroarene in formula VI is unsubstituted apart from the X group or carries in the 2- or 6-position a residue R1 as defined in more detail above, in particular alkyl, e.g. methyl, or halogen.

The substituent X is preferably linked with the 2, 3 or 6-position of the heteroarene (formula VI).

Y is in the compounds of general formula VI preferably a hydrocarbon chain of formula (CH2)_{q}- with q=2, 3, 4 or 5, most particularly preferably with n=4 or 5. X thus represents in formula VI particularly preferably a group of general formula X2 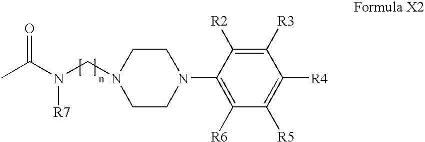
in which n has the value 4 or 5 and the substituents R2, R3, R4, R5, R6 and R7 have the significance described in more detail above.

R7 is preferably hydrogen.

In one embodiment of the invention at least one of the substituents R2, R3, R5 and R6 in the compounds of general formula VI is a methoxy group or a halogen atom, in particular fluorine or chlorine, while R4 preferably represents hydrogen.

Another preferred embodiment of the invention concerns compounds of formula VII 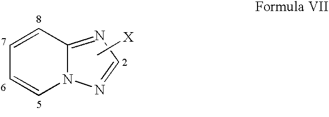
in which:
the heteroarene core can in positions 2 and 5-8, as shown in formula VII, apart from the X group, also carry one or more, e.g. 1, 2, 3 or 4 additional substituents R1, which are in each case selected independently of each other from hydroxy, alkyl, alkyloxy, alkylthio, alkenyl, alkinyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl and alkylsulfonylamino;
X is linked with any position 2 or 5-8 of the heteroarene and represents a group of general formula X1 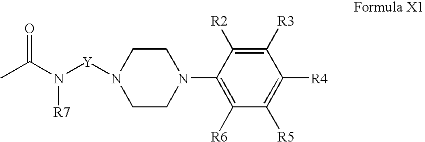
in which:
Y is an unbranched, saturated or unsaturated hydrocarbon chain with 2-5 carbon atoms or a chain —(CH2)ₒ-Z-(CH2)ₚ, in which Z is selected from the residues cyclopentyl, cyclohexyl and cycloheptyl, wherein o and p in each case and independently of each other have the value 0, 1, 2 or 3 and wherein the sum of o and p is a maximum of 3;
R2, R3, R4, R5 and R6 are in each case and independently of each other selected from the group comprising hydrogen, hydroxy, alkyl, alkyloxy, alkylthio, alkenyl, alkinyl, phenyl, phenylalkyl, phenoxy, phenylalkyloxy, halogen, trifluoromethyl, alkylcarbonyl, phenylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino, wherein two vicinal residues R2, R3, R4, R5 and R6 together with the C-atoms of the phenyl ring to which they are bonded, can form an oxygen-containing 5-, 6- or 7-membered ring, wherein R4 preferably represents hydrogen;

R7 is hydrogen, alkyl or phenylalkyl.

In one embodiment of the invention the heteroarene in formula VII is unsubstituted as far as the X group.

The substituent X is preferably linked to the 2-position of the heteroarene (formula VII).

Y is in the compounds of general formula VII preferably a hydrocarbon chain of formula —(CH2)_{q}- with q=2, 3, 4 or 5, most particularly preferably with n=4 or 5. X thus represents in formula VII particularly preferably a group of general formula X2 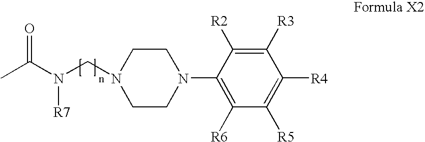
in which n has the value 4 or 5 and the substituents R2, R3, R4, R5, R6 and R7 have the significance described in more detail above.

R7 is preferably hydrogen.

In one embodiment of the invention at least one of the substituents R2, R3, R5 and R6 in the compounds of general formula VII is a halogen atom, in particular fluorine or chlorine, while R4 preferably represents hydrogen.

A preferred embodiment of the invention concerns compounds of formula VIII 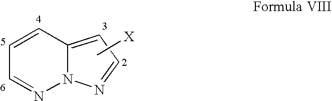
in which:
the heteroarene core can in positions 2-6, as shown in formula VIII, apart from the X group, also carry one or more, e.g. 1, 2, 3 or 4 additional substituents R1, which are in each case selected independently of each other from hydroxy, alkyl, alkyloxy, alkylthio, alkenyl, alkinyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl and alkylsulfonylamino;
X is linked with any position 2-6 of the heteroarene and represents a group of general formula X1 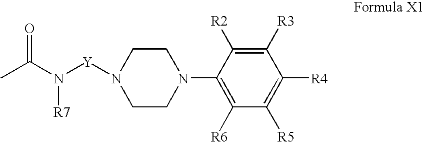
in which:
Y is an unbranched, saturated or unsaturated hydrocarbon chain with 2-5 carbon atoms or a chain —(CH2)ₒ-Z-(CH2)ₚ, in which Z is selected from the residues cyclopentyl, cyclohexyl and cycloheptyl, wherein o and p in each case and independently of each other have the value 0, 1, 2 or 3 and wherein the sum of o and p is a maximum of 3;
R2, R3, R4, R5 and R6 are in each case and independently of each other selected from the group comprising hydrogen, hydroxy, alkyl, alkyloxy, alkylthio, alkenyl, alkinyl, phenyl, phenylalkyl, phenoxy, phenylalkyloxy, halogen, trifluoromethyl, alkylcarbonyl, phenylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino, wherein two vicinal residues R2, R3, R4, R5 and R6 together with the C-atoms of the phenyl ring to which they are bonded, can form an oxygen-containing 5-, 6- or 7-membered ring, wherein R4 preferably represents hydrogen;
R7 is hydrogen, alkyl or phenylalkyl.

In one embodiment of the invention the heteroarene in formula VIII is unsubstituted as far as the X group.

The substituent X is preferably linked to the 2-position of the heteroarene (formula VIII).

Y is in the compounds of general formula VIII preferably a hydrocarbon chain of formula —(CH2)_{q}- with q=2, 3, 4 or 5, most particularly preferably with n=4 or 5. X thus represents in formula VIII particularly preferably a group of general formula X2 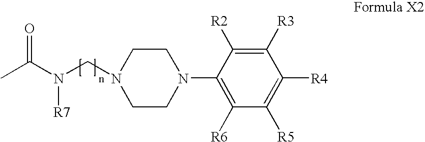
in which n has the value 4 or 5 and the substituents R2, R3, R4, R5, R6 and R7 have the significance described in more detail above.

R7 is preferably hydrogen.

In one embodiment of the invention at least one of the substituents R2, R3, R5 and R6 in the compounds of general formula VIII is a halogen atom, in particular fluorine or chlorine, while R4 preferably represents hydrogen.

A preferred embodiment of the invention concerns compounds of formula IX 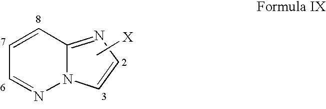
in which:
the heteroarene core can in positions 2-3 and 6-8, as shown in formula IX, apart from the X group, also carry one or more, e.g. 1, 2, 3 or 4 additional substituents R1, which are in each case selected independently of each other from hydroxy, alkyl, alkyloxy, alkylthio, alkenyl, alkinyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl and alkylsulfonylamino;
X is linked with any position 2-3 or 6-8 of the heteroarene and represents a group of general formula X1 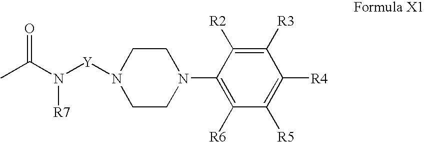
in which:
Y is an unbranched, saturated or unsaturated hydrocarbon chain with 2-5 carbon atoms or a chain —(CH2)ₒ-Z-(CH2)ₚ, in which Z is selected from the residues cyclopentyl, cyclohexyl and cycloheptyl, wherein o and p in each case and independently of each other have the value 0, 1, 2 or 3 and wherein the sum of o and p is a maximum of 3;
R2, R3, R4, R5 and R6 are in each case and independently of each other selected from the group comprising hydrogen, hydroxy, alkyl, alkyloxy, alkylthio, alkenyl, alkinyl, phenyl, phenylalkyl, phenoxy, phenylalkyloxy, halogen, trifluoromethyl, alkylcarbonyl, phenylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino, wherein two vicinal residues R2, R3, R4, R5 and R6 together with the C-atoms of the phenyl ring to which they are bonded, can form an oxygen-containing 5-, 6- or 7-membered ring, wherein R4 preferably represents hydrogen;
R7 is hydrogen, alkyl or phenylalkyl.

In one embodiment of the invention the heteroarene in formula IX is unsubstituted as far as the X group or carries in position 2 and/or position 6 a residue R1 as defined in more detail above, in particular phenyl or halogen.

The substituent X is preferably linked to the 2- or 3-position of the heteroarene (formula IX).

Y is in the compounds of general formula IX preferably a hydrocarbon chain of formula —(CH₂)_{q}— with q=2, 3, 4 or 5, most particularly preferably with n=4 or 5. X thus represents in formula IX particularly preferably a group of general formula X2 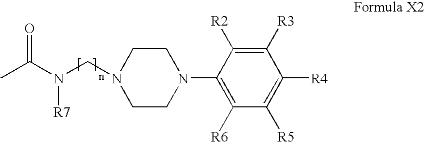
in which n has the value 4 or 5 and the substituents R2, R3, R4, R5, R6 and R7 have the significance described in more detail above.

R7 is preferably hydrogen.

In one embodiment of the invention at least one of the substituents R2, R3, R5 and R6 in the compounds of general formula IX is a methoxy residue or a halogen atom, in particular fluorine or chlorine, while R4 preferably represents hydrogen.

The invention also concerns physiologically acceptable salts of the compounds according to the invention. Examples of such salts are described in the following definitions.

The person skilled in the art will also realise that depending on the choice of substituents geometrical isomers and/or optically active compounds can result. In this case both the isomers and racemates and also the respective pure enantiomeric or possibly diastereomeric forms are the subject-matter of the present invention.

The substituents mentioned in the description and in the attached claims include in particular the following groups.

“Alkyl” can be a branched or unbranched alkyl group, which preferably has between 1 and 10 C-atoms, particularly preferably between 1 and 6 C-atoms (“C1-C6 alkyl”) and most particularly preferably 1, 2 or 3 C-atoms. “C1-C6 alkyl” includes, for example, methyl, ethyl, n-propyl, iso-propyl, n-butyl, iso-butyl, s-butyl, t-butyl, n-pentyl, iso-pentyl, neopentyl, t-pentyl, 1-methylbutyl, 2-methylbutyl, 1-ethylpropyl, 1,2-dimethylpropyl and n-hexyl.

“Alkyl” can also be cyclical or contain a cyclical component, wherein cycles with 3-7 C-atoms are preferred, e.g. cyclopropyl, cyclobutyl, cyclopentyl, cyclohexyl or cycloheptyl. “Alkyl” is preferably not cyclical and contains no cyclical component. Alkyl groups can also be substituted with one or more substituents, in particular with hydroxy or amine. “Alkyl” is preferable unsubstituted or substituted with hydroxy.

“Alkenyl” and “alkinyl” have at least one double or triple bond. They can be branched or unbranched and preferably have between 2 and 6 C-atoms. Alkenyls or alkinyls are preferably bonded to the heteroarene- or phenyl ring of the scaffold of the compound in such a way that the double or triple bond is conjugated with the aromatic ring. Alkenyl and alkinyl can also be substituted with one or more substituents, preferably with phenyl, wherein the phenyl group then is preferably located at C-atom 2 (if the alkenyl or alkinyl is bonded via C-atom 1 to the heteroarene- or phenyl ring of the scaffold). The alkenyls or alkinyls are preferably unsubstituted.

“Alkyloxy” is the —O-alkyl group, in which the alkyl is preferably selected from the groups specified above for “alkyl”. “Alkyloxy” is preferably a C1-C6-alkyloxy group, particularly preferably methoxy.

“Alkylthio” can also be referred to as “alkylmercapto” and is the —S-alkyl group, in which alkyl is preferably selected from the groups specified for “alkyl” above. “Alkylthio” is preferably a C1-C6-alkyl-5-group.

“Alkylaminosulfonyl” includes the —SO₂—NH-alkyl and —SO₂—N-dialkyl groups, in which alkyl is preferably selected from the groups specified above for “alkyl”. “Alkyl” in the “alkylaminosulfonyl” is preferably a C1-C6-alkyl group. “Alkylaminosulfonyl” examples include methylaminosulfonyl, N,N-dimethylaminosulfonyl and butylaminosulfonyl.

“Alkylsulfonylamino” is the —NH—SO₂-alkyl group, in which alkyl is preferably selected from the groups specified above for “alkyl”. “Alkylsulfonylamino” is preferably a C1-C6-alkylsulfonylamino group, e.g. methanesulfonylamino.

“Phenyl” is preferably unsubstituted, but can if necessary be independently substituted one or more times, e.g. with alkoxy, alkyl, trifluoromethyl or halogen.

“Phenylalkyl” is the -alkyl-phenyl group, wherein phenyl and alkyl have the significance as defined above. Phenyl alkyl includes for example phenylethyl and benzyl and is preferably benzyl.

“Phenoxy” is the —O-phenyl group, in which phenyl has the significance defined in more detail above.

“Phenylalkyloxy” is the phenylalkyl-O— group, in which phenylalkyl has the significance defined in more detail above.

“Alkylcarbonyl” includes the —C(O)-alkyl group, in which alkyl is preferably selected from the groups specified above for “alkyl”, and is particularly preferably —C(O)—C1-C6-alkyl.

“Alkylcarbonyl” is preferably acetyl, propionyl or butyryl.

“Phenylcarbonyl” is —C(O)-phenyl, in which phenyl has the significance as defined in more detail above.

“Alkyloxycarbonyl” is the —C(O)—O-alkyl group, in which alkyl is preferably selected from the groups specified above for “alkyl”. “Alkoxycarbonyl” is preferably a (C1-C6-alkyl)oxycarbonyl group.

“Phenylalkyloxycarbonyl” is the phenylalkyl-O—C(O)— group, in which phenylalkyl has the significance defined in more detail above.

“Halogen” includes fluorine, chlorine, bromine and iodine, and is preferably fluorine, chlorine or bromine.

“Sulfamoyl” includes the —SO₂—NH₂ group.

“Sulfonylamino” includes the —NH—SO₂H group.

“Physiologically acceptable salts” include non-toxic addition salts of a base, in particular a compound of formulae (I) to (IV) in the form of the free base, with organic or inorganic acids. Examples of inorganic acids include HCl, HBr, sulphuric acid and phosphoric acid. Organic acids include acetic acid, propionic acid, pyruvic acid, butyric acid, α-, β- or γ-hydroxbutyric acid, valeric acid, hydroxyvaleric acid, caproic acid, hydroxycaproic acid, caprylic acid, capric acid, lauric acid, myristic acid, palmitic acid, stearic acid, glycolic acid, lactic acid, D-glucuronic acid, L-glucoronic acid, D-galacturonic acid, glycine, benzoic acid, hydroxybenzoic acid, gallic acid, salicylic acid, vanillic acid, coumarinic acid, caffeic acid, hippuric acid, orotic acid, L-tartaric acid, D-tartaric acid, D,L-tartaric acid, meso-tartaric acid, fumaric acid, L-malic acid, D-malic acid, D,L-malic acid, oxalic acid, malonic acid, succinic acid, maleic acid, oxalic acid, glutaric acid, hydroxyglutaric acid, ketoglutaric acid, adipinic acid, ketoadipinic acid, pimelic acid, glutamic acid, aspartic acid, phthalic acid, propanetricarboxylic acid, citric acid, isocitric acid, methane sulfonic acid, toluene sulfonic acid, benzene sulfonic acid, camphor sulfonic acid, embonic acid and trifluoromethane sulfonic acid.

The following compounds represent specific embodiments of the compounds according to the invention:
(B69): N-4-(4-(2-methoxyphenyl)piperazin-1-yl)butylindolizin-1-ylcarbamide
(B1): N-4-(4-(2-methoxyphenyl)piperazin-1-yl)butylindolizin-2-ylcarbamide
(B2); N-4-(4-(3-chloro-2-methoxyphenyl)piperazin-1-yl)butylindolizin-2-ylcarbamide
(B3): N-4-(4-(2,3-dichlorophenyl)piperazin-1-yl)butylindolizin-2-ylcarbamide
(B4): N-4-(4-(2,3-difluorophenyl)piperazin-1-yl)butylindolizin-2-ylcarbamide
(B5): N-4-(4-(2,3-dihydrobenzofuran-7-yl)piperazin-1-yl)butylindolizin-2-ylcarbamide
(B49): N-4-(4-(chroman-8-yl)piperazin-1-yl)butylindolizin-2-ylcarbamide
(B70): N-4-(4-(2-methoxyphenyl)piperazin-1-yl)butyl-5,6,7,8-tetrahydroindolizin-2-ylcarbamide
(B71): N-4-(4-(2,3-dichlorophenyl)piperazin-1-yl)butyl-5,6,7,8-tetrahydroindolizin-2-ylcarbamide
(B72): N-4-(4-(2-methoxyphenyl)piperazin-1-yl)butyl-1-cyano-2-methylindolizin-3-ylcarbamide
(B6): N-2-(4-(2,3-dichlorophenyl)piperazin-1-yl)ethylpyrazolo[1,5-a]pyridin-2-ylcarbamide
(B 7): N-3-(4-(2,3-dichlorophenyl)piperazin-1-yl)propylpyrazolo[1,5-a]pyridin-2-ylcarbamide
(B73): N-4-(4-phenylpiperazin-1-yltbutylpyrazolo[1,5-a]pyridin-2-ylcarbamide
(B74): N-4-(4-(2-methylphenyl)piperazin-1-yl)butylpyrazolo[1,5-a]pyridin-2-ylcarbamide
(B75): N-4-(4-(2-biphenyl)piperazin-1-yl)butylpyrazolo[1,5-a]pyridin-2-ylcarbamide
(B76): N-4-(4-(2-ethoxyphenyl)piperazin-1-yl)butylpyrazolo[1,5-a]pyridin-2-ylcarbamide
(B77): N-4-(4-(2-benzyloxyphenyl)piperazin-1-yl)butylpyrazolo[1,5-a]pyridin-2-ylcarbamide
(B78): N-4-(4-(2-methylmercaptophenyl)piperazin-1-yl)butylpyrazolo[1,5-a]pyridin-2-ylcarbamide
(B79): N-4-(4-(2-fluorphenyl)piperazin-1-yl)butylpyrazolo[1,5-a]pyridin-2-ylcarbamide
(B80): N-4-(4-(2-trifluoromethylphenyl)piperazin-1-yl)butylpyrazolo[1,5-a]pyridin-2-ylcarbamide
(B81): N-4-(4-(2-cyanophenyl)piperazin-1-yl)butylpyrazolo[1,5-a]pyridin-2-ylcarbamide
(B82): N-4-(4-(2-nitrophenyl)piperazin-1-yl)butylpyrazolo[1,5-a]pyridin-2-ylarbamide
(B83): N-4-(4-(4-methoxyphenyl)piperazin-1-yl)butylpyrazolo[1,5-a]pyridin-2-ylcarbamide
(B 84): N-4-(4-(3-chloro-2-methoxyphenyl)piperazin-1-yl)butylpyrazolo[1,5-a]pyridin-2-ylcarbamide
(B8): N-4-(4-(2,3-dichlorophenyl)piperazin-1-yl)butylpyrazolo[1,5-a]pyridin-2-ylcarbamide
(B85): N-4-(4-(2,3-dimethylphenyl)piperazin-1-yl)butylpyrazolo[1,5-a]pyridin-2-ylcarbamide
(B86): N-4-(4-(2,3-dihydrobenzofuran-7-yl)piperazin-1-yl)butylpyrazolo[1,5-a]pyridin-2-ylcarbamide
(B87): N-4-(4-(chroman-8-ylpiperazin-1-yl)butylpyrazolo[1,5-a]pyridin-2-ylcarbamide
(B88): N-4-(4-(2,4-dimethoxyphenyl)piperazin-1-yl)butylpyrazolo[1,5-a]pyridin-2-ylcarbamide
(B10): N-4-(4-(2-methoxyphenyl)piperazin-1-yl)butyl-3-bromopyrazolo[1,5-a]pyridin-2-ylcarbamide
(B11): N-4-(4-(2-methoxyphenyl)piperazin-1-yl)butyl-3-chloropyrazolo[1,5-a]pyridin-2-ylcarbamide
(B50): N-4-(4-(2,3-dichlorophenyl)piperazin-1-yl)butyl-5-methoxypyrazolo[1,5-a]pyridin-2-ylcarbamide
(B51): N-4-(4-(2-methoxyphenyl)piperazin-1-yl)butyl-5-methoxypyrazolo[1,5-a]pyridin-2-ylcarbamide
(B12): N-4-(4-(2-methoxyphenyl)piperazin-1-yl)butyl-5-menthylpyrazolo[1,5-a]pyridin-2-ylcarbamide
(B52): N-4-(4-(2,3-dichlorophenyl)piperazin-1-yl)butyl-5-trifluoromethylpyrazolo[1,5-a]pyridin-2-ylcarbamide
(B53); N-4-(4-(2-methoxyphenyl)piperazin-1-yl)butyl-5-trifluoromethylpyrazolo[1,5-a]pyridin-2-ylcarbamide
(B 54): N-4-(4-(2,3-dichlorophenyl)piperazin-1-yl)butyl-6-bromopyrazolo[1,5-a]pyridin-2-ylcarbamide
(B13): N-4-(4-(2-methoxyphenyl)piperazin-1-yl)butyl-6-bromopyrazolo[1,5-a]pyridin-2-ylcarbamide
(B55): N-4-(4-(2,3-dichlorophenyl)piperazin-1-yl)butyl-6-chloropyrazolo[1,5-a]pyridin-2-ylcarbamide
(B56): N-4-(4-(2-methoxyphenyl)piperazin-1-yl)butyl-6-chloropyrazolo[1,5-a]pyridin-2-ylcarbamide
(B57); N-4-(4-(2,3-dichlorophenyl)piperazin-1-yl)butyl-6-fluoropyrazolo[1,5-a]pyridin-2-ylcarbamide
(B58): N-4-(4-(2-methoxyphenyl)piperazin-1-yl)butyl-6-fluoropyrazolo[1,5-a]pyridin-2-ylcarbamide
(B14): N-4-(4-(2-methoxyphenyl)piperazin-1-yl)butyl-3-methoxycarbonylpyrazolo[1,5-a]pyridin-2-ylcarbamide
(B15): N-4-(4-(2,3-dichlorophenyl)piperazin-1-yl)butyl-3-methoxycarbonylpyrazolo[1,5-a]pyridin-2-ylcarbamide
(B9): N-5-(4-(2,3-dichlorophenyl)piperazin-1-yl)pentylpyrazolo[1,5-a]pyridin-2-ylcarbamide
(B59): N-5-(4-(2,3-dichlorophenyl)piperazin-1-yl)pentyl-5-methoxypyrazolo[1,5-a]pyridin-2-ylcarbamide
(B60): N-5-(4-(2-methoxyphenyl)piperazin-1-yl)pentyl-5-methoxypyrazolo[1,5-a]pyridin-2-ylcarbamide
(B61): N-5-(4-(2,3-dichlorophenyl)piperazin-1-yl)pentyl-5-trifluoromethylpyrazolo[1,5-a]pyridin-2-ylcarbamide
(B62): N-5-(4-(2-methoxyphenyl)piperazin-1-yl)pentyl-5-trifluoromethylpyrazolo[1,5-a]pyridin-2-ylcarbamide
(B63): N-5-(4-(2,3-dichlorophenyl)piperazin-1-yl)pentyl-6-bromopyrazolo[1,5-a]pyridin-2-ylcarbamide
(B64): N-5-(4-(2-methoxyphenyl)piperazin-1-yl)pentyl-6-bromopyrazolo[1,5-a]pyridin-2-ylcarbamide
(B65): N-5-(4-(2,3-dichlorophenyl)piperazin-1-yl)pentyl-6-chloropyrazolo[1,5-a]pyridin-2-ylcarbamide
(B66): N-5-(4-(2-methoxyphenyl)piperazin-1-yl)pentyl-6-chloropyrazolo[1,5-a]pyridin-2-ylcarbamide
(B167): N-5-(4-(2,3-dichlorophenyl)piperazin-1-yl)pentyl-6-fluoropyrazolo[1,5-a]pyridin-2-ylcarbamide
(B68): N-5-(4-(2-methoxyphenyl)piperazin-1-yl)pentyl-6-fluoropyrazolo[1,5-a]pyridin-2-ylcarbamide
(B16): trans-N-(4-((4-(2-methoxyphenyl)piperazin-1-yl)methyl)cyclohex-1-yl)methyl-pyrazolo[1,5-a]pyridin-2-ylcarbamide
(B17): trans-N-(4-((4-(2,3-dichlorophenyl)piperazin-1-yl)methyl)cyclohex-1-yl)methyl-pyrazolo[1,5-a]pyridin-2-ylcarbamide
(B18): N-2-(4-(2,3-dichlorophenyl)piperazin-1-yl)ethylpyrazolo[1,5-a]pyridin-3-ylcarbamide
(B19): N-3-(4-(2,3-dichlorophenyl)piperazin-1-yl)propylpyrazolo[1,5-a]pyridin-3-ylcarbamide
(B20); N-4-(4-(2,3-dichlorophenyl)piperazin-1-yl)butylpyrazolo[1,5-a]pyridin-3-ylcarbamide
(B21): N-5-(4-(2,3-dichlorophenyl)piperazin-1-yl)pentylpyrazolo[1,5-a]pyridin-3-ylcarbamide
(B22): trans-N-(4-((4-(2-methoxyphenyl)piperazin-1-yl)methyl)cyclohex-1-yl)methyl-pyrazolo[1,5-a]pyridin-3-ylcarbamide
(B23): trans-N-(4-((4-(2,3-dichlorophenyl)piperazin-1-ylmethyl)cyclohex-1-yl)methyl-pyrazolo[1,5-a]pyridin-3-ylcarbamide
(B24): N-2-(4-(2,3-dichlorophenyl)piperazin-1-yl)ethylpyrazolo[1,5-a]pyridin-5-ylcarbamide
(B25): N-3-(4-(2,3-dichlorophenyl)piperazin-1-yl)propylpyrazolo[1,5-a]pyridin-5-ylcarbamide
(B26): N-4-(4-(2,3-dichlorophenyl)piperazin-1-yl)butylpyrazolo[1,5-a]pyridin-5-ylcarbamide
(B27): N-5-(4-(2,3-dichlorophenyl)piperazin-1-yl)pentylpyrazolo[1,5-a]pyridin-5-ylcarbamide
(B28); N-4-(4-(2-methoxyphenyl)piperazin-1-yl)butyl-3-bromopyrazolo[1,5-a]pyridin-5-ylcarbamide
(B29): N-4-(4-(2-methoxyphenyl)piperazin-1-yl)butyl-3-chloropyrazolo[1,5-a]pyridin-5-ylcarbamide
(B30): N-5-(4-(2-methoxyphenyl)piperazin-1-yl)pentylpyrazolo[1,5-a]pyridin-5-ylcarbamide
(B31): N-2-(4-(2,3-dichlorophenyl)piperazin-1-yl)ethylpyrazolo[1,5-a]pyridin-6-ylcarbamide
(B32): N-3-(4-(2,3-dichlorophenyl)piperazin-1-yl)propylpyrazolo[1,5-a]pyridin-6-ylcarbamide
(B33): N-4-(4-(2,3-dichlorophenyl)piperazin-1-yl)butylpyrazolo[1,5-a]pyridin-6-ylcarbamide
(B34): N-5-(4-(2,3-dichlorophenyl)piperazin-1-yl)pentylpyrazolo[1,5-a]pyridin-6-ylcarbamide
(B35): N-2-(4-(2,3-dichlorophenyl)piperazin-1-yl)ethyl-4,5,6,7-tetrahydropyrazolo[1,5-a]pyridin-2-ylcarbamide
(B36): N-3-(4-(2,3-dichlorophenyl)piperazin-1-yl)propyl-4,5,6,7-tetrahydropyrazolo[1,5-a]pyridin-2-ylcarbamide
(B37); N-4-(4-(2,3-dichlorophenyl)piperazin-1-yl)butyl-4,5,6,7-tetrahydropyrazolo[1,5-a]pyridin-2-ylcarbamide
(B39): N-4-(4-(2-methoxyphenyl)piperazin-1-yl)butyl-4,5,6,7-tetrahydropyrazolo[1,5-a]pyridin-2-ylcarbamide
(B40): N-4-(4-(2-methoxyphenyl)piperazin-1-yl)butyl-5-methyl-4,5,6,7-tetrahydropyrazolo[1,5-a]pyridin-2-ylcarbamide
(B89): N-5-(4-(2-methoxyphenyl)piperazin-1-yl)pentyl-4,5,6,7-tetrahydropyrazolo[1,5-a]pyridin-2-ylcarbamide
(B38): N-5-(4-(2,3-dichlorophenyl)piperazin-1-yl)pentyl-4,5,6,7-tetrahydropyrazolo[1,5-a]pyridin-2-ylcarbamide
(B41). N-2-(4-(2,3-dichlorophenyl)piperazin-1-yl)ethyl-4,5,6,7-tetrahydropyrazolo[1,5-a]pyridin-3-ylcarbamide
(B42): N-3-(4-(2,3-dichlorophenyl)piperazin-1-yl)propyl-4,5,6,7-tetrahydropyrazolo[1,5-a]pyridin-3-ylcarbamide
(B43): N-4-(4-(2,3-dichlorophenyl)piperazin-1-yl)butyl-4,5,6,7-tetrahydropyrazolo[1,5-a]pyridin-3-ylcarbamide
(B44): N-5-(4-(2,3-dichlorophenyl)piperazin-1-yl)pentyl-4,5,6,7-tetrahydropyrazolo[1,5-a]pyridin-3-ylcarbamide
(B90): N-4-(4-(2-methoxyphenyl)piperazin-1-yl)butyl-6-chloroimidazo[1,2-a]pyridin-2-ylcarbamide
(B91): N-4-(4-(2,3-dichlorophenyl)piperazin-1-yl)butyl-6-chloroimidazo[1,2-a]pyridin-2-ylcarbamide
(B92); N-4-(4-(2-methoxyphenyl)piperazin-1-yl)butyl-6-chloro-2-methylimidazo[1,2-a]pyridin-3-ylcarbamide
(B93): N-4-(4-(2-methoxyphenyl)piperazin-1-yl)butylimidazo[1,2-a]pyridin-6-ylcarbamide
(B 94): N-4-(4-(2,3-dichlorophenyl)piperazin-1-yl)butyl-1,2,4-triazolo[1,5-a]pyridin-2-ylcarbamide
(B95): N-4-(4-(2,3-dichlorophenyl)piperazin-1-yl)butylpyrazolo[1,5-b]pyridazin-2-ylcarbamide
(B96): N-4-(4-(2-methoxyphenyl)piperazin-1-yl)butyl-6-chloroimidazo[112-b]pyridazin-2-ylcarbamide
(B97): N-4-(4-(2-methoxyphenyl)piperazin-1-yl)butyl-6-chloro-2-phenylimidazo[1,2-b]pyridazin-3-ylcarbamide
   as well as pharmaceutically acceptable salts of these compounds.

Compounds of formulae (I), (II), (III), (IV), (V), (VI), (VII), (VIII) and (IX) as defined, are suitable as pharmaceutical preparations. The compounds according to the invention comprise affine or even highly affine ligands for D3 receptors.

The term “affine D3-ligand” covers compounds which in a radioligand experiment demonstrate bonding (see Hübner, H. et al. J. Med. Chem. 2000, 43, 756-762 and the section on “Biological Activity”) to human dopamine D3-receptors with a Ki-value of not more than 500 mM. For “affine” ligands of other receptors the definition applies by analogy.

The term “highly affine D3-ligands” covers compounds which in a radioligand experiment demonstrate bonding (see Hübner, H. et al. J. Med. Chem. 2000, 43, 756-762 and the section on “Biological Activity”) to human dopamine D3-receptors with a Ki-value of preferably not more than approximately 30 nM, particularly preferably not more than 3 nM. For “highly affine” ligands of other receptors the definition applies by analogy.

One aspect of the present invention concerns selective D3-ligands. The term “selective D3-ligands” covers compounds which in the radioligand experiment for the D3-receptor, as described in the following section “Biological Activity”, have a Ki value, which is lower by a factor of at least 10 than for at least five of the following seven receptors: dopamine receptors D1, D2long, D2short and D4.4, serotonin receptors 5-HT1A and 5-HT2 and alpha 1 adrenoceptor.

Another aspect of the invention concerns highly selective dopamine D3-ligands. The term “highly selective D3-ligands” covers compounds which in the radioligand experiment for the D3-receptor, as described in the following section “Biological Activity”, have a Ki-value, which is lower by a factor of at least 100 than for at least three, preferably all, of the dopamine receptors D1, D2long, D2short and D4.4.

D3-ligands can have an agonistic, antagonistic or partial agonistic effect on the D3-receptor. The corresponding intrinsic activities of the compounds according to the invention can be measured in mitogenesis assays, as described in the literature (Hübner, H. et al. J. Med. Chem. 2000, 43, 4563-4569 and Löber, S. Bioorg. Med. Chem. Lett. 2002, 12.17, 2377-2380). Depending on the pathophysiology of the underlying illness a stronger agnostic, a stronger antagonistic or a partial agonistic activity may be therapeutically desired.

Finally, some of the substances according to the invention also have significant affinity to other pharmacologically interesting receptors, such as for example the serotonin receptor, in particular the 5-HT1a-receptor, or the dopamine D2-receptor.

In place of a highly selective dopamine D3-receptor bond, depending on the type of illness to be treated, a bonding to a further receptor may be desired.

For example, for the treatment of schizophrenia a compound may be attractive which is a highly affine D3-ligand and at the same time an affine or even highly affine 5-HT1a-receptor ligand. In another embodiment of the invention for the treatment of dyskinesias a compound may be desired which apart from D3-modulatory characteristics also has D2-agonistic and 5-HT1a-modulatory characteristics. In other cases, e.g. in the treatment of urinal incontinence, a greater selectivity for the serotonin receptor may in fact be desirable.

The present invention therefore allows in an excellent manner fine tuning of the desired affinity, activity and selectivity in respect of various pharmacologically significant receptors, in particular the dopamine D3-receptors, but also for example in respect of the 5-HT1a-receptor or the D2-receptor.

A further object of the invention is therefore a pharmaceutical preparation containing one or more of the compounds of general formulae (I), (II), (III), (IV), (V), (VI), (VII), (VIII) and (IX) or one of the specifically listed compounds as defined above, possibly in the form of a pharmaceutically acceptable salt as well as a pharmaceutically acceptable adjuvant.

The invention also concerns the use of one or more of the compounds of general formulae (I), (II), (III), (IV), (V), (VI), (VII), (VIII) and (IX) or one of the specifically listed compounds, possibly in the form of a pharmaceutically acceptable salt, for the treatment of the indications mentioned here and the production of a pharmaceutical preparation for the indications mentioned here.

The term “treatment” of an illness covers in this patent application (a) therapy for a pre-existing illness and (b) prevention of an illness that has not yet or not yet fully developed, if there is a risk of such an illness occurring.

For the production of pharmaceutical preparations compounds according to the invention are preferably selected which are highly affine D3-ligands. Particularly preferable is the use of selective or even highly selective D3-ligands.

In another embodiment of the invention compounds are selected which are affine or even highly affine including or in particular for the 5-HT1a-receptor.

The compounds according to the invention have potential in the treatment or prevention of a series of illnesses, which in particular accompany dopamine metabolism or dopaminergic signalling cascade, or possibly serotoninergic signal transmission disorders.

An object of the invention is therefore the use of a compound according to the invention, as described in this patent application, including the claims and the examples, for the production of a pharmaceutical preparation for the treatment of illnesses which accompany dopamine metabolism and/or dopaminergic signalling cascade disorders.

Another object of the invention is the use of a compound according to the invention, as described in this patent application, including the claims and the examples, for the production of a pharmaceutical preparation for the treatment of illnesses which accompany serotonin metabolism and/or serotoninergic signal transmission disorders.

Illnesses in whose pathogenesis dopaminergic and or serotoninergic processes are involved, are in particular illnesses of the central nervous system. An object of the invention is therefore the use of a compound according to the invention, as described in this patent application, including the claims and examples, for the production of a pharmaceutical preparation for the treatment of central nervous system illnesses.

The term “central nervous system illnesses” in this patent application covers both disorders that have their origin in the central nervous system and whose symptoms are predominantly or exclusively noted in the central nervous system, such as psychoses, depressions or cognitive disorders, and illnesses which have their origin in the central nervous system, whose symptoms however at least in part can be noted in other target organs, such as extrapyramidal motor disturbances or hyperprolactinemias.

Examples of central nervous system illnesses which can be treated with the compounds according to the invention are:
(1) psychoses and anxiety disorders, including manias, idiopathic psychoses, schizophrenias, compulsive disorders, panic attacks, phobias, eating disorders, aggressive and autoagressive disorders, stereotypes and other personality disorders;
(2) drug dependency, e.g. cocaine, alcohol, opiate and nicotine addiction;
(3) emotional disorders, e.g. depressive disorders, in particular “major depression”, manic-depressive disorders, organically-induced depressions, e.g. in connection with neurodegenerative illnesses such as Parkinson's or Alzheimer's disease;
(4) motor disturbances, including tremors, rigor, dyskinesias, dystonias, such as in Parkinson's disease, parkinsonian syndrome (idiopathically, e.g. in Parkinson-plus-syndrome, or medication-induced, e.g. following L-dopa or neuroleptic treatment), Segawa syndrome, Tourette's syndrome, restless leg syndrome;
(5) sleeping disorders, including dopamine agonist triggered narcolepsy or sleeping disorders associated with Parkinson's disease;
(6) nausea: here dopamine antagonists can be used either alone or in combination with 5-HT3 antagonists;
(7) cognitive disorders and dementias;
(8) hyperprolactinemia; hyperprolactinomia and medically supported ablactation following pregnancy;
(9) glaucoma;
(10) attention deficit hyperactive syndrome (ADHS);
(11) autism, or disorders associated with autism, in particular in the case of compounds with strong serotonin active components;
(12) stroke, in particular in the case of compounds with strong serotoninergic active components.

A further therapeutic application that can be mentioned is the treatment and prevention of neurodegenerative diseases, since due to their neuroprotective effect the substances can delay or stop the destruction or loss of neurones as the cause or result of a pathophysiological episode. Such illnesses are for example amyotrophic lateral sclerosis, Alzheimer's disease, Huntington's chorea, epilepsy, Parkinson's disease or synucleopathias, e.g. of the Parkinson-plus-syndrome type.

Apart from the treatment of illnesses which clearly occur or continue with the involvement of the central nervous system, the substances according to the invention can also be used to treat other illnesses which are not clearly or not exclusively associated with the central nervous system. Such illnesses are in particular disorders of the urinary tract, such as sexual dysfunction, in particular male erectile dysfunction and urinary incontinence. For the treatment of urinary incontinence compounds with strong serotoninergic active components are particularly suitable.

An object of the invention is therefore the use of a compound according to the invention for the production of a pharmaceutical preparation for the treatment of disorders of the urinary tract, in particular of male erectile dysfunction and urinary incontinence.

Illnesses for which the compounds according to the invention are particularly suitable are schizophrenias, depressive disorders, L-dopa- or neuroleptic drug-induced motor disturbances, Parkinson's disease, Segawa syndrome, restless leg syndrome, hyperprolactinemia, hyperprolactinomia, attention deficit hyperactive syndrome (ADHS) and urinary incontinence.

Motor disturbances which are particularly open to therapy with the substances according to the invention are in particular
motor disturbances associated with Parkinson's disease, e.g. rigor, tremor, dystonias and dyskinesias,
Segawa syndrome
neuroleptic drug-induced (tardive) extrapyramidal motor disturbances, in particular dyskinesias, dystonias and akathisias,
L-dopa-induced extrapyramidal motor disturbances, in particular dyskinesias and dystonias,
restless leg syndrome.

Finally, the pharmaceutical preparations according to the invention, depending on the illness to be treated, can be in the form of a combined preparation for simultaneous or sequential administration.

For example, a sales unit, containing an L-dopa medication for treatment of Parkinson's disease, can also comprise a pharmaceutical composition containing one or more of the compounds according to the invention with, for example, a highly selective, partial agonist dopaminergic and/or serotoninergic profile of action. Here L-dopa and the compound according to the invention can be present in the same pharmaceutical formulation, e.g. a combined tablet, or also in different application units, e.g. in the form of two separate tablets. The two active substances can be administered simultaneously or separately as necessary.

In a combined preparation sequential administration can, for example, be achieved by the form of administration, e.g. an oral tablet, having two different layers with differing release profiles for the various pharmaceutically active components. It will be clear to the person skilled in the art that in the context of the present invention various forms of administration and application administration schemes are conceivable which all come within the subject-matter of the invention.

One embodiment of the invention therefore concerns a pharmaceutical preparation containing L-dopa or a neuroleptic drug and a compound according to the invention for simultaneous or timed sequential administration to the patient.

In another embodiment of the invention the sales unit can be a combined preparation or contain two application units, which contain two of the compounds according to the invention with different receptor profiles, e.g. a highly affine, highly selective D3-modulator and a highly affine 5-HT1a-modulator.

A further object of the invention is a method for treatment of an illness selected from among the illnesses listed in more detail above, through the administration of one or more of the compounds according to the invention, in each case either alone or in combination with other pharmaceutical preparations to a mammal, in need of such treatment, wherein the term “mammal” also and in particular includes humans.

Normally the pharmaceutical preparations according to the invention comprise a pharmaceutical composition which apart from the compounds according to the invention, as described above, contain at least one pharmaceutically acceptable carrier or adjuvant.

It will be clear to the person skilled in the art that the pharmaceutical formulation can be designed differently according to the envisaged administration route. Thus the pharmaceutical formulation can, for example, be adapted for intravenous, intramuscular, intracutaneous, subcutaneous, oral, buccal, sublingual, nasal, transdermal, inhalative, rectal or intraperitoneal administration.

Appropriate formulations and suitable pharmaceutical carriers or adjuvants, such as fillers, disintegrants, binding agents, lubricants, stabilisers, aromatics, antioxidants, preservatives, dispersion- or dissolution agents, buffers or electrolytes, will be known to the person skilled in the art in the area of pharmaceuticals and are for example described in the standard works such as Sucker, Fuchs and Speiser (“Pharmazeutische Technologie” (Pharmaceutical Engineering), Deutscher Apotheker Verlag, 1991) and Remington (“The Science and Practice of Pharmacy”, Lippincott, Williams & Wilkins, 2000).

In a preferred embodiment of the invention the pharmaceutical compositions containing the compounds according to the invention, are administered orally and can, for example, be in the form of capsules, tablets, powders, granulates, coated pills or a liquid.

Here the formulation can be designed as a rapid release form of administration, if a fast effect is desired. Appropriate oral formulations are, for example, described in EP 0 548 356 or EP 1 126 821.

If, on the other hand, a delayed release is desired, a formulation with delayed active substance release offers itself. Appropriate oral formulations are also known from the prior art. Alternative pharmaceutical preparations can, for example, be infusion or injection solutions, oils, suppositories, aerosols, sprays, plasters, microcapsules or microparticles.

The compounds of formulae (I) to (IX) were produced using methods that are in part already described in the literature (Bettinetti, L. et al. J. Med. Chem. 2002, 45, 4594-4597). In addition acid derivatives of type (A) were either obtained commercially or synthesised according to the instructions in the literature or the production methods for these were worked out in our laboratories and ten in the form of their carboxylic acid chlorides or alternatively through the use of special activation reagents such as hydroxybenzotriazole, hydroxyazabenzotriazole, HATU (Kienhöfer, A. Synlett 2001, 1811-1812) or TBTU (Knorr, R. Tetrahedron Lett. 1989, 30, 1927-1930) activated and with the free base of type (C) converted to the derivatives of formulae (I) to (IX):

A compound according to the invention according to formulae (I) to (IX) can be produced by the conversion of an acid derivative A 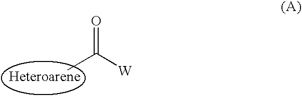
with a free base of general formula C 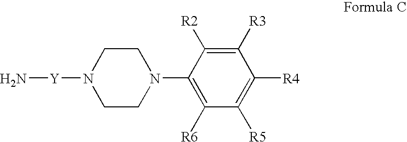
wherein:
W is selected from OH, Cl, Br or a group 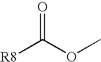
in which R8 stands for Alkyl;
heteroarene stands in each case for a group which is selected from 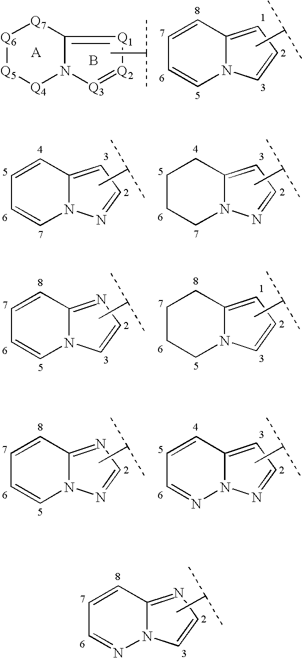
wherein
A, B, Q1, Q2, Q3, Q4, Q5, Q6 and Q7 in each case have the significance as defined in more detail above and wherein the crossed through bond for the heteroarenes stands for a bond of the —C(O)—W group to a C-atom of an aromatic ring of the heteroarene;
the heteroarene can be substituted once or a number of times, as defined above and in the claims;
Y, R2, R3, R4, R5 and R6 in each case have the significance as defined above and in the claims,
and wherein in the event that the substituent W is a hydroxyl group, the appropriate acid group prior to the conversion with the free base of general formula C is activated by addition of activation reagents such as hydroxybenzotriazole, hydroxyazabenzotriazole, HATU or TBTU.

W is preferably chlorine, bromine or OH particularly preferably chlorine or OH.

An important aspect in the synthesis of these target compounds is the efficient and cost-effective obtaining of preliminary synthesis stages. In the production of the pyrazolo[1,5-a]pyridine, an important structure class of this invention, the synthesis of the heterocyclic base body is performed via a cycloaddition reaction of n-aminopyridine with substituted propiolic acid esters (Bettinetti, L. et al. J. Med. Chem. 2002, 45, 4594-4597).

Previously the production of the pyridine preliminary stages has been achieved by n-amination with hydroxylamine derivatives as formulated below:

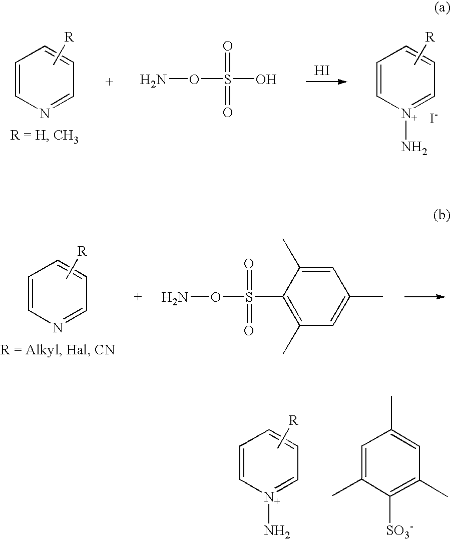

Here the application of reaction (a) is largely restricted to the conversion of pyridine or picoline, against which the amination of substituted pyridines according to reaction (b), because of the high costs of using the amination reagent hydroxylamine-O-mesitylsulfonic acid ester, is limited.

In this invention we describe an efficient and cost-effective method of synthesis for the production of variously substituted pyrazolo[1,5-a]pyridine on the basis of the synthesis of the necessary n-aminopyridine through conversion with O-(2,4-dinitrophenyl)hydroxylamine in accordance with (c) (Legault, C. et al. J. Org. Chem. 2003, 68, 7119-7122) and subsequent cycloaddition reaction with propiolic acid esters, as formulated by way of example in the following formula diagram (d): 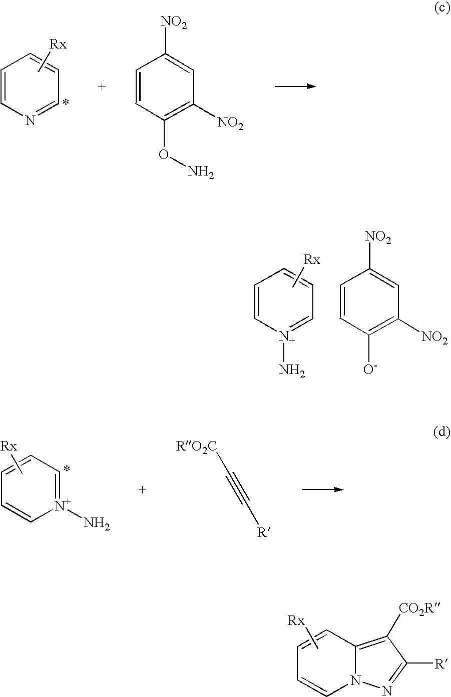
in which Rx stands for 0, 1, 2, 3 or 4 identical or different substituents selected from among halogen, alkyl, alkylcarbonyl, phenylcarbonyl, hydroxyalkyl, cyano, trifluoromethyl, and alkyloxycarbonyl, * identifies an unsubstituted CH group in which R′ is selected from hydrogen, alkyl, phenyl and alkyloxycarbonyl and in which R″ stands for alkyl.

An object of the invention is therefore the production of a carboxylic acid derivative of a pyrazolo[1,5-a]pyridine of general formula 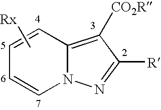
through the conversion of a pyridine of formula 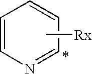
with O-(2,4-dinitrophenyl)hydroxylamine into an n-aminopyridine of formula 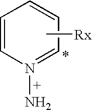
and subsequent cycloaddition reaction with a propiolic acid ester of formula 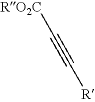
in which Rx stands for 0, 1, 2, 3 or 4 identical or different substituents selected from among halogen, alkyl, alkylcarbonyl, phenylcarbonyl, hydroxyalkyl, cyano, trifluoromethyl, and alkyloxycarbonyl, * identifies an unsubstituted CH group and in which R′ and R″ are selected from among hydrogen, alkyl, phenyl and alkyloxycarbonyl.
Synthesis of the Heteroarene Carboxylic Acid Derivates:
Production of Heteroarene Carboxylic Acids of Type A1:

### Indolizine-2-carboxylic acid

The production of the indolizine-2-carboxylic acid takes place by synthesis of the indolizine-2-carboxymethyl ester according to the literature (Bode, M. L. Chem. Soc. Perkin. Trans. 1993, 1809-1813) and subsequent hydrolysis.

For this 0.05 g (2.86 mmol) of the 2-indolizine carboxylic acid methyl ester are dissolved in 5 ml methanol and 5 ml THF. Then 2.5 ml 2n NaOH are added and agitation takes place for 10 hours at ambient temperature. The reaction solution is concentrated in the rotary evaporator and diluted with water, and then washed with hexane, adjusted with HCl to pH 3-4 and absorbed in diethyl ether. Following drying with MgSO₄ the solvent is evaporated.

Yield: 0.04 g (85%).

M.P.: 222° C. MS: m/z 161 (M⁺). IR (NaCl): 3429; 2924; 2852; 1741; 1664; 723. ¹H NMR (CDCl₃, 360 MHz) δ (ppm): 6.53-6.57 (m, 1H, H-6); 7.67-6.72 (m, 1H, H-7); 6.88 (s, 1H, H-1); 7.34 (d, J=9.0 Hz, 1H, H-8); 7.87-7.88 (m, 2H, H-3, H-5).

Production of Heteroarene Carboxylic Acids of Type A2:

### Pyrazolo[1,5-a]pyridine-2-carboxylic acid, pyrazolo[1,5-a]pyridine-3-carboxylic acid, pyrazolo[1,5-a]pyridine-5-carboxylic acid, pyrazolo[1,5-a]pyridine-6-carboxylic acid, 5-methoxypyrazolo[1,5-a]pyridine-2-carboxylic acid, 5-methylpyrazolo[1,5-a]pyridine-2-carboxylic acid, 5-trifluoromethyl-pyrazolo[1,5-a]pyridine-2-carboxylic acid, 6-bromopyrazolo[1,5-a]pyridine-2-carboxylic acid, 6-chloropyrazolo[1,5-a]pyridine-2-carboxylic acid, 6-fluoropyrazolo[1,5-a]pyridine-2-carboxylic acid,

The synthesis of these acid components takes place as described in the literature (Bettinetti, L. et al. J. Med. Chem. 2002, 45, 4594-4597).

The synthesis of 5-methoxypyrazolo[1,5-a]pyridine-2-carboxylic acid takes place analagously to the general conditions for synthesis of heteroarene carboxylic acids of type (A2).

Yield: 0.28 g (72%).

M.P.: 220° C. MS: m/z 192 (M⁺). IR (NaCl): 3050, 2939, 1704, 1652, 1411, 1230. ¹H NMR (DMSO, 360 MHz) δ (ppm): 3.84 (s, 314, CH₃O); 6.72 (dd, J=7.5 Hz, 2.5 Hz, 1H, H-6); 6.82 (s, 1H, H-3); 7.09 (d, J=2.5 Hz, 1H, H-4), 8.58 (d, J=7.5 Hz, 1H, H-7), 12.96 (s, 1H, COOH).

The synthesis of 5-methylpyrazolo[1,5-a]pyridine-2-carboxylic acid takes place analagously to the general conditions for synthesis of heteroarene carboxylic acids of type (A2).

Yield: 0.43 g (93%).

M.P.: 203° C. MS: m/z 176 (M⁺). IR (NaCl): 3133, 3050, 1697, 1405, 1270, 937. ¹H NMR (DMSO, 360 MHz) δ (ppm): 6.87-6.90 (m, 1H, H-6); 6.91 (s, 1H, H-3); 7.52 (s, 1H, H-4); 8.62 (d, J=7.4 Hz, 1H, H-7); 13.00 (br s, 1H, COOH). ¹³C NMR (DMSO, 90 MHz) δ (ppm): 163.6, 145.3, 140.5, 134.6, 128.2, 117.1, 116.9, 98.5, 20.6.

The synthesis of 5-trifluoromethylpyrazolo[1,5-a]pyridine-2-carboxylic acid takes place analagously to the general conditions for synthesis of heteroarene carboxylic acids of type (A2).

Yield: 0.54 g (84%).

M.P.: 230° C. MS: m/z 230 (M⁺). IR (NaCl): 3445, 1698, 1495, 1460, 1331, 1241. ¹H NMR (DMSO, 360 MHz) δ (ppm): 7.30 (dd, J=7.4, 2.1 Hz, 1H, H-6); 7.32 (s, 1H, H-3); 8.36 (s, 1H, H-4); 8.97 (dd, J=7.4 Hz, 0.7 Hz, 1H, H-7); 13.34 (br s, 1H, COOH). ¹³C NMR (DMSO, 90 MHz) δ (ppm): 163.0 (CO₂H), 146.6 (C-7), 138.8 (C-2), 130.6 (C-3a), 124.4 (q, J=34 Hz, C-5), 123.3 (q, J=273 Hz, CF₃), 118.0 (q, J=5 Hz, C-6), 109.6 (q, J=3 Hz, C-4), 103.0 (C-3).

The synthesis of 6-bromopyrazolo[1,5-a]pyridine-2-carboxylic acid takes place analagously to the general conditions for synthesis of heteroarene carboxylic acids of type (A2).

Yield: 0.27 g (70%).

M.P.: 226° C. MS: m/z 240 (M⁺), 242 ((M+2)⁺). IR (NaCl): 3135, 3070, 1701, 1402, 1265, 920. ¹H NMR (DMSO, 360 MHz) δ (ppm): 7.12 (d, J=0.72 Hz, 1H, H-3); 7.44 (dd, J=9.6 Hz, J=1.8 Hz, 1H, H-5); 7.78 (dd, J=9.6 Hz, J=0.72 Hz, in, H-4); 9.16 (s, 1H, H-7); 13.19 (br s, in, COOH). ¹³C NMR (DMSO, 90 MHz) δ (ppm): 163.2, 145.5, 139.1, 129.2, 127.5, 120.2, 108.4, 100.8.

The synthesis of 6-chloropyrazolo[1,5-a]pyridine-2-carboxylic acid takes place analagously to the general conditions for synthesis of heteroarene carboxylic acids of type (A2).

Yield: 0.35 g (80%).

M.P.: 233° C. MS: m/z 196 (M⁺), 198 ((M+2)⁺). IR (NaCl): 3444, 3080, 1699, 1506, 1495, 1269, 1063. ¹ NMR (DMSO, 360 MHz) δ (ppm): 7.13 (d, J=0.9 Hz, 1H, H-3); 7.36 (dd, J=39.5 Hz, 1.8 Hz, 1H, H-5); 7.83 (dd, J=9.5 Hz, 0.9 Hz, 1H, H-4); 8.97 (br s, 1H, H-7). ¹³C NMR (DMSO, 90 MHz) δ (ppm): 163.1, 145.7, 139.0, 127.1, 125.7, 121.3, 120.0, 100.7.

The synthesis of 6-fluoropyrazolo[1,5-a]pyridine-2-carboxylic acid takes place analagously to the general conditions for synthesis of heteroarene carboxylic acids of type (A2).

Yield: 0.17 g (71%).

M.P.: 245° C. MS: m/z 180 (M⁺). IR (NaCl): 3135, 3080, 1698, 1510, 1494, 1269, 1064. ¹H NMR (DMSO, 360 MHz) δ (ppm): 7.14 (d, J=0.9 Hz, 1H, H-3); 7.43 (ddd, J=9.8 Hz, 8.4 Hz, 2.3 Hz, 1H, H-6); 7.88 (ddd, J=9.8 Hz, 5.9 Hz, 0.7 Hz, 1H, H-4); 9.05 (br d, J=4.8 Hz, 1H, H-7). ¹³C NMR (DMSO, 90 MHz) δ ppm): 163.1 (C-2H), 153.9 (d, J=236 Hz, C-6), 145.6 (d, J=3 Hz, C-3a), 138.3 (C-2), 120.0 (d, J=9 Hz, C-4), 116.8 (d, J=26 Hz, C-7), 116.3 (d, J=41 Hz, C-5), 100.7 (C-3).

Production of Heteroarene Carboxylic Acids of Type A3:

### 3-bromopyrazolo[1,5-a]pyridine-2-carboxylic acid, 3-chloropyrazolo[1,5-a]pyridine-2-carboxylic acid, 3-bromopyrazolo[1,5-a]pyridin-5-carboxylic acid, 3-chloropyrazolo[1,5-a]pyridin-5-carboxylic acid

0.10 g (0.6 mmol) pyrazolo[1,5-a]pyridine-2-carboxylic acid (A2) and 0.13 g (0.75 mmol) N-bromosuccinimide are mixed with 7 ml chloroform under a protective gas atmosphere and agitated for 55 hours at ambient temperature. Then the solvent is evaporated in the vacuum; cleaning with flash chromatography (CH₂Cl₂-MeOH: 90-10) produces 3-bromopyrazolo[1,5-a]pyridine-2-carboxylic acid.

Yield: 0.11 g (73%).

M.P.; >300° C. dec. MS: m/z 240 (M⁺), 242 ((M+2)⁺). IR (NaCl): 3382, 1643, 1577, 1523, 1467, 1396. ¹H NMR (DMSO, 360 MHz) δ ppm); 7.06-7.10 (m, 1H, H-6); 7.34-7.41 (m, 1H, H-5); 7.59 (d, J=8, 9 Hz, 1H, H-4); 9.24 (d, J=36.7 Hz, 1H, H-7).

The synthesis of 3-chloropyrazolo[1,5-a]pyridine-2-carboxylic acid takes place analagously to the general conditions for synthesis of heteroarene carboxylic acids of type (A3).

Yield: 60 mg (49%).

M.P.: >300° C. dec. MS: m/z 196 (M⁺), 198 ((M+2)⁺). IR (NaCl): 3396, 3099, 1633, 1604, 1504, 1403, 1348. ¹H NMR (DMSO, 360 MHz) δ (ppm): 7.04-7.08 (m, 1H, H-5); 7.34-7.38 (m, 1H, H-6); 7.61 (d, J=9.2 Hz, 1H, H-4); 9.08 (br d, J=5.7 Hz, 1H, H-7).

The synthesis of 3-bromopyrazolo[1,5-a]pyridine-5-carboxylic acid takes place analagously to the general conditions for synthesis of heteroarene carboxylic acids of type (A3).

Yield: 0.13 g (87%).

M.P.: >300° C. dec. MS: m/z 240 (M⁺), 242 ((M+2)⁺). IR (NaCl): 3382, 1643, 1577, 1523, 1467, 1396. ¹H NMR (DMSO, 360 MHz) δ (ppm): 7.38 (d, J=6.7 Hz, 1H, H-6); 8.07 (s, 1H, H-4); 8.25 (s, 1H, H-2); 8.75 (d, J=7.1 Hz, 1H, H-7).

The synthesis of 3-chloropyrazolo[1,5-a]pyridine-5-carboxylic acid takes place analagously to the general conditions for synthesis of heteroarene carboxylic acids of type (A3).

Yield: 75 mg (99%).

M.P.: 180° C. MS: m/z 196 (M⁺), 198 ((M+2)⁺). IR (NaCl): 3406, 3100, 1710, 1576, 1529, 1509, 1396. ¹H NMR (DMSO, 360 MHz) δ ppm): 7.42-7.45 (m, 1H, H-6); 8.10 (s, 1H, H-4); 8.16 (s, 1H Hz, H-2); 8.63 (br d, J=7.1 Hz, In, H-7).

Production of Heteroarene Carboxylic Acids of Type A4:

### 4,5,6,7-tetrahydropyrazolo[1,5-a]pyridine-2-carboxylic acid, 4,5,6,7-tetrahydropyrazolo[1,5-a]pyridin-3-carboxylic acid, 4,5,6,7-tetrahydro-5-methylpyrazolo[1,5-a]pyridine-2-carboxylic acid

0.20 g (1.2 mmol) pyrazolo[1,5-a]pyridine-2-carboxylic acid (A2) are dissolved in 10 ml ethanol and hydrated with 40 mg Pd/C 10% at 16 bar 12 pressure and 80° C. in a 100 ml pressure tube for 4 hours. Filtering off of Pd-charcoal and reddening produces 4,5,6,7-tetrahydropyrazolo[1,5-a]pyridine-2-carboxylic acid.

Yield: 0.20 g (98%).

M.P.: 118° C. MS: m/z 166 (M⁺), IR (NaCl): 3135, 2951, 2867, 1717, 1215, 771. ¹H NMR (DMSO, 360 MHz) δ (ppm): 1.74-1.81 (m, 2H, H-5); 1.94-2.00 (m, 2H, H-6); 2.75 (t, J=6.4 Hz, 2H, H-4); 4.08-4.11 (m, 2H, H-7), 6.41 (s, 1H, H-3). ¹³C NMR (DMSO, 90 MHz) δ (ppm): 163.4, 142.2, 140.2, 105.4, 48.0, 22.7, 22.0, 19.6.

The synthesis of 4,5,6,7-tetrahydropyrazolo[1,5-a]pyridine-3-carboxylic acid takes place analagously to the general conditions for synthesis of heteroarene carboxylic acids of type (A4).

Yield: 0.30 g (37%).

M.P.: 210° C. MS: m/z 166 (M⁺), IR (NaCl): 3399, 2957, 2921, 1705, 1551, 1230. ¹H NMR (DMSO, 360 MHz) δ (ppm): 1.75-1.82 (m, 2H, H-5); 1.91-1.98 (m, 2H, H-6); 2.93 (t, J=6.4 Hz, 2H, 1H-4); 4.17-4.20 (t, J=6.0 Hz, 2H, H-7), 7.72 (s, 1H, H-2).

The synthesis of racemic 4,5,6,7-tetrahydro-5-methylpyrazolo[1,5-a]pyridine-3-carboxylic acid takes place analagously to the general conditions for synthesis of heteroarene carboxylic acids of type (A4).

Yield: 0.197 g (96%).

M.P.: 163° C. MS: m/z 180 (M⁺), IR (NaCl): 3343, 2960, 2927, 2871, 1691, 1396, 1240, 780. ¹H NMR (DMSO, 360 MHz) δ (ppm): 1.14 (d, J=6.7 Hz, 3H, CH₃); 1.70-1.82 (m, 1H, H-5); 1.97-2.11 (m, 2H, H-6); 2.39 (dd, J=16.3 Hz, J=10.3 Hz, 1H, H-4); 2.96 (dd, J=16.3 Hz, J=4.6 Hz, 1H, H-4); 4.09-4.17 (m, 1H, H-7); 4.37-4.43 (m, 1H, H-7); 6.56 (d, 1H, 72 Hz, H-3).

Production of Heteroarene Carboxylic Acids of Type A5:

### Indolizine-1-carboxylic acid

The production of the indolizine-1-carboxylic acid takes place by synthesis of the indolizine-1-carboxylic acid methyl ester in accordance with the literature (Zhang, L. Feng, L., Sun, L. Hu, Y., Hu, H., Synthesis 2000, 1733-1737) and subsequent hydrolysis.

For this 0.2 g (1.14 mmol) of the indolizine carboxylic acid methyl ester are dissolved in 5 ml methanol and 5 ml THF. Then 2.5 ml 2n NaOH are added and agitation takes place for 10 hours at ambient temperature. The reaction solution is concentrated in the rotary evaporator and diluted with water, then washed with hexane, adjusted with HCl to pH 3-4 and absorbed in diethyl ether. Following drying with MgSO₄ the solvent is evaporated.

Yield: 0.072 g (39%).

M.P.: 196-198° C. MS: m/z 161 (M⁺). IR NaCl): 3362; 2925; 2853; 1633; 720.

Production of Heteroarene Carboxylic Acids of Type A6:

### Tetrahydroindolizine-2-carboxylic acid

0.06 g (0.375 mmol) indolizine-2-carboxylic acid (A1) are dissolved in 10 ml ethanol and hydrated with 13 mg Pd/C 10% at 16 bar H₂-pressure and at 80° C. in a 100 ml pressure tube for 6 hours. Filtering off of Pd-charcoal and evaporation of the solvent produce 5,6,7,8-tetrahydroindolizine-2-carboxylic acid.

Yield: 0.20 g (98%).

M.P.: 131-134° C. MS: m/z 166 ((M+H)⁺), IR (NaCl): 3135, 2951, 2867, 1717, 1215, 771. ¹H NMR (DMSO, 360 MHz) δ (ppm); 1.69-1.76 (m, 2H, H-6); 1.82-1.88 (m, 2H, H-7); 2.65-2.69 (m, 2H, H-5); 3.90-3.95 (m, 2H, H-8), 6.05 (s, 1H, H-3); 7.17 (s, 1H, H-1).

Production of Heteroarene Carboxylic Acids of Type A7:

### 1-cyano-2-methylindolizine-3-carboxylic acid

The 1-cyano-2-methylindolizine-3-carboxylic acid ethyl ester (0.05 g (0.21 mmol)) purchased from Ambinter, Paris (F) is dissolved in 5 ml methanol and 5 ml THF. Then 2.5 ml 2n NaOH are added and agitation takes place for 4 hours at ambient temperature. The reaction solution is concentrated in the rotary evaporator and diluted with water, then washed with hexane, adjusted with HCl to pH 3-4 and absorbed in diethyl ether. Following drying with MgSO₄ the solvent is evaporated.

Yield: 0.04 g (90%).

MS: m/z 201 ((M+H)⁺).

Production of Heteroarene Carboxylic Acids of Type A8:

### Imidazo[1,2-a]pyridine-6-carboxylic acid

For the synthesis 0.1 g (0.57 mmol) imidazo[1,2-a]pyridine-6-carboxylic acid methyl ester (Bionet Research Ltd., Camelford (UK)) are dissolved in 5 ml methanol and 5 ml THF. Then 5 ml 2n NaOH are added and agitation takes place for 4 hours at ambient temperature. The reaction solution is concentrated in the rotary evaporator and diluted with water, then washed with hexane, adjusted with HCl to pH 3-4 and absorbed in diethyl ether. The aqueous phase is lyophilised by freeze drying, and then the residue is washed out with ethanol and filtered. Following drying with MgSO₄ the solvent is evaporated.

Yield: 0.02 g (22%) white resinous substance.

MS: m/z 163 ((M+H)⁺). IR (NaCl): 3378; 1643.

Production of Heteroarene Carboxylic Acids of Type A9:

### 1,2,4-triazolo[1,5-a]pyridine-2-carboxylic acid

The production of the 1,2,4-triazolo[1,5-a]pyridine-2-carboxylic acid takes place by synthesis of the 1,2,4-triazolo[1,5-a]pyridine-2-carboxylic acid ethyl ester in accordance with the literature (Gomez, E., Avedano, C., McKillop, A., Tetrahedron 1986, 2625-2634) and subsequent hydrolysis.

For this 0.05 g (0.26 mmol) of the 1,2,4-triazolo[1,5-a]pyridine-2-carboxylic acid ethyl-ester are dissolved in 5 ml methanol. Then 2.5 ml 5n NaOH are added and agitation takes place for 4 hours at ambient temperature. The reaction solution is concentrated in the rotary evaporator and diluted with water, then washed with hexane, adjusted with HCl to pH 3-4 and absorbed in diethyl ether and ethyl acetate. Following drying with MgSO₄ the solvent is evaporated.

Yield: 0.010 g (23%) white resin.

MS: m/z 164 ((M+H)⁺).

Production of Heteroarene Carboxylic Acids of Type A10:

### Pyrazolo[1,5-b]pyridazine-2-carboxylic acid

The production of the pyrazolo[1,5-b]pyridazine-2-carboxylic acid takes place by synthesis of the dimethylpyrazolo[1,5-b]pyridazine-2,3-dicarboxylate in accordance with the literature (Kobayashi, Y. Kutsuma, T., Morinaga, K., Chem. Pharm. Bull. 1971, 2106-2115) and subsequent acid hydrolysis and decarboxylation.

For this 0.20 g (1.0 mmol) of the dimethylpyrazolo[1,5-b]pyridazine-2,3-dicarboxylate are suspended in 10 ml H₂SO₄ (40%) and then heated for 2.5 hours to 110° C. The reaction solution is cooled to ambient temperature and then to 0° C. The cooled solution is neutralised with NaOH (5N) and adjusted with HCl to pH 3. Then it is absorbed in diethyl ether. Following drying with MgSO₄ the solvent is evaporated.

Yield: 0.045 g (28%) white solid matter.

M.P.: 263-265° C.). MS: m/z 164 ((M+H)⁺).

Efficient Synthesis of Substituted Pyrazolo[1,5-a]Pyridines:

The efficient synthesis of substituted pyrazolo[1,5-a]pyridines developed by us is described via the example of production of pyrazolo[1,5-a]pyridin-5-yl-carboxylic acid.

Production of n-aminopyridine:

### N-amino-4-hydroxymethylpyridinium 2,4-dinitrophenolate

0.75 g (6.89 mmol) 4-hydroxymethylpyridine are droppered into a solution of 1.50 g (7.54 mmol) O-(2,4-dinitrophenyl)hydroxylamine in 10 ml methylene chloride under a protective gas atmosphere and agitated for 21 hours at ambient temperature. Following the addition of diethyl ether the precipitated solid matter is filtered off and washed with ether. The product is used without purification for the next reaction.

Yield: 1.78 g (84%).

M.P.: 108° C.

Production of Pyrazolo[1,5-a]Pyridine Carboxylic Acids:

### 5-hydroxymethylpyrazolo[1,5-a]pyridin-3-ylcarboxylic acid methyl ester

0.97 g (7 mmol) calcium carbonate are added to a solution of 1.5 g (4.9 mmol) N-amino-4-hydroxymethylpyridinium 2,4-dinitrophenolate in 8 ml dry DMF and 0.45 g (5.3 mmol) propiolic acid methylester droppered in. After 20 hours of agitation at ambient temperature filtering off is performed, the solvent evaporated, the residue absorbed in water and extracted with diethyl ether and the organic phase dried with MgSO₄. Evaporation of the solvent and purification with flash chromatography on silica gel (EtOAc benzine: 3-7) produces 5-hydroxymethylpyrazolo[1,5-a]pyridin-3-ylcarboxylic acid methyl ester.

Yield: 0.46 g (46%).

The analytical data and the further synthesis steps for obtaining pyrazolo[1,5-a]pyridin-5-ylcarboxylic acid are described in the literature (Bettinetti, L. et al. J. Med. Chem. 2002, 45, 4594-4597).

Synthesis of Amine Components:

Production of Type C1 Amines:

### 4-(4-(2-(3-dichlorophenyl)piperazin-1-yl)alkylamine, 4-(4-(2-methoxyphenyl)piperazin-1-yl)alkylamine

For the production of the type (C1) arylpiperazinylamine commercially available 2-methoxy- or 2,3-dichlorophenylpiperazine, for example, can be alkylated with bromobutylphthalimide in xylol. Subsequent hydrazinolysis of the phthalimide substituted structures provides the type (A1) primary amine. This is explained by way of example in the following reaction diagram: 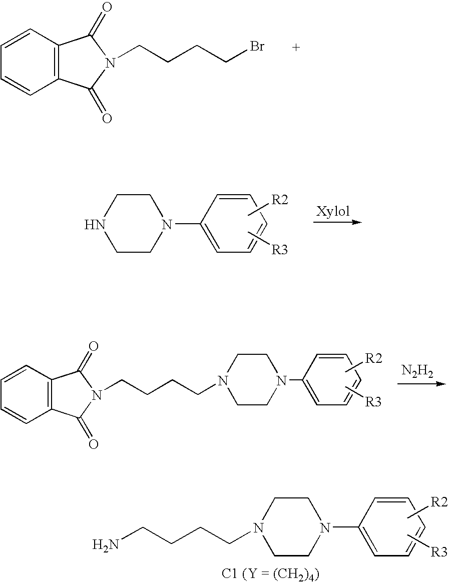
2.3 g (10 mmol) 2,3-dichlorophenylpiperazine (base) are dissolved in 10 ml xylol and heated to 70° C. Then 1.4 g (5 mmol) 4-bromobutylphthalimide (dissolved in 20 ml xylol) are droppered in and the reaction mixture is heated for 24 hours at 125° C. Following cooling of the mixture to 0° C. filtering off is performed and the filtrate evaporated. The resultant N-4-(4-(2,3-dichlorophenyl)piperazin-1-yl)butylphthalimide is purified by flash chromatography on SiO₂ with ethyl acetate.

Yield: 4.0 g (92%).

A solution of 0.45 ml 80% hydrazine hydrate (2.5 eq) in 5 ml ethanol is droppered into a suspension of N-4-(4-(2,3-dichlorophenyl)piperazin-1-yl)butylphthalimide in 40 ml ethanol. The mixture is heated for 3 hours with recycling and then cooled to ambient temperature, the resultant solid matter is filtered off and the ethanolic solution is evaporated in the vacuum. Purification with flash chromatography (CH₂Cl₂-MeOH-Me₂EtN:90-8-2) produces the free base 4-(4-(2,3-dichlorophenyl)piperazin-1-yl)butylamine.

Yield: 0.900 g (60%).

MS: m/z 301 (M⁺), 303 ((M+4)⁺), 305 (M+4)⁺); IR: (NaCl): 3397, 2939, 2817, 1641, 1572, 1500, 1482, 1376, 1240, 1152, 1118, 1023, 917, 791, 749, 698, 661. ¹H NMR (CDCl₃, 360 MHz) δ (ppm): 1.48-1.64 (m, 4H, CH₂—CH₂); 2.44 (t, J=7.6 Hz, 2H, CH₂N); 2.64 (m, 4H, pip); 2.72-2.76 (m, 2H, H₂N—CH₂); 3.07 (m, 4H, pip); 6.93-6.99 (m, 1H, phenyl H-5); 7.11-7.17 (m, 2H, phenyl H-4, phenyl H-6).

Production of Type C2 Amines:

### 4-(4-(3-chloro-2-methoxyphenyl)piperazin-1-yl)butylamine, 4-(4-(2,3-difluorophenyl)piperazin-1-yl)butylamine

An alternative method of synthesis for obtaining variously substituted type (C2) phenylpiperazinylalkylamines is the reaction of the piperazine with a cyanoalkylhalogenide of appropriate chain length, as explained by way of example in the following reaction diagram:

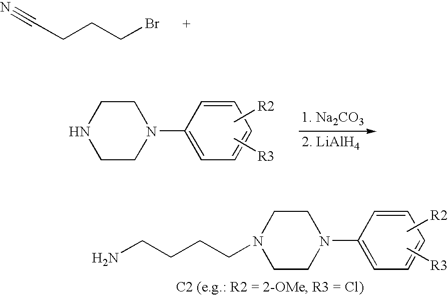

The corresponding 2,3-disubstituted phenylpiperazines are accessible through palladium-catalysed amination of 2,3-substituted halogen aromatic compounds with piperazine:

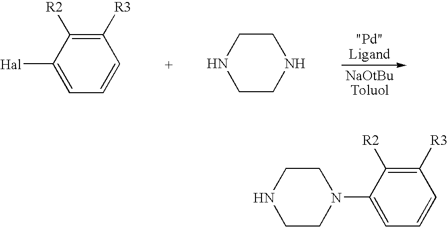

Thus for the synthesis of 4-(4-(3-chloro-2-methoxyphenyl)piperazin-1-yl)butylamine 1.35 g NaOtBu (14 mmol), 0.024 g Pd(II)acetate (0.5 mol %) and 0.12 g P(OtBu)₃ (2 mol %) are added to 1.7 g (10 mmol) piperazine (base) and dissolved with 1.3 ml dichloroanisol (10 mmol) in 20 ml toluene. After 21 hours of heating to 70° C. the mixture is cooled to ambient temperature, filtered and the filtrate then evaporated in order to obtain 4-(3-chloro-2-methoxyphenyl)piperazine.

Yield: 0.8 g (37%).

0.8 g (3.7 mmol) 4-(3-chloro-2-methoxyphenyl)piperazine and 0.8 g (7.5 mmol) Na₂CO₃ are dissolved in 20 ml acetonitrile, heated for 15 hours with recycling, then cooled to ambient temperature and the solution evaporated in the vacuum. The residue is absorbed in water and the aqueous phase extracted with methylene chloride, this is dried (with MgSO₄) and the solvent is evaporated. Purification with flash chromatography (CHCl₃-EtOAc:1-1) produces 4-(4-(3-chloro-2-methoxyphenyl)piperazin-1yl)butyronitrile.

Yield; 0.4 g (35%).

Then 0.15 g 4-(4-(3-chloro-2-methoxyphenyl)piperazin-1yl)butyronitrile (0.5 mmol) are dissolved in 5 ml dry diethyl ether and cooled to 0° C. Then 1.0 ml LiAli₄ solution (1 M in diethyl ether) is slowly droppered in and agitation takes place for 1 hour at ambient temperature. Following cooling again to 0° C. saturated NaHCO₃ solution is added, filtration is performed through a fritted glass filter with Celite/MgSO₄/Celite and washing is performed with methylene chloride. Evaporation of the filtrate produces 4-(4-(3-chloro-2-methoxyphenyl)piperazin-1-yl)butylamine.

Yield: 0.143 g (96%).

MS: m/z 297 (M⁺), 299 ((M+2)⁺), 301 ((M+4)⁺). IR: (NaCl): 3386, 2937, 2821, 1635, 1584, 1540, 1474, 1450, 1251, 1132, 1001, 964, 782, 744, 680, 668. ¹H NMR (CDCl₃, 360 MHz) δ (ppm): 1.60-1.67 (m, 4H, CH₂—CH₂); 2.41-2.45 (m, 2H, H₂N—CH₂); 2.61 (m, 4H, pip); 3.14 (m, 4H, pip); 3.22-3.26 (m, 2H, CH₂N); 3.86 (s, 1H, OCH₃); 6.79-6.82 (m, 1H, phenyl); 6.95 (dd, J=8.0 Hz, J=8.0 Hz, 1H, phenyl H-5); 7.00 (dd, J=1.8 Hz, J=8.0 Hz, 1H, phenyl).

For the production of 4-(4-(2,3-difluorophenyl)piperazin-1-yl)butylamine 0.56 g (5 mmol) piperazine (base) are dissolved with 0.675 g NaOtBu (7 mmol), 0.046 g Pd₂(dba)₃ (0.5 mol %), 0.093 g BINAP (2 mol %) and 0.56 ml (5 mmol) 1-bromine-2,3-difluorobenzol in 20 ml toluene and heated for 18 hours to 151° C. Following cooling of the reaction solution to ambient temperature filtering off is performed and the filtrate is evaporated to obtain 2,3-difluorophenylpiperazine.

Yield: 0.55 g (55%).

The subsequent conversion to 4-(4-(2,3-difluorophenyl)piperazin-1-yl)butylamine takes place analagously to the synthesis described above of type (B2) amines.

Yield: 0.173 g (78% over 2 reaction steps).

MS: m/z 269 (M⁺). IR: NaCl): 3355, 2939, 2823, 1621, 1585, 1504, 1478, 1269, 1247, 1143, 1007, 774, 714, ¹H NMR (CDCl₃, 360 MHz) δ (ppm): 1.47-1.60 (m, 4H, CH₂—CH₂); 2.39-2.44 (m, 2H, H₂N—CH₂); 2.61-2.65 (m, 4H, pip); 2.71-2.75 (m, 2H, CH₂N); 3.12-3.15 (m, 4H, pip); 6.67-6.71 (m, 1H, phenyl); 6.73-6.80 (m, 1H, phenyl); 6.92-6.99 (m, 1H, phenyl).

Production of Type C3 amines:

### 4-(4-(2,3-dihydrobenzofuran-7-yl)piperazin-1-yl)butylamine, 4-(4-(chroman-8-yl)piperazin-1-yl)butylamine

The synthesis takes place to begin with analagously to the literature (Kerrigan, F. Tetrahedron Lett. 1998, 2219-2222) until 2,3-dihydrobenzofuran-7-ylpiperazine has been obtained with a yield of 54% over 4 reaction steps. Then the free base is alkylated analagously to the general conditions for the synthesis of type (C2) amines and the resultant nitrile is reduced to 4-(4-(2,3-dihydrobenzofuran-7-yl)piperazin-1-yl)butylamine.

Yield: 0.27 g (86% over 2 reaction steps).

MS: m/z 275 (M⁺). IR: (NaCl): 3359, 2939, 2820, 1609, 1487, 1456, 1254, 1190, 1132, 1012, 942, 870, 755, 661. ¹H NMR (CDCl₃, 360 MHz) δ (ppm): 1.43-1.63 (m, 4H, CH₂—CH₂); 2.34-2.40 (m, 2H, H₂N—CH₂); 2.62 (m, 4H, pip); 2.72-2.74 (m, 2H, O—CH₂—CH₂); 3.15-3.21 (m, 6H, pip, CH₂N); 4.56-4.61 (m, 2H, O—CH₂—CH₂); 6.69-6.71 (m, 1H, phenyl); 6.77-6.86 (m, 2H, phenyl).

The production of 4-(4-(chroman-8-ylpiperazin-1-yl)butylamine takes place analagously to the general conditions for synthesis of type (C3) amines.

Yield: 0.058 g (57% over 2 reaction steps).

MS: m/z 289 (M⁺). IR: NaCl): 3354, 2933, 2870, 2814, 1664, 1479, 1461, 1247, 1196, 1024, 870, 737, ¹H NMR (CDCl₃, 360 MHz) δ (ppm): 1.46-1.59 (m, 4H, CH₂—CH₂); 1.96-2.03 (m, 2H, O—CH₂—CH₂—CH₂); 2.39-2.44 (m, 2H, CH₂—N); 2.65 (m, 4H, pip); 2.70-2.74 (m, 2H, O—CH₂—CH₂—CH₂); 2.77-2.80 (m, 2H, CH₂—NH₂); 3.08 (m, 4H, pip) 4.24-4.27 (m, 2H, O—CH₂—CH₂—CH₂); 6.71-6.79 (m, 3H, phenyl).

Production of Type C4 Amines:

### Trans-4-(4-aminomethylcyclohex-1-ylmethyl)-1-(2-methoxyphenyl)piperazine, trans-4-(4-aminomethylcyclohex-1-ylmethyl)-1-(2,3-dichlorophenyl)piperazine

The synthesis of the amine components with methylcyclohexylmethyl-spacers between amine nitrogen and piperazine is performed as follows;

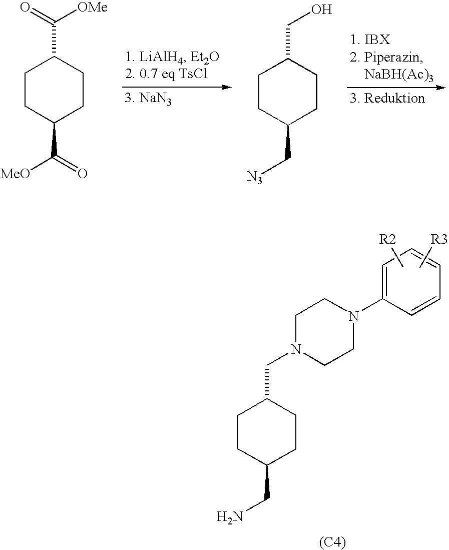

Starting with 1,4-cyclohexylidene dicarboxylic acid dimethyl ester the conversion to 4-azidomethylcyclohex-1-ylmethanol takes place in accordance with the literature (Watanabe, T. Chem. Pharm. Bull. 1995, 43, 529-531). Then oxidation to the aldehyde, reductive amination with the corresponding phenylpiperazines and reduction of the azido group to the primary amine provide the type (C4) amines.

For the synthesis of trans-4-azidomethylcyclohex-1-ylcarbaldehyde 0.10 g (0.6 mmol) trans-4-azidomethylcyclohex-1-ylmethanol are dissolved in 4 ml dry DMSO and following addition of 0.21 g (0.77 mmol) IBX (1-hydroxy-1,2-benziodoxol-3(114)-one-1-oxide) agitated for 5 hours at ambient temperature. Then diethyl ether and NaHCO₃ solution are added and the organic phase is separated off. This is again washed with NaUCO₃ solution and water and dried over MgSO₄. The solvent is evaporated in the vacuum.

Yield: 75 mg (76%).

MS: m/z 167 (M⁺); IR: (NaCl): 2927, 2856, 2097, 1723, 1452. ¹H NMR (CDCl₃, 360 MHz) δ (ppm): 1.01-1.12 (m, 2H, CH₂—CH₂—CH—CHO); 1.24-1.35 (m, 2H, CH₂—CH₂—CH—CHO); 1.49-1.60 (m, 1H, CH); 1.90-1.95 (m, 2H, CH₂—CH₂—CH—CHO); 2.03-2.07 (m, 2H, CH₂—CH₂—CH—CHO); 2.15-2.24 (m, 1H, CHCHO); 3.18 (d, J=6.8 Hz, 2H, CH₂N₃); 9.63 (d, J=1.4 Hz, 1H, CHO). ¹³C NMR (CDCl₃, 90 MHz) δ (ppm): 204.0, 57.5, 50.0, 41.0, 37.3, 29.8, 29.2, 25.3.

The synthesis of trans-4-(4-azidomethylcyclohexylmethyl)-1-(2-methoxyphenyl)piperazine begins by dissolving 0.39 g (2.3 mmol) trans-4-azidomethylcyclohex-1-ylcarbaldehyde and 0.56 g (2.9 mmol) 2-methoxyphenylpiperazine in 15 ml dichlomethane and the addition of 0.74 g (3.5 mmol) sodium triacetoxyborohydride. After 23 hours of reaction at ambient temperature the mixture is washed with NaHCO₃ solution, and the organic phase is concentrated and purified with flash chromatography (EtOAc benzine: 1-1).

Yield: 0.78 g (97%).

IR: (NaCl), 2919, 2851, 2812, 2095, 1500, 1450, 1240. ¹H NMR (CDCl₃, 360 MHz) δ (ppm): 0.87-1.05 (m, 4H, CH₂—CH₂); 1.47-1.50 (m, 2H, CH); 1.80-1.91 (m, 4H, CH₂—CH₂); 2.21 (d, J=7.1 Hz, 2H, CH₂Npip); 2.59 (m, 4H, pip); 3.08 (m, 4H, pip); 3.14 (d, J=36.4 Hz, 2H, CH₂N₃); 3.86 (s, 3H, CH₃O); 6.84-7.01 (m, 4H, phenyl).

The synthesis of trans-4-(4-azidomethylcyclohexylmethyl)-1-(2,3-dichlorophenylpiperazine takes place under identical conditions.

Yield: 0.80 g (85%).

IR: (NaCl): 2930, 2818, 2801, 2096, 1577, 1448. ¹H NMR (CDCl₃, 360 MHz) δ (ppm): 0.87-1.06 (m, 4H, CH—CH₂); 1.44-1.59 (m, 2H, CH); 1.81-1.90 (m, 4H, CH₂—CH₂); 2.21 (d, J=7.1 Hz, 2H, CH₂Npip); 2.57 (m, 4H, pip); 3.05 (m, 4H, pip); 3.14 (d, J=6.4 Hz, 2H, CH₂N₃); 6.92-6.97 (m, 1H, phenyl); 7.10-7.16 (m, 4H, phenyl). ¹³C NMR (CDCl₃, 90 MHz) δ (ppm): 151.4, 134.0, 127.5, 127.4, 124.4, 117.5, 65.4, 58.0, 53.8, 51.4, 38.4, 35.0, 31.1, 30.3.

The amine component trans-4-(4-aminomethylcyclohex-1-ylmethyl)-1-(2-methoxyphenyl)piperazine is produced by preparing a solution of 0.40 g (1.2 mmol) trans-4-(4-azidomethyleyclohexylmethyl)-1-(2-methoxyphenyl)piperazine in 10 ml methanol and the addition of 0.10 g Pd/C 10%. The suspension is agitated under an H₂-atmosphere for 23 hours at ambient temperature. Then the solvent is evaporated in the vacuum and purified with flash chromatography (CH₁₂Cl₂—CH₃OH-NEtMe₂: 90-8-2).

Yield: 0.14 g (39%) (light yellow oil).

MS: 317 m/z (M⁺); IR: (NaCl): 3382, 2912, 2842, 2811, 1500, 1240, 747.11 NMR (CDCl₃, 360 MHz) δ (ppm): 0.87-1.05 (m, 4H, CH₂—CH₂); 1.25-1.30 (m, 1H, CH); 1.45-1.56 (m, 1H, CH); 1.81-1.91 (m, 4H, CH₂—CH₂); 2.21 (d, J=7.1 Hz, 2H, H₂N—CH₂); 2.55 (d, J=6.4 Hz, 2H, CH₂Npip); 2.59 (m, 411, pip); 3.08 (m, 4H, pip); 3.86 (s, 3H, CH₃O); 6.84-7.01 (m, 411, phenyl). ¹³C NMR (CDCl₃, 90 MHz) δ (ppm): 152.3, 141.5, 122.7, 120.9, 118.1, 111.1, 65.7, 55.3, 53.9, 50.7, 48.7, 35.3, 31.4, 30.9, 30.4.

For the production of trans-4-(4-aminomethylcyclohex-1-ylmethyl)-1-(2,3-dichlorophenyl)piperazine 25 ml dry THF 1.05 ml LiAlH₄ solution (1 M in THF) is added to a solution of 0.20 g (0.52 mmol) trans-4-(4-azidomethylcyclohexylmethyl)-1-(2,3-dichlorophen-yl)piperazine and heated for 8 hours with recycling. The solution is evaporated in the vacuum and purified by flash chromatography (CH₂Cl₂—CH₃OH-NEtMe₂: 90-8-2).

Yield: 0.13 g (36%) (light yellow oil).

MS: 355 m/z (M⁺), 357 ((M+2)⁺), 359 ((M+4)⁺); IR: (NaCl): 3375, 2913, 2843, 2817, 1577, 1448, 778. ¹H NMR (CDCl₃, 360 MHz) δ (ppm): 0.85-0.98 (m, 4H, CH₂—CH₂); 1.19-1.31 (m, 1H, CH); 1.43-1.52 (m, 1H, CH); 1.80-1.88 (m, 4H, CH₂—CH₂); 2.19 (d, J=7.1 Hz, 2H, H₂N—CH₂); 2.53-2.56 (m, 6H, pip, CH₂Npip); 3.06-3.08 (m, 3H, pip); 3.17-3.20 (m, 1H, pip); 6.94-6.96 (m, 1H, phenyl), 7.10-7.15 (m, 2H, phenyl).

### SYNTHESIS OF THE EXAMPLE COMPOUNDS

### Example 1

### N-4-(4-(2-methoxyphenyl)piperazin-1-yl)butylindolizin-2-ylcarbamide

0.019 g indolizine-2-carboxylic acid (0.12 mmol) are dissolved in 4 ml dry methylene chloride. 0.07 ml (0.42 mmol) dry DIPEA are added. Then 0.042 g (0.13 mmol) of the TBTU dissolved in 0.5 ml dry DMF are slowly droppered in at 0° C. and agitated for 15 minutes at ambient temperature. The reaction solution is again cooled to 0° C. and a solution of 0.034 g (0.13 mmol) 4-(4-(2-methoxyphenyl)-piperazin-1-yl)butylamine droppered in to 4 ml dry methylene chloride at 0° C. After 1 hour the reaction mixture is absorbed in CH₂CO₂ and washed with saturated NaHCO₃ solution and water. Following drying of the organic phase with MgSO₄ the solvent is evaporated and purified by flash chromatography (SiO₂; CH₂Cl₂—CH₃OH:98-2).

Yield: 39 mg (81%).

M.P.: 143° C.; MS: m/z 406 (M⁺); IR (NaCl): 2933; 2819; 1631; 1558; 1500; 1242; 1029; 750. ¹H NMR (CDCl₃, 360 MHz) δ (ppm): 1.67-1.69 (m, 4H, CH₂—CH₂); 2.50 (t, J=6.9 Hz, 2H, CH₂N); 2.65-2.70 (m, 4H, pip); 3.08-3.11 (m, 4H, pip); 3.47-3.52 (m, 2H, CH₂NHCO); 3.86 (s, 3H, OCH₃); 6.39 (br t, J=5.1 Hz, 1H, NHCO); 6.49-6.53 (m, 1H, H-6); 6.59 (s, 1H, H-1); 6.68 (ddd, J=1.1 Hz, J=6.6 Hz, J=9.1 Hz, 1H, H-7); 6.84-6.87 (m, 1H, H-arom); 7.91-7.02 (m, 3H, H-arom); 7.33 (d, J=9.1 Hz, 1H, H-8); 7.74-7.75 (m, 1H, H-3); 7.87 (dd, J=7.1 Hz, J=0.9 Hz, 1H, H-5). ¹³C NMR (CDCl₃, 90 MHz) δ ppm): 164.9; 152.2; 141.1; 132.8; 125.4; 123.9; 122.9; 120.9; 119.8; 118.2; 113.9; 111.8; 111.2; 97.1; 89.3; 58.1; 55.3; 53.4; 50.4; 39.3; 27.6; 24.2.

CHN (%): C₂₄H₃₀N₄O₂×0.5H₂O

Calculated: C, 69.37; H 7.52; N, 13.48 Actual: C, 69.07; H, 7.30; N, 13.46.

### Example 2

### N-4-(4-(3-chloro-2-methoxyphenyl)piperazin-1-yl)butylindolizin-2-ylcarbamide

Synthesis analogous to example 1.

Yield: 41 mg (75%).

M.P.: 157° C. MS: m/z 440 (M⁺)) 442 ((M+2)⁺), 444 ((M+4)⁺). IR (NaCl): 3321; 2936; 2811; 1626; 1554; 1525; 1250; 1142; 739. ¹H NMR (CDCl₃, 360 MHz) δ (ppm); 1.66-1.68 (m, 4H, CH₂—CH₂); 2.48 (t, J=6.9 Hz, 2H, CH₂Npip); 2.62-2.66 (m, 4H, pip); 3.11-3.18 (m, 4H, pip); 3.47-3.52 (m, 2H, CH₂NHCO); 3.86 (s, 3H, OCH₃); 6.35 (br t, J=5.0 Hz, 1H, NHCO); 6.49-6.53 (m, 1H, H-6); 6.58 (br s, 1H, 1H-1); 6.69 (ddd, J=9.1 Hz, J=6.6 Hz, J=1.1 Hz, 1H, H-7); 6.77 (dd, J=8.0 Hz, J=1.8 Hz, 1H, H-arom); 6.94 (dd, J=8.0 Hz, 1H, H-arom); 7.00 (dd, J=8.0 Hz, J=1.8 Hz, 1H, H-arom); 7.33 (d, J=9.1 Hz, 1H, H-8); 7.74-7.75 (m, 1H, H-3); 7.85 (dd, J=7.1 Hz, J=0.9 Hz, 1H, H-5). ¹³C NMR (CDCl₃, 90 Mhz) δ (ppm): 164.9; 148.6; 146.5; 132.8; 128.7; 125.4; 124.6; 123.9; 123.3; 119.8; 118.2; 117.0; 113.9; 111.8; 97.0; 58.9; 58.1; 53.7; 50.0; 39.4; 27.6; 24.2.

CHN (%): C₂₄H₂₉ClN₄O₂.0.3H₂O

Calculated: C, 64.49; H, 6.69; N, 12.53; Actual: C, 64.57; H, 6.72; N, 12.46.

### Example 3

### N-4-(4-(2,3-dichlorophenyl)piperazin-1-yl)butylindolizin-2-ylcarbamide

Synthesis analogous to example 1.

Yield; 30 mg (57%).

M.P.: 179° C. MS: m/z 444 (M⁺), 446 ((M+2)⁺), 448 ((M+4)). IR (NaCl): 3427; 2925; 2852; 1631; 1529; 1244; 1043; 731. ¹H NMR: (CDCl₃, 360 MHz) δ (ppm): 1.64-1.70 (m, 4H, CH₂—CH₂); 2.50 (t, J=6.9 Hz, 2H, CH₂Npip); 2.63-2.69 (m, 4H, pip); 3.04-3.08 (m, 4H, pip); 3.47-3.52 (m, 2H, CH₂NHCO); 6.33 (br t, J=5.1 Hz, 1H, NHCO); 6.49-6.53 (m, 1H, H-6); 6.58 (s, 1H, H-1); 6.69 (ddd, J=9.1 Hz, J=6.6 Hz, J=1.1 Hz, 1H, H-7); 6.92 (dd, J=7.3 Hz, J=2.3 Hz, 1H, H-arom); 7.10-7.17 (m, 2H, H-arom); 7.33 (d, J=9.1 Hz, 1H, H-8); 7.75-7.76 (m, 1H, H-3); 7.87 (dd, J=7.1 Hz, J=1.0 Hz, 1H, H-5). ¹³C NMR (CDCl₃, 90 MHz) δ (ppm): 164.9; 151.2; 134.0; 132.9; 127.5; 127.4; 125.4; 124.6; 123.9; 119.8; 118.6; 118.3; 113.9; 111.8; 96.9; 58.0; 53.3; 51.1; 39.4; 27.7; 24.3.

### Example 4

### N-4-(4-(2,3-difluorophenyl)piperazin-1-yl)butylindolizin-2-ylcarbamide

Synthesis analogous to example 1.

Yield: 46 mg (93%).

M.P.: 170° C. MS: m/z 412 (M⁺). IR NaCl): 3316; 2946; 2812; 1626; 1556; 1502; 1266; 1142; 767. ¹H NMR (CDCl₃, 360 MHz) δ (ppm): 1.62-1.70 (m, 4H, CH₂—CH₂); 2.46 (t, J=6.9 Hz, 2H, CH₂Npip); 2.61-2.64 (m, 4H, pip); 3.11-3.13 (m, 4H, pip); 3.46-3.52 (m, 2H, CH₂NHCO); 6.29 (br t, J=5.0 Hz, 1H, NHCO); 6.49-6.54 (m, 1H, H-6); 6.57 (s, 1H, H-1); 6.63-6.71 (m, 2H, H-arom, H-7); 6.74-6.80 (m, 1H, H-arom); 6.91-6.98 (m, 1H, H-arom); 7.33 (d, J=9.0 Hz, 1H, H-3); 7.75 (d, J=1.2 Hz, 1H, H-3); 7.85 (dd, J=6.9 Hz, J=1.1 Hz, 1H, H-5). ¹³C NMR (CDCl₃, 90 MHz) δ (ppm); 164.9; 151.5 (dd, ¹J_{C-C}=10.4 Hz; ¹J_{C-F}=244.1 Hz, 1C, PhenylC-2); 143.9 (dd, ¹J_{C-C}=13.9 Hz, ¹J_{C-F}=246.9 Hz, 1C, Phenyl C-3); 141.9 (dd; 3J=5.5 Hz; ⁴J=2.1 Hz, 1C, PhenylC1); 132.9; 125.4; 123.5 (dd, ³J=8.3 Hz, ⁴J=4.9 Hz, 1C, phenylC-4); 123.9; 119.8; 118.2; 117.0; 113.9; 111.8; 109.9; 96.9; 58.0; 53.2; 50.4; 39.4; 27.7; 24.3.

### Example 5

### N-4-(4-(2,3-dihydrobenzofuran-7-yl)piperazin-1-yl)butylindolizin-2-ylcarbamide

Synthesis analogous to example 1.

Yield: 47 mg (94%).

M.P.: 159° C. MS: m/z 418 (M⁺). IR (NaCl): 3323; 2941; 2817; 1634; 1557; 1267; 1146; 753. ¹H NMR (CDCl₃, 360 MHz) δ (ppm): 1.66-1.68 (m, 4H, CH₂—CH₂); 2.48 (t, J=6.9 Hz, 2H, CH₂Npip); 2.67 (m, 4H, pip); 3.17-3.22 (m, 6H, O—CH₂—CH₂, pip); 3.46-3.51 (m, 2H, CH₂NHCO); 4.59 (t, J=8.8 Hz, 2H, O—CH₂—CH₂); 6.38 (br t, J=4.8 Hz, 1H, NHCO); 6.50 (ddd, J=6.9 Hz, J=6.6 Hz, J=1.2 Hz, 1H, H-6); 6.59 (s, 1H, H-1); 6.66-6.70 (m, 2H, H-arom, H-7); 6.77-6.81 (m, 1H, H-arom); 6.86 (dd, J=7.3 Hz, J=1.1 Hz, 1H, H-arom); 7.33 (d, J=9.0 Hz, 1H, H-8); 7.75 (dd, J=2.1 Hz, J=1.1 Hz, 1H, H-3); 7.86 (dd, J=7.1 Hz, J=1.1 Hz, 1H, H-5). ¹³C NMR (CDCl₃, 90 MHz) δ (ppm); 164.9; 151.1; 136.2; 132.8; 127.5; 125.4; 123.9; 121.0; 119.8; 118.2; 118.1; 115.6; 113.9; 111.8; 97.1; 71.0; 58.1; 53.2; 49.3; 39.4; 30.1; 27.6; 24.2.

### Example 10

### N-4-(4-(2-methoxyphenyl)piperazin-1-yl)butyl-3-bromopyrazolo[1,5-a]pyridin-2-ylcarbamide

Synthesis analogous to example 39.

Yield: 149 mg (74% over 2 reaction steps).

M.P.: 108° C. IR (NaCl): 3413, 3326, 2938, 2817, 1668, 1542, 1500, 1240. ¹H NMR (CDCl₃, 360 MHz) δ (ppm): 1.65-1.74 (m, 4H, CH₂—CH₂); 2.45-2.49 (m, 2H, CH₂Npip); 2.67 (m, 4H, pip); 3.10 (m, 4H, pip); 3.50-3.55 (m, 2H, CH₂NHCO); 3.86 (s, 3H, CH₃O); 6.84-7.01 (m, 5H, phenyl, H-6); 7.21-7.25 (m, 1H, H-5); 7.30 (br s, 1H, NHCO); 7.60 (br d, J=8.9 Hz, 1H, H-4); 8.33 (d, J=7.1 Hz, 1H, H-7). ¹³C NMR (CDCl₃, 90 MHz) δ (ppm): 161.0, 152.3, 143.3, 141.4, 140.1, 128.6, 124.6, 122.8, 121.0, 118.2, 118.0, 114.5, 111.2, 85.0, 58.2, 55.3, 53.5, 50.6, 39.2, 27.6, 24.4.

CHN (%):C₂₃H₂₈BrN₅O₂

Calculated: C, 56.79; H, 5.80; N, 14.40; Actual: C, 56.71; H, 5.91; N, 14.44.

### Example 11

### N-4-(4-(2-methoxyphenyl)piperazin-1-yl)butyl-3-chloropyrazolo[1,5-a]pyridin-2-ylcarbamide

Synthesis analogous to example 39.

Yield: 55 mg (61% over 2 reaction steps).

M.P.: 121° C. MS: m/z 441 (M⁺), 443 ((M+2)⁺); IR (NaCl): 3332, 2937, 2815, 1668, 1635, 1545, 1500, 1240. ¹H NMR (CDCl₃, 360 MHz) δ (ppm): 1.65-1.74 (m, 41, CH₂—CH₂); 2.45-2.49 (m, 2H, CH₂Npip); 2.67 (m, 4H, pip); 3.10 (m, 4H, pip); 3.50-3.56 (m, 2H, CH₂NHCO); 3.86 (s, 3H, CH₃O); 6.84-6.89 (m, 1H, H-6); 6.90-7.01 (m, 4H, phenyl); 7.20-7.27 (m, 2H, NHCO, H-5); 7.61 (br d, J=8.9 Hz, 1H, H-4); 8.31 (d, J=7.1 Hz, 1H, H-7). ¹³C NMR (CDCl₃, 90 MHz) δ (ppm): 160.9, 152.3, 141.9, 141.3, 138.6, 128.5, 124.2, 122.8, 121.0, 118.2, 117.2, 114.4, 111.2, 101.0, 58.1, 55.3, 53.4, 50.5, 39.1, 27.6, 24.4.

### Example 12

### N-4-(4-(2-methoxyphenyl)piperazin-1-yl)butyl-5-methylpyrazolo[1,5-a]pyridin-2-ylcarbamide

Synthesis analogous to example 39.

Yield: 60 mg (50% over 2 reaction steps).

M.P.: 119° C. MS: m/z 421 (M⁺), IR (NaCl): 3411, 3336, 2937, 2871, 1662, 1500, 1240.11H NMR (CDCl₃, 360 MHz) δ (ppm): 1.65-1.72 (m, 4H, CH₂—CH₂); 2.34 (s, 3H, CH₃); 2.44-2.48 (m, 2H, CH₂Npip); 2.66 (m, 4H, pip); 3.11 (m, 4H, pip); 3.49-3.54 (m, 2H, CH₂NHCO); 3.86 (s, 3H, CH₃O); 6.66 (d, J=7.1 Hz, 1H, H-6); 6.85-7.01 (m, 5H, phenyl, H-3); 7.25 (br s, 1H, NHCO); 7.33 (s, 1H, H-4); 8.23 (d, J=7.1 Hz, 1H, H-7).

CHN (%): C₂₄H₃₁N₅O₂

Calculated: C. 68.38; H, 7.41; N, 16.61; Actual: C, 67.99; H, 7.51; N, 16.69.

### Example 13

### N-4-(4-(2-methoxyphenyl)piperazin-1-yl)butyl-6-bromopyrazolo[1,5-a]pyridin-2-ylcarbamide

Synthesis analogous to example 39.

Yield: 85 mg (84% over 2 reaction steps).

M.P.: 104° C. MS: 485 m/z (M), 487 ((M+2)⁺); IR (NaCl): 3320, 2937, 2815, 1662, 1552, 1502, 1240. ¹H NMR (CDCl₃, 360 MHz) δ (ppm): 1.65-1.73 (m, 4H, CH₂—CH₂); 2.45-2.49 (m, 2H, CH₂Npip); 2.67 (m, 4H, pip); 3.12 (m, 4H, pip); 3.49-3.55 (m, 2H, CH₂NHCO); 3.86 (s, 3H, CH₃O); 6.84-7.01 (m, 4H, phenyl); 7.07 (s, 1H, H-3); 7.20-7.23 (m, 1H, H-5); 7.28 (br s, 1H, NHCO); 7.49 (d, J=9.6 Hz, 1H, H-4); 8.51 (br s, 1H, H-7). ¹³C NMR (CDCl₃, 90 MHz) δ (ppm): 161.7, 152.3, 148.5, 141.3, 139.7, 128.6, 127.4, 122.9, 120.9, 119.6, 118.1, 111.2, 108.1, 98.8, 58.1, 55.3, 53.4, 50.5, 39.2, 27.6, 24.3.

CHN (%): C₂₃H₂BrN₅O₂

Calculated: C, 56.79; H, 5.80; N, 14.40; Actual: C, 56.39; H, 5.94; N, 14.29.

### Example 14

### N-4-(4-(2-methoxyphenyl)piperazin-1-yl)butyl-3-methoxycarbonylpyrazolo[1,5-a]pyridin-2-ylcarbamide

0.12 g (0.5 mmol) pyrazolo[1,5-a]pyridin-2,3-dicarboxylic acid dimethyl ester, 0.24 g (1.0 mmol) 1-(4-aminobutyl)-4-(2-methoxyphenyl)piperazine and 4.0 mg (0.08 mmol) sodium cyanide are mixed with 2 ml Methanol in a pressure tube and agitated for 62 hours at 50° C. Then the solvent is evaporated in the vacuum in order to obtain the product.

Yield: 0.21 g (89%).

M.P.: 149° C. IR (NaCl): 3286, 2940, 2815, 1683, 1660, 1502, 1444, 1240, 750. ¹H NMR (CDCl₃, 360 MHz) δ (ppm): 1.67-1.77 (m, 4H, CH₂—CH₂); 2.47-2.51 (m, 2H, CH₂Npip); 2.67 (m, 4H, pip); 3.08 (m, 4H, pip); 3.56-3.61 (m, 2H, CH₂NHCO); 3.85 (s, 3H, CH₃O); 4.00 (s, 3H, CH₃O); 6.84-7.01 (m, 4H, phenyl) 7.03-7.07 (m, 1H, H-6), 7.45-7.50 (m, 1H, H-5), 8.15 (br d, J=8.9 Hz, 1H, H-4), 8.64 (br d, J=6.7 Hz, 1H, H-7), 9.98 (br s, 1H, NHCO). ¹³C NMR (CDCl₃, 90 MHz) δ (ppm): 165.4, 160.5, 152.3, 150.1, 142.3, 141.4, 129.7, 128.3, 122.8, 121.0, 120.5, 118.2, 115.1, 111.2, 100.6, 58.3, 55.3, 53.4, 52.2, 50.5, 39.7, 27.3, 24.3.

CHN (%): C₂₅H₃₁N₅O₄.0.5H₂O

Calculated: C, 63.27; H, 6.80; N, 14.76; Actual: C, 62.94; H, 6.73; N, 14.74.

### Example 15

### N-4-(4-(2,3-dichlorophenyl)piperazin-1-yl)butyl-3-methoxycarbonylpyrazolo[1,5-a]pyridin-2-ylcarbamide

Synthesis analogous to example 14 with additional purification by flash chromatography (CH₂Cl₂-MeOH: 95-5).

Yield: 0.20 g (78%).

M.P.: 149° C. MS: m/z 503 (M⁺), 505 ((M+2)⁺), 507 ((M+4)⁺). IR (NaCl): 3471, 3280, 3097, 2944, 2819, 1685, 1660, 1577, 1238. ¹H NMR (CDCl₃, 360 MHz) δ (ppm): 1.67-1.80 (m, 4H, CH₂—CH₂); 2.49 (t, J=7.1 Hz; 2H, CH₂Npip); 2.65 (m, 4H, pip); 3.05 (m, 4H, pip); 3.57-3.62 (m, 2H, CH₂NHCO); 4.01 (s, 3H, CH₃O); 6.91-6.94 (m, 1H, phenyl), 7.04-7.08 (m, 1H, H-6), 7.10-7.16 (m, 2H, phenyl), 7.46-7.51 (m, 1H, H-5), 8.14-8.17 (m, 1H, H-4), 8.63-8.66 (m, 1H, H-7), 10.03 (br s, 1H, NHCO). ¹³ C NMR (CDCl₃, 90 MHz) δ (ppm): 165.5, 160.5, 151.3, 150.0, 142.3, 133.9, 129.7, 128.3, 127.5, 127.4, 124.4, 120.5, 118.5, 115.1, 100.6, 58.2, 53.2, 52.2, 51.3, 39.7, 27.3, 24.3.

CHN (%): C₂₄H₂₇N₅O₃

Calculated: C, 57.15; H, 5.40; N, 13.88; Actual: C, 57.00; H, 5.34; N, 13.86.

### Example 16

### Trans-N-(4-(4-(2-methoxyphenyl)piperazin-1-yl)methylcyclohex-1-ylmethylpyrazolo[1,5-a]pyridin-2-ylcarbamide

0.025 g (0.15 mmol) pyrazolo[1,5-a]pyridine-2-carboxylic acid, 0.026 g (0.17 mmol) HOBt and 0.035 g (0.17 mmol) N,N′-dicyclohexylcarbodiimide are mixed with 3 ml dry methylene chloride and agitated for 0.5 hour at ambient temperature. Then a solution of 0.054 (0.16 mmol) trans-4-(4-aminomethylcyclohex-1-yl)-1-(2-methoxyphenyl)piperazine is droppered into 2.5 ml methylene chloride and agitated for 18 hours at ambient temperature. The resultant solid matter is filtered off and the solution evaporated in the vacuum. Purification is by flash chromatography (CH₂Cl₂-MeOH: 95-5).

Yield: 64 mg (90%).

M.P.: 149° C. MS: m/z 461 (M⁺); IR (NaCl): 3419, 2917, 2845, 2813, 1667, 1635, 1551, 1500, 1240, 734. ¹H NMR (CDCl₃, 360 MHz) δ (ppm): 0.90-1.10 (m, 4H, CH₂-cyclohex); 1.53-1.62 (m, 2H, CH-cyclohex); 1.88 (t, J=10.7 Hz, 4H, CH₂-cyclohex); 2.32 (d, J=6.7 Hz, 2H, CH₂Npip); 2.72 (m, 4H, pip); 3.13 (m, 4H, pip); 3.32-3.36 (m, 2H, CH₂NHCO); 3.85 (s, 3H, CH₃O); 6.82-7.02 (m, 5H, phenyl, H-6), 7.05 (br s, 1H, H-3), 7.13-7.16 (m, 2H, H-5, NHCO), 7.58 (br d, J=8.8 Hz, 1H, H-4), 8.38 (br d, J=7.1 Hz, 1H, H-7). ¹³C NMR (CDCl₃, 90 MHz) δ (ppm): 162.1, 152.2, 148.0, 141.3, 141.1, 128.4, 123.6, 123.0, 121.0, 119.2, 118.2, 113.5, 111.1, 97.9, 65.3, 55.3, 53.7, 50.1, 45.3, 38.3, 34.7, 31.2, 30.4.

### Example 17

### Trans-N-(4-(4-(2,3-dichlorophenyl)piperazin-1-yl)methylcyclohex-1-yl)methylpyrazolo[1,5-a]pyridin-2-ylcarbamide

Synthesis analogous to example 16.

Yield: 13 mg (16%).

M.P.: 138° C. MS: m/z 499 (M⁺), 501 ((M+2)⁺), 503 ((M+4)⁺). IR (NaCl): 2920, 2844, 1669, 1635, 1557, 1448, 1239. ¹H NMR (CDCl₃, 360 MHz) δ (ppm): 0.83-1.11 (m, 4H, CH₂-cyclohex); 1.50-1.61 (m, 2H, CH-cyclohex); 1.85-1.90 (m, 4H, CH₂-cyclohex); 2.17-2.22 (m, 2H, CH₂Npip); 2.50-2.58 (m, 411, pip); 3.05-3.19 (m, 4H, pip); 3.36 (t, J=6.4 Hz, 2H, CH₂NHCO); 6.82-6.87 (m, 1H, H-6); 6.92-6.98 (m, 1H, phenyl), 7.06 (br s, 1H, H-3); 7.10-7.18 (m, 4H, phenyl, H-5, NHCO); 7.59 (br d, J=9.2 Hz, 1H, H-4); 8.38 (br d, J=7.1 Hz, 1H, H-7). ¹³C NMR (CDCl₃, 90 MHz) δ (ppm): 162.1, 151.5, 148.1, 141.4, 134.0, 128.4, 127.4, 124.4, 123.6, 119.3, 118.5, 113.5, 98.0, 91.6, 65.4, 53.7, 51.3, 45.4, 38.5, 35.1, 31.2, 30.5.

CHN (%): C₂₆H₃₁Cl₂N₅O₂

Calculated: C, 62.40; H, 6.24; N, 13-99; Actual: C, 62.55; H, 6.25; N, 13.53.

### Example 22

### Trans-N-(4-(4-(2-methoxyphenyl)piperazin-1-yl)methylyclohex-1-yl)methylpyrazolo[1,5-a]pyridin-3-ylcarbamide

Synthesis analogous to example 16.

Yield: 41 mg (66%).

M.P.: 76° C. MS: m/z 461 (M⁺); IR (NaCl); 3313, 2916, 2844, 2813, 1637, 1627, 1556, 1531, 1499, 1240, 749. ¹H NMR (CDCl₃, 360 MHz) (ppm): 0.89-1.09 (m, 4H, CH₂-cyclohex); 1.50-1.62 (m, 211, CH-cyclohex); 1.88 (t, J=10.8 Hz, 4H, CH₄₂-cyclohex); 2.25 (d, J=7.1 Hz, 2H, CH₂Npip); 2.64 (m, 4H, pip); 3.10 (m, 4H, pip); 3.31-3.35 (m, 2H, CH₂NHCO); 3.85 (s, 3H, CH₃O); 5.94 (m, 1H, NHCO); 6.84-7.01 (m, 5H, phenyl, H-6), 7.32-7.37 (m, 1H, H-5), 8.14 (br s, 1H, H-2), 8.31 (br d, J=8.8 Hz, 1H, H-4), 8.48 (br d, J=7.1 Hz, 1H, H-7). ¹³C NMR (CDCl₃, 90 MHz) δ (ppm): 163.3, 152.3, 141.4, 140.6, 140.1, 128.8, 126.3, 122.8, 121.0, 119.7, 118.2, 113.5, 111.1, 106.9, 65.4, 55.3, 53.9, 50.5, 45.5, 38.5, 35.0, 31.2, 30.6.

### Example 23

### Trans-N-(4-(4-(2,3-dichlorophenyl)piperazin-1-yl)methylcyclohex-j-yl)methylpyrazolo[1,5-a]pyridin-3-ylcarbamide

Synthesis analogous to example 16.

Yield: 42 mg (51%).

M.P.: 68° C. MS: m/z 499 (M⁺), 501 ((M+2)⁺), 503 ((M+4)⁺). IR (NaCl): 3308, 2920, 2847, 1637, 155, 1530, 1449, 1272, 1240, 745. ¹H NMR (CDCl₃, 360 MHz) δ (ppm); 0.88-110 (m, 4H, CH₂-cyclohex); 1.45-1.61 (m, 2H, CH-cyclohex); 1.87-1.91 (m, 4H, CH₂-cyclohex); 2.17-2.23 (m, 2H, CH₂Npip); 2.54-2.58 (m, 4H, pip); 3.06-3.19 (m, 4H, pip); 3.32-3.36 (m, 2H, CH₂NHCO); 5.90 (s, 1H, NHCO); 6.82-6.87 (m, 1H, H-6); 6.90-6.97 (m, 14, phenyl), 7.11-7.18 (m, 2H, phenyl); 7.34-7.37 (m, 1H H-5); 8.13 (s, 1H, H-2); 8.32 (br d, J=8.9 Hz, 1H, H-4); 8.48 (br d, J=7.1 Hz, 1H, H-7). ¹³C NMR (CDCl₃, 90 MHz) δ (ppm): 163.3, 151.4, 140.6, 140.1, 133.6, 128.8, 127.4, 126.3, 124.4, 123.5, 119.0, 115.9, 113.6, 106.9, 65.4, 53.7, 51.3, 45.5, 38.6, 35.1, 31.2, 30.6.

### Example 28

### N-4-(4-(2-methoxyphenyl)piperazin-1-yl)butyl-3-bromopyrazolo[1,5-a]pyridin-5-ylcarbamide

Synthesis analogous to example 39.

Yield: 59 mg (59% over 2 reaction steps).

M.P.: 172° C. IR (NaCl): 3316, 2939, 2817, 1648, 1546, 1500, 1240. ¹H NMR (CDCl₃, 360 MHz) δ (ppm): 1.69-1.78 (m, 4H, CH₂—CH₂); 2.49-2.53 (m, 24, CH₂Npip); 2.68 (m, 4H, pip); 3.04 (m, 4H, pip); 3.50-3.55 (m, 2H, CH₂NHCO); 3.84 (s, 31, CH₃O); 6.76-7.01 (m, 4H, phenyl); 7.20-7.25 (m, 2H, NHCO, 4-6); 7.88 (s, 1H, H-4); 7.98 (s, 1H, H-2); 8.42-8.44 (m, 1H, H-7). ¹³C NMR (CDCl₃, 90 MHz) δ (ppm): 165.4, 152.2, 142.7, 140.9, 137.1, 131.2, 129.1, 123.0, 120.9, 118.1, 115.5, 111.1, 111.0, 86.4, 58.0, 55.3, 53.4, 50.4, 40.2, 27.3, 24.4.

CHN (%): C₂₃H₂₈BrN₅O₂

Calculated: C, 56.79; H, 5.80; N, 14.40; Actual: C, 56.67; H, 5.86; N, 14.21.

### Example 29

### N-4-(4-(2-methoxyphenyl)piperazin-1-yl)butyl-3-chloropyrazolo[1,5-a]pyridin-5-ylcarbamide

Synthesis analogous to example 39.

Yield: 62 mg (54% over 2 reaction steps).

M.P.: 155° C. MS: m/z 441 (M), 443 ((M+2)⁺); IR (NaCl): 3307, 2940, 2817, 1647, 1546, 1500, 1240. ¹H NMR (CDCl₃, 360 MHz) δ (ppm): 1.67-1.77 (m, 4H, CH₂—CH₂); 2.49-2.52 (m, 2H, CH₂Npip); 2.67 (m, 4H, pip); 3.04 (m, 4H, pip); 3.49-3.54 (m, 24, CH₂NHCO); 3.84 (s, 3H, CH₃O); 6.77-7.01 (m, 4H, phenyl); 7.21 (d, J=7.4 Hz, 1H, H-6); 7.18-7.22 (m, 1H, NHCO); 7.91 (s, 1H, H-4); 7.95 (s, 1H, H-2); 8.40 (d, J=7.4 Hz, 1H, H-7). ¹³C NMR (CDCl₃, 90 MHz) δ (ppm): 165.4, 152.2, 141.0, 140.6, 135.6, 130.7, 129.1, 123.0, 121.0, 118.1, 114.9, 111.2, 110.9, 102.5, 58.0, 55.3, 53.5, 50.4, 40.2, 27.4, 24.5.

CHN (%): C₂₃H₂₈ClN₅O₂.0.2H₂O

Calculated: C, 62.00; H, 6.42; N, 15.72; Actual: C, 61.66; H, 6.41; N, 15.72.

### Example 30

### N-5-(4-(2-methoxyphenyl)piperazin-1-yl)pentylpyrazolo[1,5-a]pyridin-5-ylcarbamide

Synthesis analogous to example 16.

Yield: 63 mg (85%) (waxlike). IR NaCl): 3309, 2938, 2832, 1650, 1546, 1502, 1243. ¹H NMR (CDCl₃, 360 MHz) δ (ppm): 1.42-1.50 (m, 2H, CH₂—CH₂—CH₂); 1.58-1.73 (m, 4H, CH₂—CH₂—CH₂); 2.45 (t, J=7.45 Hz, 2H, CH₂Npip); 2.68 (m, 4H, pip); 3.12 (m, 4H, pip); 3.47-3.52 (m, 2H, CH₂NHCO); 3.86 (s, 3H, CH₃O); 6.35 (br s, 1H, NHCO); 6.67 (br d, J=2.1 Hz, 1H, H-3); 6.85-7.02 (m, 4H, phenyl); 7.12 (br d, J=7.1 Hz, 1H, 1-6), 8.01 (s, 1H, H-4), 8.01 (d, J=2.1 Hz, 1H, H-2), 8.49 (br d, J=7.1 Hz, 1H, H-7). ³C NMR (CDCl₃, 90 MHz) δ (ppm): 165.6, 152.2, 142.8, 141.2, 139.0, 129.7, 128.6, 122.9, 121.0, 118.2, 117.4, 111.1, 109.6, 99.3, 58.4, 55.3, 53.4, 50.4, 40.1, 29.4, 26.3, 24.8.

### Example 39

### N-4-(4-(2-methoxyphenyl)piperazin-1-yl)butyl-4,5,6,7-tetrahydropyrazolo[1,5-a]pyridin-2-ylcarbamide

0.050 g (0.3 mmol) 4,5,6,7-tetrahydropyrazolo[1,5-a]pyridine-2-carboxylic acid are dissolved in 2 ml dry toluene. 80 μl (0.9 mmol) oxalyl chloride are added and heated at 40° C. until gas starts to form. Then agitation takes place initially for 1 hour at ambient temperature, and then for 3.5 hours at 60° C. The solvent is evaporated in the vacuum and 2 ml abs. methylene chloride are added to the residue. The acid chloride is added under agitation at 0° C. to a solution of 0.36 mmol 4-(4-aminobutyl)-1-(2-methoxyphenyl)piperazine (0.095 g) in 2 ml abs. methylene chloride. The solution is slowly heated to ambient temperature and agitated for 1 hour. Following addition of NaHCO₃ solution extraction is performed with methylene chloride, the organic phase is dried with MgSO₄ and evaporated in the vacuum. Purification takes place by flash chromatography on silica gel (CH₂Cl₂-MeOH:95-5).

Yield; 93 mg (75% over 2 reaction steps).

M.P.: 62° C. MS: 411 m/z (M⁺); IR (NaCl): 3355, 2929, 2852, 1662, 1531, 1240. ¹H NMR (CDCl₃, 360 MHz) δ ppm): 1.61-1.65 (m, 4H, CH₂—CH₂); 1.83-1.90 (m, 2H, H-5); 2.01-2.08 (m, 2H, H-6); 2.44 (t, J=6.7 Hz, 2H, CH₂Npip); 2.65 (m, 4H, pip); 2.81 (t, J=6.4 Hz, 2H, H-4); 3.10 (m, 4H, pip); 3.41-3.47 (m, 2H, CH₂NHCO); 3.86 (s, 3H, CH₃O); 4.09-4.12 (m, 2H, H-7); 6.49 (s, 1H, H-3), 6.84-7.00 (m, 4H, phenyl). ¹³C NMR (CDCl₃, 90 MHz) δ (ppm): 162.4, 152.3, 145.9, 141.4, 140.7, 122.8, 121.0, 118.2, 111.2, 103.6, 58.2, 55.3, 53.4, 50.6, 48.2, 38.9, 27.7, 24.4, 23.3, 22.6, 20.3.

### Example 40

### (±)-N-4-(4-(2-methoxyphenyl)piperazin-1-yl)butyl-5-methyl-4,5,6,7-tetrahydropyrazolo[1,5-a]pyridin-2-ylcarbamide

Synthesis analogous to example 39.

Yield: 107 mg (91% over 2 reaction steps).

M.P.: 65° C. MS: 425 m/z (M⁺); IR (NaCl): 3343, 2937, 2815, 1662, 1533, 1500, 1240. ¹H NMR (CDCl₃, 360 MHz) δ (ppm): 1.12 (d, 3H, J=6.4 Hz, CH₃); 1.60-1.67 (m, 41, CH₂—CH₂); 1.68-1.78 (m, 1H, H-5); 1.96-2.08 (m, 2H, H-6); 2.36 (dd, J=16.3 Hz, J=10.3 Hz, 1H, H-4); 2.44 (t, J=6.7 Hz, 2H, CH₂Npip); 2.65 (m, 4H, pip); 2.93 (dd, J=16.3 Hz, J=5.0 Hz, 1H, H-4); 3.10 (m, 4H, pip); 3.41-3.46 (m, 2H, CH₂NHCO); 3.86 (s, 3H, CH₃O); 3.98-4.06 (m, 1H, H-7); 4.19-4.25 (m, 1H, H-7); 6.47 (s, 1H, H-3), 6.84-7.01 (m, 4H, phenyl). ¹³C NMR (CDCl₃, 90 MHz) δ (ppm): 162.4, 152.3, 146.2, 141.4, 140.7, 122.8, 120.9, 118.2, 111.2, 103.5, 58.2, 55.3, 53.4, 50.6, 47.5, 38.9, 31.2, 30.8, 27.7, 27.1, 24.3, 20.8.

### Example 49

### N-4-(chroman-8-yl)piperazin-1-yl)butylindolizin-2-ylcarbamide

Synthesis analogous to example 1.

Yield: 30 mg (69%).

M.P.: 75° C. MS: m/z 432 (M⁺). IR (NaCl): 3321; 2935; 2873; 2817; 1636; 1558; 1266; 1143; 754. ¹H NMR (CDCl₃, 360 MHz) δ (ppm): 1.67-1.69 (m, 4H, CH₂—CH₂); 1.96-2.03 (m, 2H, O—CH₂—CH₂—CH₂); 2.51-2.55 (m, 2H, CH₂Npip); 2.70-2.74 (m, 4H, pip); 2.77-2.80 (m, 2H, O—CH₂—CH₂—CH₂); 3.10-3.14 (m, 4H, pip); 3.46-3.51 (m, 2H, CHkNHCO); 4.23-4.26 (m, 2H, O—CH₂—CH₂—CH₂); 6.38 (br t, J=4.1 Hz, 1H, NHCO); 6.51 (ddd, J=6.9 Hz, J=6.6 Hz, J=1.2 Hz, 1H, H-6); 6.59 (s, 1H, H-1); 6.68 (ddd, 1H, J=9.0 Hz, J=6.6 Hz, J=0.9 Hz, H-7); 6.71-6.80 (m, 3H, 14-arom); 7.32 (d, J=9.0 Hz, 1H, H-8); 7.76 (d, J=1.1 Hz, 1H, H-3); 7.86 (dd, J=7.1 Hz, J=1.1 Hz, 1H, H-5). ¹³C NMR (CDCl₃, 90 MHz) δ (ppm): 164.9; 147.6; 140.6; 132.8; 125.4; 124.0; 123.9; 122.7; 119.9; 119.8; 118.2; 115.9; 113.9; 111.8; 97.2; 66.5; 58.0; 53.4; 50.3; 39.2; 27.5; 25.1; 24.0; 22.7; 21.0.

### Example 69

### N-4-(4-(2-methoxyphenyl)piperazin-1-yl)butylindolizin-1-ylcarbamide

For the synthesis 0.039 g indolizine-1-carboxylic acid (0.24 mmol) are dissolved in 6 ml dry methylene chloride. Then 0.14 ml (0.84 mmol) dry DIPEA are added and subsequently 0.084 g (0.26 mmol) of the TBTU dissolved in 0.5 ml dry DMF at 0° C. are slowly droppered in and agitation is performed for 30 minutes at ambient temperature. The reaction solution is again cooled to 0° C. and a solution of 0.071 g (0.27 mmol) 4-(4-(2-methoxyphenyl)piperazin-1-yl)butylamine in 4 ml dry methylene chloride is droppered in. After 1 hour of agitation at 0° C. the reaction deposit is diluted with methylene chloride and washed with saturated NaHCO₃ solution and water. Following drying of the organic phase with MgSO₄ the solvent is evaporated and purified by flash chromatography (SiO₂; CH₂Cl₂—CH₃OH:98-2).

Yield: 59 mg (61%) colourless solid matter.

M.P.: 54-56° C. MS: m/z 407 (M⁺). IR (NaCl): 3414; 3339; 2934; 2817; 1634; 1500; 1241; 1028; 749. ¹H NMR (CDCl₃, 360 MHz) δ (ppm): 1.64-1.72 (m, 4H, CH₂—CH₂); 2.52-2.54 (m, 2H, CH₂N); 2.68-2.76 (m, 4H, pip); 3.06-3.16 (m, 4H, pip); 3.51-3.56 (m, 2H, CH₂NHCO); 6.08-6.14 (m, 1H, NHCO); 6.61-6.66 (m, 1H); 6.85-6.87 (m, 1H); 6.90-7.02 (m, 5H, H-arom); 7.20 (d, J=2.7 Hz, 1H); 7.93 (d, J=7.2 Hz, 1H); 8.32 (d, J=8.9 Hz, 1H).

### Example 70

### N-4-(4-(2-methoxyphenyl)piperazin-1-yl)butyl-5,6,7,8-tetrahydroindolizin-2-ylcarbamide

0.020 g tetrahydroindolizine-2-carboxylic acid (0.12 mmol) are converted as described for example 69 and purified by flash chromatography (SiO₂; CH₂Cl₂—CH₃OH:98-2).

Yield: 29 mg (59%).

M.P.: 51-53° C.; MS: m/z 411 (M⁺); IR (NaCl): 3325; 2938; 2817; 1629; 1500; 1241; 1028; 750. ¹H NMR (CDCl₃, 360 MHz) δ (ppm): 1.59-1.66 (m, 4H, CH₂—CH₂); 1.76-1.83 (m, 21H, H-7); 1.88-1.97 (m, 2H, H-6); 2.44 (t, J=6.8 Hz, 2H, CH₂N); 2.62-2.69 (m, 4H, pip); 2.73 (dd, J=6.2 Hz, 2H, H-8); 3.07-3.13 (m, 4H, pip); 3.38-3.43 (m, 2H, CH₂NHCO); 3.86 (s, 3H, OCH₃); 3.92 (dd, J=6.0 Hz, 2H, H-5); 5.88 (br t, J=5.4 Hz, 1H, NHCO); 6.00 (dd, J=0.9 Hz, J=0.9 Hz, 1H, H-1); 6.83-7.01 (m, 4H, H-arom); 7.05 (d, J=1.8 Hz, 1H, H-3).

### Example 71

### N-4-(4-(2,3-dichlorophenyl)piperazin-1-yl)butyl-5,6,7,8-terahydroindolizin-2-ylcarbamide

0.040 g tetrahydroindolizine-2-carboxylic acid (0.24 mmol) are converted as described for example 69 and purified by flash chromatography (SiO₂; CH₂Cl₂—CH₃OH:98-2).

Yield: 49 mg (45%).

M.P.: 64-66° C.; MS: m/z 448 (M⁺), 450 (M⁺+2); IR (NaCl): 3329; 2940; 2863; 2822; 1627; 1243; 755. ¹H NMR (CDCl₃, 360 MHz) δ (ppm): 1.64-1.71 (m, 4H, CH₂—CH₂); 1.81-1.85 (m, 2H, H-7); 1.93-1.97 (m, 2H, H-6); 2.64 (t, J=6.8 Hz, 2H, CH₂N); 2.75 (dd, J=6.4 Hz, 2H, H-8); 2.78-2.85 (m, 4H, pip); 3.13-3.17 (m, 4H, pip); 3.42-3.46 (m, 2H, CH₂NHCO); 3.94 (dd, J=6.0 Hz, 2H, H-5); 6.03 (br t, J=5.3 Hz, 1H, NHCO); 6.06 (br s, 1H, H-1); 6.98 (dd, J=1.7 Hz, J=7.7 Hz, 1H, H-arom); 7.09 (d, J=1.9 Hz, 1H, H-3); 7.15-7.20 (m, 2H, H-arom).

### Example 72

### N-4-(4-(2-methoxyphenyl)piperazin-1-yl)butyl-1-cyano-2-methyl-indolizin-3-ylcarbamide

The 1-cyano-2-methylindolizine-3-carboxylic acid (0.031 g (0.19 mmol)) obtained according to method A7 is converted as described for example 69 and purified by flash chromatography (SiO₂; CH₂Cl₂—CH₃OH:98-2).

Yield: 60 mg (71%).

M.P.: 63-65° C.; MS: m/z 445 (M⁺); IR (NaCl): 3347; 2939; 2817; 2211; 1638; 1512; 1500; 1241; 1027; 750. ¹H NMR (CDCl₃, 360 MHz) δ (ppm): 1.64-1.76 (m, 4H, CH₂—CH₂); 2.48 (t, J=6.9 Hz, 2H, CH₂N); 2.59-2.69 (m, 4H, pip); 2.63 (s, 3H, CH₃); 2.98-3.07 (m, 4H, pip); 3.49-3.56 (m, 2H, CH₂NHCO); 3.85 (s, 3H, OCH₃); 6.29 (br t, J=3.5 Hz, 1H, NHCO); 6.83-6.93 (m, 4H, H-arom, H-6); 6.96-7.01 (m, 1H, H-arom); 7.20 (ddd, J=1.0 Hz, J=6.8 Hz, J=8.9 Hz, 1H, H-7); 7.59 (ddd, J=31.2 Hz, J=31.2 Hz, J=8.9 Hz, 1H, H-8); 9.34 (ddd, 3=1.0 Hz, J=1.0 Hz, J=7.2 Hz, 1H, H-5).

### Example 73

### N-4-(4-phenylpiperazin-1-yl)butylpyrazolo[1,5-a]pyridin-2-ylcarbamide

0.14 ml (0.84 mmol) dry DIPEA are added to 0.039 g pyrazolo[1,5-a]pyridine-2-carboxylic acid (0.24 mmol) dissolved in 6 ml dry methylene chloride. Then 0.042 g (0.13 mmol) TBTU dissolved in 0.5 ml dry DMF are slowly droppered in at 0° C. and agitated for 30 minutes at ambient temperature. The reaction solution is again cooled to 0° C. and a solution of 0.065 g (0.28 mmol) 4-(4-phenylpiperazin-1-yl)butylamine (produced according to the specifications for type C2 amines) are droppered into 4 ml dry methylene chloride. After 1 hour the reaction deposit is diluted with methylene chloride and washed with saturated NaHCO₃ solution and water. Following drying of the organic phase with MgSO₄ the solvent is evaporated and purified by flash chromatography (SiO₂; CH₂Cl₂—CH₃OH:98-2).

Yield: 64 mg (71%) white solid matter.

M.P.: 164-166° C.; MS: m/z 377 (M⁺); IR (NaCl): 3380; 2936; 2819; 1655; 1633; 1547; 1503; 1241; 764; 749. ¹H NMR (CDCl₃, 360 MHz) δ (ppm): 1.68-1.75 (m, 4H, CH₂—CH₂); 2.47 (t, J=7.0 Hz, 2H, CH₂N); 2.61-2.65 (m, 4H, pip); 3.20-3.25 (m, 4H, pip); 3.50-3.55 (m, 2H, CH₂NHCO); 6.81-6.87 (m, 2H, H-arom, H-6); 6.91-6.94 (m, 2H, H-arom); 7.05 (d, J=0.9 Hz, 1H, H-3); 7.13 (ddd, J=1.0 Hz, J=6.7 Hz, J=8.9 Hz, 1H, H-5); 7.22-7.28 (m, 3H, H-arom, NHCO); 7.58 (br d, J=9.0 Hz, 1H, H-4); 8.34 (br d, J=1.0 Hz, J=7.2 Hz, 1H, H-7).

### Example 74

### N-4-(4-(2-methylphenyl)piperazin-1-yl)butylpyrazolo[1,5-a]pyridin-2-ylcarbamide

Synthesis analogous to example 73.

Yield: 75 mg (80%) yellow solid matter.

M.P.: 99-101° C.; MS: m/z 391 (M⁺); IR (NaCl): 3412; 2937; 2855; 2812; 1663; 1552; 1492; 1227; 1042; 764. ¹H NMR (CDCl₃, 360 MHz) δ (ppm): 1.66-1.77 (m, 4H, CH₂—CH₂); 2.32 (s, 3H, CH₃); 2.50 (t, J=7.0 Hz, 2H, CH₂N); 2.56-2.73 (m, 4H, pip); 2.94-3.03 (m, 4H, pip); 3.53-3.60 (m, 2H, CH₂NHCO); 6.87 (dd, J=6.2 Hz, J=6.2 Hz, 1H, H-6); 6.99 (dd, J=7.2 Hz, J=7.2 Hz, 1H, H-5); 7.03-7.05 (m, 1H, H-arom); 7.07 (br s, 1H, H-3); 7.14-7.21 (m, 3H, H-arom); 7.31 (br t, J=5.7 Hz, 1H, NHCO); 7.61 (br d, J=9.0 Hz, 1H, H-4); 8.39 (br d, J=6.8 Hz, 1H, H-7). ¹³C NMR (CDCl₃, 90 MHz) δ (ppm): 162.2; 151.5; 148.1; 141.3; 132.6; 131.0; 128.4; 126.5; 123.6; 123.1; 119.3; 119.0; 113.5; 97.9; 58.1; 53.4; 51.7; 39.1; 27.6; 24.3; 17.8.

### Example 75

### N-4-(4-(2-biphenyl)piperazin-1-yl)butylpyrazolo[1,5-a]pyridin-2-ylcarbamide

Synthesis analogous to example 73.

Yield: 83 mg (71%) colourless oil.

MS: m/z 454 (M⁺). IR (NaCl): 3412; 3331; 2939; 2814; 1665; 1635; 1551; 1225; 1146; 1045; 741. ¹H NMR (CDCl₃, 360 MHz) δ ppm): 1.56-1.68 (m, 4H, CH₂—CH₂); 2.36-2.39 (m, 6H, CH₂N, pip); 2.86-2.89 (m, 41, pip); 3.46-3.50 (m, 2H, CH₂NHCO); 6.84 (ddd, J=1.1 Hz, J=6.8 Hz, J=6.8 Hz, 1H, H-6); 7.01-7.07 (m, 3H, H-arom, H-3); 7.13 (dd, J=6.8 Hz, J=8.9 Hz, 1H, H-5); 7.22-7.29 (m, 4H, H-arom 1, NHCO); 7.37-7.40 (m, 2H, H-arom); 7.58 (d, J=8.9 Hz, 1H, H-4); 7.61-7.63 (m, 2H, H-arom); 8.34 (d, J=6.4 Hz, 1H, H-7).

### Example 76

### N-4-(4-(2-ethoxyphenyl)piperazin-1-yl)butylpyrazolo[1,5-a]pyridin-2-ylcarbamide

Synthesis analogous to example 73.

Yield: 24 mg (47%) yellow oil.

M.P.: 118-120° C.; MS: m/z 422 (M); IR (NaCl): 3411; 2935; 2815; 1663; 1552; 1500; 1241; 1043; 748. ¹H NMR (CDCl₃, 360 MHz) δ (ppm): 1.45 (t, J=6.9 Hz, 3H, O—CH₂—CH₃); 1.66-1.75 (m, 4H, CH₂—CH₂); 2.53 (t, J=6.4 Hz, 2H, CH₂N); 2.68-2.76 (m, 4H, pip); 3.09-3.22 (m, 4H, pip); 3.50-3.56 (m, 2H, CH₂NHCO); 4.06 (q, J=6.9 Hz, 2H, O—CH₂—CH₃); 6.82-6.86 (m, 2H, H-arom); 6.88-6.92 (m, 2H, H-arom, H-6); 6.94-6.98 (m, 1H, H-arom); 7.05 (br s, 1H, H-3); 7.12-7.15 (m, 1H, H-5); 7.30 (br t, J=4.2 Hz, 1H, NHCO); 7.58 (br d, J=9.1 Hz, 1H, H-4); 8.36 (d, J=6.8 Hz, 1H, H-7). ¹³C NMR (CDCl₃, 90 MHz) δ (ppm): 162.2; 151.6; 148.1; 141.3; 141.2; 128.4; 123.6; 122.8; 121.0; 119.2; 118.2; 113.5; 112.6; 97.9; 63.6; 58.2; 53.5; 50.3; 39.1; 27.6; 24.1; 14.9.

### Example 77

### N-4-(4-(2-benzyloxyphenyl)piperazin-1-yl)butylpyrazolo[1,5-a]pyridin-2-ylcarbamide

Synthesis analogous to example 73.

Yield: 86 mg (74%) colourless oil.

MS: m/z 483 (M⁺). IR (NaCl): 3411; 2934; 2814; 1664; 1635; 1551; 1241; 1146; 1016; 750. ¹H NMR (CDCl₃, 360 MHz) δ (ppm): 1.64-1.74 (m, 4H, CH₂—CH₂); 2.46 (Br t, J=6.9 Hz, 2H, CH₂N); 2.61-2.67 (m, 4H, pip); 3.13-3.21 (m, 4H, pip); 3.50-3.55 (m, 2H, CH₂NHCO); 5.13 (s, 2H, —CH₂—O); 6.83 (ddd, J=1.3 Hz, J=6.9 Hz, J=6.9 Hz, 1H, H-6); 6.93-7.96 (m, 4H, H-arom-CH₂); 7.05 (d, J=0.9 Hz, 1H, H-3); 7.13 (ddd, J=1.0 Hz, J=6.7 Hz, J=8.9 Hz, 1H, H-5); 7.25-7.33 (m, 2H, H-arom-CH₂, NHCO); 7.36-7.40 (m, 2H, H-arom); 7.43-7.46 (m, 2H, H-arom); 7.58 (d, J=8.9 Hz, 1H, H-4); 8.35 (dd, J=1.1 Hz, J=7.0 Hz, 1H, H-7).

### Example 78

### N-4-(4-(2-methylmercaptophenylppiperazin-1-yl)butylpyrazolo[1,5-a]pyridin-2-ylcarbamide

Synthesis analogous to example 73.

Yield: 72 mg (71%) colourless oil.

M.P.: 50-52° C.; MS: m/z 423 (M⁺); IR (NaCl): 3412; 2940; 2816; 1664; 1636; 1552; 1519; 1259; 119; 1046; 752. ¹H NMR (CDCl₃, 360 MHz) δ (ppm): 1.66-1.75 (m, 4H, CH₂—CH₂); 2.41 (s, 3H, CH₃); 2.55 (t, J=6.9 Hz, 2H, CH₂N); 2.68-2.77 (m, 4H, pip); 3.03-3.10 (m, 4H, pip); 3.51-3.56 (m, 2H, CH₂NHCO); 6.84 (ddd, J=1.4 Hz, J=6.8 Hz, J=6.8 Hz, 1H, H-6); 7.03-7.06 (m 1H, H-arom); 7.05 (d, J=0.7 Hz, 1H, H-3); 7.07-7.12 (m, 3H, H-arom); 7.13 (ddd, J=1.1 Hz, J=6.8 Hz, J=8.9 Hz, 1H, H-5); 7.30 (br t, J=5.0 Hz, 1H, NHCO); 7.58 (d, J=8.9 Hz, 1H, H-4); 8.36 (dd, J=1.0 Hz, J=7.2 Hz, 1H, H-7).

### Example 79

### N-4-(4-(2-fluorophenyl)piperazin-1-yl)butylpyrazolo[1,5-a]pyridin-2-ylcarbamide

Synthesis analogous to example 73.

Yield: 76 mg (80%) white solid matter.

M.P.: 98-100° C.; MS; m/z 395 (M⁺); IR (NaCl): 3414; 2940; 2853; 2819; 1664; 1636; 1552; 1501; 1239; 1039; 753. ¹H NMR (CDCl₃, 360 MHz) δ ppm): 1.61-1.78 (m, 4H, CH₂—CH₂); 2.47 (t, J=7.0 Hz, 2H, CH₂N); 2.61-2.67 (m; 4H, pip); 3.10-3.16 (m, 4H, pip); 3.50-3.56 (m, 2H, CH₂NHCO); 6.84 (ddd, J=1.4 Hz, J=6.9 Hz, J=6.9 Hz, 1H, H-6); 6.87-7.07 (m, 4H, H-arom); 7.05 (d, J=0.7 Hz, 1H, H-3); 7.13 (ddd, J=1.1 Hz, J=6.8 Hz, J=8.9 Hz, 1H, H-5); 7.28 (br t, J=5.9 Hz, 1H, NHCO); 7.58 (br d, J=8.9 Hz, 1H, H-4); 8.35 (br dd, J=1.1 Hz, J=7.0 Hz, 1H, H-7). ¹³C NMR (CDCl₃, 90 MHz) δ (ppm): 162.1; 155.8 (d, J=245, fluorophenyl); 148.1; 141.3; 140.2 (d, J=8.6 Hz, fluorophenyl); 128.4; 124.4 (d, J=4.0 Hz, fluorophenyl); 123.6; 122.3 (d, J=7.9 Hz, fluorophenyl); 119.2; 118.5 (d, J=3.3 Hz, fluorophenyl); 116.1 (d, J=21 Hz, fluorophenyl); 113.5; 97.9; 58.1; 55.6; 53.4; 50.5; 39.1; 27.6; 24.3.

### Example 80

### N-4-(4-(2-trifluoromethylphenyl)piperazin-1-yl)butylpyrazolo[1,5-a]pyridin-2-ylcarbamide

Synthesis analogous to example 73.

Yield: 83 mg (78%) colourless oil.

MS: m/z 445 (M⁺). IR (NaCl): 3414; 3337; 2939; 2817; 1656; 1636; 1553; 1259; 1140; 1036; 766. ¹H NMR (CDCl₃, 360 MHz) δ (ppm): 1.67-1.74 (m, 4H, CH₂—CH₂); 2.52 (br t, J=6.7 Hz, 2H, CH₂N); 2.55-2.60 (m, 4H, pip); 2.98-3.03 (m, 4H, pip); 3.51-3.56 (m, 2H, CH₂NHCO); 6.84 (ddd, J=1.4 Hz, J=6.9 Hz, J=6.9 Hz, 1H, H-6); 7.05 (d, J=0.7 Hz, 1H, H-3); 7.14 (ddd, J=1.1 Hz, J=6.8 Hz, J=8.9 Hz, 1H, H-5); 7.18-7.23 (m, 1H, 14-arom); 7.29 (br t, J=4.7 Hz, 1H, NHCO); 7.35-7.38 (m 1H, H-arom); 7.47-7.52 (m, 1H, 1-arom 1); 7.57-7.63 (m, 2H, H-arom, H-4); 8.36 (dd, J=0.9 Hz, J=7.0 Hz, 1H, H-7).

### Example 81

### N-4-(4-(2-cyanophenyl)piperazin-1-yl)butylpyrazolo[1,5-a]pyridin-2-ylcarbamide

Synthesis analogous to example 73, wherein the amine component 4-(4-phenylpiperazin-1-yl)butylamine was produced according to the specifications for type C1 amines.

Yield: 62 mg (64%) colourless solid matter.

M.P.: 144-146° C. MS: m/z 402 (M⁺). IR (NaCl): 3411; 2933; 2818; 2219; 1662; 1635; 1553; 1515; 1258; 1144; 1038; 761. ¹H NMR (CDCl₃, 360 MHz) δ (ppm): 1.62-1.75 (m, 4H, CH₂—CH₂); 2.49 (br t, J=7.0 Hz, 2H, CH₂N); 2.66-2.69 (m, 4H, pip); 3.24-3.27 (m, 4H, pip); 3.50-3.56 (m, 2H, CH₂NHCO); 6.84 (ddd, J=1.2 Hz, J=6.9 Hz, J=6.9 Hz, 1H, H-6); 6.96-7.01 (m, 2H, H-arom); 7.05 (d, J=0.9 Hz, 1H, H-3); 7.14 (ddd, J=1.0 Hz, J=6.7 Hz, J=8.9 Hz, 1H, H-5); 7.27 (br t, J=3.9 Hz, 1H, NHCO); 7.44-7.49 (m 1H, phenyl); 7.54-7.57 (m, 1H, H-arom); 7.58 (d, J=8.9 Hz, 1H, H-4); 8.36 (dd, J=0.9 Hz, J=7.0 Hz, 1H, H-7).

### Example 82

### N-4-(4-(2-nitrophenyl)piperazin-1-yl)butylpyrazolo[1,5-a]pyridin-2-ylcarbamide

Synthesis analogous to example 73.

Yield: 61 mg (60%) orange oil.

MS: m/z 422 (M⁺); IR (NaCl): 3410; 2935; 2818; 1635; 1553; 1516; 1341; 1231; 764; 752. ¹H NMR (CDCl₃, 360 MHz) δ (ppm): 1.60-1.78 (m, 4H, CH₂—CH₂); 2.47 (t, J=7.0 Hz, 2H, CH₂N); 2.60-2.63 (m, 4H, pip); 3.08-3.12 (m, 4H, pip); 3.50-3.55 (m, 2H, CH₂NHCO); 6.84 (ddd, J=1.4 Hz, J=6.9 Hz, J=6.9 Hz, 1H, H-6); 7.00-7.04 (m, 1H, H-arom); 7.05 (d, J=0.9 Hz, 1H, H-3); 7.11-7.16 (m, 2H, H-arom-, H-5); 7.26 (br t, J=5.2 Hz, 1H, NHCO); 7.42-7.48 (m, 1H, H-atom); 7.57-7.60 (m, 1H, H-arom); 7.74 (dd, J=1.6 Hz, 3=7.9 Hz, 1H, H-4); 8.36 (dd, J=0.9 Hz, J=7.0 Hz, 1H, H-7).

### Example 83

### N-4-(4-(4-methoxyphenyl)piperazin-1-yl)butylpyrazolo[1,5-a]pyridin-2-ylcarbamide

Synthesis analogous to example 73.

Yield: 24 mg (47%) white solid matter.

M.P.: 152-154° C.; MS: m/z 407 (M⁺); IR (NaCl): 3356; 2928; 2853; 2816; 1653; 1634; 1550; 1512; 1243; 1033; 756. ¹H NMR (CDCl₃, 360 MHz) δ (ppm): 1.62-1.76 (m, 4H, CH₂—CH₂); 2.47 (t, J=6.8 Hz, 2H, CH₂N); 2.60-2.66 (m, 4H, pip); 3.08-3.16 (m, 4H, pip); 3.49-3.57 (m, 2H, CH₂NHCO); 3.77 (s, 1H, O—CH₃); 6.81-6.85 (m, 3H, H-arom, H-6); 6.86-6.92 (m, 2H, H-arom); 6.94-6.98 (m, 1H, H-arom); 7.05 (d, J=0.9 Hz, 1H, H-3); 7.13 (ddd, J=1.0 Hz, J=6.7 Hz, J=9.0 Hz, 1H, H-5); 7.27 (br t, J=4.8 Hz, H, NHCO); 7.58 (ddd, 3=1.2 Hz, J=1.2 Hz, J=9.0 Hz, 1H, H-4); 8.34 (br dd, J=1.1 Hz, J=7.0 Hz, 1H, H-7). ¹³C NMR (CDCl₃, 90 MHz) δ (ppm): 162.1; 153.8; 148.1; 145.8; 141.3; 128.4; 123.6; 119.3; 118.1; 114.4; 113.5; 97.9; 58.1; 55.6; 53.4; 50.5; 39.1; 27.6; 24.3.

### Example 84

### N-4-(4-(3-chloro-2-methoxyphenyl)piperazin-1-yl)butylpyrazolo[1,5-a]pyridin-2-ylcarbamide

Synthesis analogous to example 73.

Yield: 36 mg (68%) colourless oil.

MS: m/z 440 ((M+2)⁺), 442 ((M+4)⁺). IR (NaCl): 2929; 2853; 2819; 1663; 1635; 1250; 743. ¹H NMR (CDCl₃, 360 MHz) δ (ppm): 1.68-1.71 (m, 4H, CH₂—CH₂); 2.47 (t, J=7.0 Hz, 2H, CH₂N); 2.61-2.64 (m, 4H, pip); 3.14-3.17 (m, 4H, pip); 3.50-3.55 (m, 2H, CH₂NHCO); 3.86 (s, 3H, OCH₃); 6.79 (dd, J=1.8 Hz, J=7.9 Hz, 1H, H-arom); 6.84 (ddd, J=1.2 Hz, J=6.9 Hz, J=6.9 Hz, 1H, H-6); 6.92-6.96 (m, 1H, H-arom); 6.99 (dd, J=1.6 Hz, J=7.9 Hz, 1H, H-arom); 7.05 (d, J=0.9 Hz, 1H, H-3); 7.14 (ddd, J=1.1 Hz, J=6.8 Hz, H=8.9 Hz, 1H, H-5); 7.29 (br t, J=4.8 Hz, 1H, NHCO); 7.58 (br d, J=8.9 Hz, 1H, H-4); 8.35 (br dd, J=1.1 Hz, J=7.0 Hz, 1H, H-7). ¹³C NMR (CDCl₃, 90 Mhz) δ (ppm): 162.4; 148.8; 141.4; 128.4; 124.8; 123.7; 119.3; 117.3; 113.6; 97.8; 59.3; 57.8; 53.4; 49.2; 38.6; 27.4; 21.0.

### Example 85

### N-4-(4-(2,3-dimethylphenyl)piperazin-1-yl)butylpyrazolo[1,5-a]pyridin-2-ylcarbamide

Synthesis analogous to example 73.

Yield: 75 mg (77%) white solid matter.

M.P.: 140-143° C. MS: m/z 405 (M⁺), IR (NaCl): 3410; 2926; 2853; 1658; 1634; 1553; 1241; 1145; 769. ¹H NMR (CDCl₃, 360 MHz) δ (ppm): 1.67-1.74 (m, 4H, CH₂—CH₂); 2.21 (s, 3H, CH₃); 2.26 (s, 3H, CH₃); 2.51 (br t, J=7.0 Hz, 2H, CH₂N); 2.62-2.72 (m, 4H, pip); 2.93-2.94 (m, 4H, pip); 3.52-3.54 (m, 2H, CH₂NHCO); 6.84 (ddd, J=1.2 Hz, J=6.9 Hz, J=6.9 Hz, 1H, H-6); 6.85-7.92 (m, 2H, H-arom); 7.05-7.07 (m, 2H, H-arom, H-3); 7.14 (ddd, J=0.8 Hz, J=6.8 Hz, J=8.7 Hz, 1H, H-5); 7.29 (br, J=4.9 Hz, 1H, NHCO); 7.58 (d, J=9.1 Hz, 1H, H-4); 8.36 (dd, J=30.8 Hz, J=6.8 Hz, 1H, H-7).

### Example 86

### N-4-(4-(2,3-dihydrobenzofuran-7-yl)piperazin-1-yl)butylpyrazolo[1,5-a]pyridin-2-ylcarbamide

Synthesis analogous to example 73, wherein the amine component 4-(4-(2,3-dihydrobenzofuran-7-yl)piperazin-1-yl)butylamine was produced according to the specifications for type C3 amines.

Yield: 72 mg (71%) colourless oil.

M.P.: 60-62° C. MS: m/z 419 (M). IR (NaCl): 3411; 2939; 2817; 1662; 1636; 1553; 1256; 1146; 1012; 753. ¹H NMR (CDCl₃, 360 MHz) δ (ppm): 1.66-1.73 (m, 4H, CH₂—CH₂); 2.54 (t, J=6.8 Hz, 2H, CH₂N); 2.69-2.74 (m, 411, pip); 3.17-3.22 (m, 6H, O—CH₂—CH₂, pip); 3.49-3.55 (m, 2H, CH₂NHCO); 4.59 (t, J=8.9 Hz, 2H, O—CH₂—CH₂); 6.68-6.70 (m, 1H, H-arom); 6.77-6.87 (m, 3H, H-arom, H-6); 7.04 (d, J=0.9 Hz, 1H, H-3); 7.13 (ddd, J=1.0 Hz, J=6.8 Hz, J=8.9 Hz, 1H, H-5); 7.29 (br t, J=5.0 Hz, 1H, NHCO); 7.58 (d, J=8.9 Hz, 1H, H-4); 8.36 (dd, J=0.9 Hz, J=7.0 Hz, 1H, H-7).

### Example 87

### N-4-(chroman-8-yl)piperazin-1-yl)butylpyrazolo[1,5-a]pyridin-2-ylcarbamide

Synthesis analogous to example 73, wherein the amine component 4-(4-(chroman-8-yl)piperazin-1-yl)butylamine was produced according to the specifications for type C3 amines.

Yield: 40 mg (38%) colourless oil.

MS: m/z 434 (M⁺). IR (NaCl); 3397; 2926; 2853; 1634; 1556; 1259; 750. ¹H NMR (CDCl₃, 360 MHz) δ (ppm): 1.70-1.78 (m, 41, CH₂—CH₂); 1.97-2.02 (m, 2H, O—CH₂—CH₂—CH₂); 2.60-2.65 (m, 2H, CH₂N); 2.79 (t, J=6.4 Hz, 2H, O—CH₂—CH₂—CH₂); 2.80-2.87 (m, 4H, pip); 3.14-3.20 (m, 4H, pip); 3.51-3.54 (m, 2H, CH₂NHCO); 4.24-4.26 (m, 2H, O—CH₂—CH₂—CH₂); 6.72-6.78 (m, 3H, H-arom); 6.85 (ddd, J=6.8 Hz, J=6.8 Hz, J=0.1 Hz, 1H, H-6); 7.05 (br s, 1H, H-3); 7.14 (ddd, J=0.8 Hz, J=6.8 Hz, J=8.7 Hz, 1H, H-5); 7.31 (br t, J=5.3 Hz, 1H, NHCO); 7.58 (d, J=9.0 Hz, 1H, H-4); 8.37 (dd, J=0.8 Hz, J=7.2 Hz, 1H, H-7).

### Example 88

### N-4-(4-(Z 4-dimethoxyphenyl)piperazin-1-yl)butylpyrazolo[1,5-a]pyridin-2-ylcarbamide

Synthesis analogous to example 73.

Yield: 74 mg (71%) colourless oil.

MS: m/z 437 (M⁺); IR (NaCl): 3412; 2937; 2817; 1662; 1635; 1552; 1509; 1257; 1208; 1034; 750. ¹H NMR (CDCl₃, 360 MHz) δ (ppm): 1.68-1.74 (m, 4H, CH₂—CH₂); 2.53 (t, J=4.4 Hz, 2H, CH₂N); 2.68-2.76 (m, 4H, pip); 3.04-3.11 (m, 4H, pip); 3.50-3.55 (m, 2H, CH₂NHCO); 3.77 (s, 3H, OCH₃); 3.83 (s, 3H, OCH₃); 6.40-6.43 (m, 1H, H-arom); 6.47-6.48 (m, 1H, H-arom); 6.82-6.87 (m, 2H, H-arom, H-6); 7.05 (d, J=0.7 Hz, 1H, H-3); 7.13 (ddd, J=1.0 Hz, J=6.8 Hz, J=8.9 Hz, 1H, H-5); 7.28 (br t, J=5.2 Hz, 1H, NHCO); 7.58 (d, J=39.0 Hz, 1H, H-4); 8.36 (dd, J=0.9 Hz, J=7.0 Hz, 1H, H-7).

### Example 89

### N-5-(4-(2-methoxyphenyl)piperazin-1-yl)pentyl-4,5,6,7-tetrahydropyrazolo[1,5-a]pyridin-2-ylcarbamide

Synthesis analogous to example 39.

Yield, 40 mg (78%).

MS: 426 m/z (M⁺); ¹H NMR (CDCl₃, 360 MHz) δ (ppm): 1.37-1.46 (m, 2H, CH₂—CH₂—CH₂); 1.52-1.67 (m, 4H, CH₂—CH₂—CH₂); 1.82-1.89 (m, 2H, H-5); 2.01-2.08 (m, 211, H-6); 2.41 (t, J=7.7 Hz, 2H, CH₂N); 2.61-2.68 (m, 4H, pip); 2.81 (t, J=−6.4 Hz, 2H, H-4); 3.06-3.13 (m, 4H, pip); 3.38-3.45 (m, 2H, CH₂NHCO); 3.86 (s, 3H, CH₃O); 4.09-4.16 (m, 2H, H-7); 6.49 (s, 1H, H-3), 6.81-7.01 (m, 5H, H-arom, NHCO).

### Example 90

### N-4-(4-(2-methoxyphenyl)piperazin-1-yl)butyl-6-chloroimidazo[1,2-a]pyridin-2-ylcarbamide

0.024 g 6-chloroimidazo[1,2-a]pyridine-2-carboxylic acid (0.12 mmol) purchased from Ambinter, Paris (F) are dissolved in 4 ml dry methylene chloride and 0.07 ml (0.42 mmol) dry DIPEA are added. Then 0.042 g (0.13 mmol) TBTU dissolved in 0.5 ml dry DMF at 0° C. are slowly droppered in and agitated for 30 minutes at ambient temperature. The reaction solution is again cooled to 0° C. and a solution of 0.034 g (0.13 mmol) 4-(4-(2-methoxyphenyl)piperazin-1-yl)butylamine in 4 ml dry methylene chloride is droppered in. After 1 hour of agitation the reaction deposit is diluted with methylene chloride and washed with saturated NaHCO₃ solution and water. After drying of the organic phase with MgSO₄ the solvent is evaporated and purified by flash chromatography (SiO₂; CH₂Cl₂—CH₃OH:98-2).

Yield: 40 mg (76%) white solid matter.

M.P.: 116-119° C.; MS: m/z 441 (M⁺); IR (NaCl): 3404; 2939; 2819; 1658; 1567; 1499; 1241; 1027; 751. ¹H NMR (CDCl₃, 360 MHz) δ (ppm): 1.61-1.73 (m, 4H, CH₂—CH₂); 2.47 (t, J=7.0 Hz, 2H, CH₂N); 2.63-2.71 (m, 4H, pip); 3.07-3.15 (m, 4H, pip); 3.48-3.53 (m, 2H, CH₂NHCO); 3.86 (s, 3H, OCH₃); 6.84-7.01 (m, 4H, H-arom); 7.20 (dd, J=1.9 Hz, J=9.7 Hz, 1H, H-7); 7.46 (br t, J=5.9 Hz, 1H, NHCO); 7.49 (ddd, J=0.7 Hz, J=0.7 Hz, J=9.7 Hz, 1H, H-8); 8.09 (d, J=0.7 Hz, 1H, H-3); 8.20 (dd, J=0.9 Hz, J=2.0 Hz, 1H, H-5). ¹³C NMR (CDCl₃, 90 MHz) δ (ppm): 162.2; 152.3; 142.8; 141.4; 141.2; 127.5; 124.2; 122.9; 121.7; 121.0; 118.5; 114.3; 111.3; 58.2; 55.4; 53.5; 50.6; 39.1; 27.7; 24.3.

### Example 91

### N-4-(4-(2,3-dichlorophenyl)piperazin-1-yl)butyl-6-chloroimidazo[1,2-a]pyridin-2-yl-carbamide

Synthesis analogous to example 90.

Yield: 77 mg (67%) white solid matter.

M.P.: 135-135° C.; MS: m/z 480 (M⁺); 482 (M⁺+2); IR (NaCl): 3401; 2930; 2820; 1655; 1567; 1449; 1241; 732. ¹H NMR (CDCl₃, 360 MHz) δ (ppm): 1.68-1.75 (m, 4H, CH₂—CH₂); 2.54 (t, J=7.2 Hz, 2H, CH₂N); 2.67-2.76 (m, 4H, pip); 3.09-3.15 (m, 4H, pip); 3.52-3.56 (m, 2H, CH₂NHCO); 6.97 (dd, J=1.9 Hz, J=7.2 Hz, 1H, H-arom); 7.14-7.18 (m, 2H, H-arom); 7.23 (dd, J=1.9 Hz, J=9.4 Hz, 1H, H-7); 7.50 (br t, J=5.7 Hz, 1H, NHCO); 7.51 (d, J=9.8 Hz, 1H, H-8); 8.13 (br s, 1H, H-3); 8.22 (d, J=1.1 Hz, 1H, H-5).

### Example 92

### N-4-4-(2-methoxyphenyl)piperazin-1-yl)butyl-6-chloro-2-methylimidazo[1,2-a]pyridin-3-ylcarbamide

0.025 g 6-chloro-2-methylimidazo[1,2-a]pyridin-3-carboxylic acid (0.12 mmol) purchased from Butt Park Ltd., Camelford (UK) are dissolved in 4 ml dry methylene chloride and 0.07 ml (0.42 mmol) dry DIPEA are added. Then 0.042 g (0.13 mmol) TBTU dissolved in 0.5 ml dry DMF at 0° C. are slowly droppered in and agitated for 15 minutes at ambient temperature. The reaction solution is cooled to 0° C. again and a solution of 0.034 g (0.13 mmol) 4-(4-(2-methoxyphenyl)piperazin-1-yl)butylamine in 4 ml dry methylene chloride is droppered in. After 1 hour the reaction deposit is diluted with methylene chloride and washed with saturated NaHCO₃ solution and water. After drying of the organic phase with MgSO₄ the solvent is evaporated and purified by flash chromatography (SiO₂; CH₂Cl₂—CH₃OH:98-2).

Yield: 45 mg (82%).

M.P.: 118-120° C.; MS: m/z 455 (M⁺); IR (NaCl): 2937; 2818; 1635; 1594; 1498; 1241; 1028; 751. ¹H NMR (CDCl₃, 360 MHz) δ (ppm): 1.66-1.77 (m, 4H, CH₂—CH₂); 2.50 (t, J=6.9 Hz, 2H, CH₂N); 2.65-2.73 (m, 4H, pip); 2.70 (s, 3H, CH₃); 3.04-3.11 (m, 4H, pip); 3.52-3.56 (m, 2H, CH₂NHCO); 3.85 (s, 3H, OCH₃); 6.15 (br t, J=4.9 Hz, 1H, NHCO); 6.84-6.86 (m, 1H, H-arom); 6.88-6.91 (m, 2H, H-arom); 6.95-7.01 (1H, H-arom); 7.28 (dd, J=9.4 Hz, J=1.9 Hz, 1H, H-7); 7.49 (d, J=9.4 Hz, 1H, H-8); 9.45 (d, J=1.1 Hz, 1H, H-5). ¹³C NMR (CDCl₃, 90 MHz) δ (ppm): 161.3; 152.3; 145.5; 144.2; 141.2; 128.1; 126.0; 123.0; 121.4; 121.0; 118.2; 116.7; 116.2; 111.2; 58.1; 55.3; 53.4; 50.4; 39.5; 27.8; 24.3; 16.6.

### Example 93

### N-4-(4-(2-methoxyphenyl)piperazin-1-yl)butylimidazo[1,2-a]pyridin-6-ylcarbamide

0.020 g imidazo[1,2-a]pyridin-6-carboxylic acid (0.12 mmol) are dissolved in 4 ml dry methylene chloride and 0.07 ml (0.42 mmol) dry DIPEA are added. Then 0.042 g (0.13 mmol) TBTU dissolved in 0.5 ml dry DMF at 0° C. are slowly droppered in and agitation takes place for 30 minutes at ambient temperature. The reaction solution is again cooled to 0° C. and a solution of 0.034 g (0.13 mmol) 4-(4-(2-methoxyphenyl)piperazin-1-yl)butylamine in 4 ml dry methylene chloride is droppered in. After 1 hour the reaction deposit is diluted with methylene chloride and washed with saturated NaHCO₃ solution and water. After drying of the organic phase with MgSO₄ the solvent is evaporated and purified by flash chromatography (SiO₂; CH₂Cl₂—CH₃OH:98-2).

Yield: 5 mg (10%) yellow oil.

MS: m/z 408 (M⁺); IR (NaCl); 2933; 2826; 1656; 1553; 1500; 1241; 1027; 750. ¹H NMR (CDCl₃, 360 MHz) δ (ppm): 1.68-1.76 (m, 4H, CH₂—CH₂); 2.52 (t, J=6.6 Hz, 2H, CH₂N); 2.67-2.74 (m, 4H, pip); 3.05-3.15 (m, 4H, pip); 3.49-3.52 (m, 2H, CH₂NHCO); 3.85 (s, 3H, OCH₃); 6.84-6.93 (m, 3H, H-arom); 6.97-7.02 (m, 1H, H-arom); 7.04 (br t, J=6.4 Hz, 1H, NHCO); 7.44 (dd, J=31.5 Hz, J=9.4 Hz, in, H-3); 7.61 (d, J=9.4 Hz, 1H, H-2); 7.65 (br s, 1H, H-7); 7.69 (d, J=1.1 Hz, 1H, H-8); 8.82 (br s, 1H, H-5).

### Example 94

### N-4-(4-(2,3-dichlorophenyl)piperazin-1-yl)butyl[1,2,4]triazolo[1,5-a]pyridin-2-ylcarbamide

0.01 g [1,2,4]triazolo[1,5-a]pyridine-2-carboxylic acid (0.06 mmol) are dissolved in 3 ml dry methylene chloride and 0.035 ml (0.12 mmol) dry DIPEA are added. Then 0.022 g (0-07 mmol) TBTU dissolved in 0.5 ml dry DMF at 0° C. are slowly droppered in and agitation takes place for 30 minutes at ambient temperature. The reaction solution is cooled to 0° C. again and a solution of 0.03 g (0.1 mmol) 4-(4-(2,3-dichlorophenyl)piperazin-1-yl)butylamine in 4 ml dry methylene chloride is droppered in at 0° C. After 1 hour the reaction deposit is diluted with methylene chloride and washed with saturated NaHCO₃ solution and water. After drying of the organic phase with MgSO₄ the solvent is evaporated and purified by flash chromatography (SiO₂; CH₂Cl₂—CH₃OH:97-3).

Yield: 20 mg (74%) white solid matter.

M.P.: 134-135° C.; MS; m/z 446 ((M+2)⁺), 448 ((M+4)⁺). IR (NaCl); 2941; 2820; 1676; 1637; 1241; 734.11H NMR (CDCl₃, 360 MHz) δ (ppm): 1.67-1.77 (m, 4H, CH₂—CH₂); 2.55 (t, J=7.0 Hz, 2H, CH₂N); 2.69-2.75 (m, 4H, pip); 3.10-3.14 (m, 4H, pip); 3.56-3.61 (m, 2H, CH:NHCO); 6.96-6.99 (m, 1H, H-6); 7.10-7.17 (m, 3H, H-arom); 7.60-7-66 (m, 2H, H-7, NHCO); 7.77-7.80 (m, 1H, 14-8); 8.66-8.69 (m, 1H, H-5).

### Example 95

### N-4-(4-(2,3-dichlorophenyl)piperazin-1-yl)pyrazolo[1,5-b]pyridazin-2-ylcarbamide

0.019 g pyrazolo[1,5-b]pyridazin-2-carboxylic acid (0.12 mmol) are dissolved in 5 ml dry methylene chloride and 0.07 ml (0.42 mmol) dry DIPEA are added. Then 0.042 g (0.13 mmol) TBTU dissolved in 0.5 ml dry DMF at 0° C. are slowly droppered in and agitation takes place for 30 minutes at ambient temperature. The reaction solution is cooled to 0° C. again and a solution of 0.085 g (0.28 mmol) 4-(4-(2,3-dichlorophenyl)piperazin-1-yl)butylamine in 4 ml dry methylene chloride is droppered in at 0° C. After 1 hour the reaction deposit is diluted with methylene chloride and washed with saturated NaHCO₃ solution and water. After drying of the organic phase with MgSO₄ the solvent is evaporated and purified by flash chromatography (SiO₂; CH₂Cl₂—CH₃OH:97-3).

Yield: 39 mg (72%) white solid matter.

M.P.: 122-124° C.; MS: m/z 446 ((M+2)⁺), 448 ((M+4)⁺). IR NaCl): 2934; 2821; 1656; 1242; 725. ¹H NMR (COC₃, 360 MHz) δ (ppm). 1.69-1.74 (m, 4H, CH₂—CH₂); 2.59 (t, J=6.9 Hz, 2H, CH₂N); 2.74-2.80 (m, 4H, pip); 3.11-3.16 (m, 4H, pip); 3.51-3.57 (m, 2H, CH₂NHCO); 6.98 (dd, J=2.5 Hz, J=7.0 Hz, 1H, H-arom); 7.05 (dd, J=4.5 Hz, J=9.1 Hz, 1H, H-5); 7.13-7.16 (m, 2H, H-arom); 7.19 (s, 1H, H-3); 7.47 (br t, J=5.4 Hz, 1H, NHCO); 8.03 (dd, J=2.0 Hz, J=9.1 Hz, 1H, H-4); 8.36 (dd, J=1.9 Hz, J=4.4 Hz, 1H, H-6).

### Example 96

### N-4-(4-(2-methoxyphenyl)piperazin-1-yl)butyl-6-chloroimidazo[1,2-b]pyridazin-2-ylcarbamide

0.047 g 6-chloroimidazo[1,2-b]pyridazin-2-carboxylic acid (0.24 mmol) purchased from Maybridge, Tintagel (UK) are dissolved in 6 ml dry methylene chloride and 0.14 ml (0.84 mmol) dry DIPEA are added. Then 0.084 g (0.26 mmol) TBTU dissolved in 0.5 ml dry DMF at 0° C. are slowly droppered in and agitation takes place for 15 Minutes at ambient temperature. The reaction solution is cooled to 0° C. and a solution of 0.071 g (0.27 mmol) 4-(4-(2-methoxyphenyl)piperazin-1-yl)butylamine in 4 ml dry methylene chloride is droppered in. After 1 hour the reaction deposit is diluted with methylene chloride and washed with saturated NaHCO₃ solution and water. After drying of the organic phase with MgSO₄ the solvent is evaporated and purified by flash chromatography (SiO₂; CH₂Cl₂—CH₃OH:98-2).

Yield: 82 mg (77%).

M.P.: 120-123° C.; MS: m/z 442 (M⁺); IR (NaCl): 3407; 2931; 2851; 2817; 1655; 1499; 1241; 1028; 751. ¹H NMR (CDCl₃, 360 MHz) δ (ppm): 1.64-1.74 (m, 4H, CH₂—CH₂); 2.50 (t, J=7.0 Hz, 2H, CH₂N); 2.65-2.73 (m, 4H, pip); 3.09-3.17 (m, 4H, pip); 3.49-3.55 (m, 2H, CH₂(NHCO); 3.86 (s, 3H, OCH₃); 6.84-7.02 (m, 4H, H-arom); 7.11 (d, J=9.5 Hz, 1H, H-8); 7.48 (br t, J=5.3 Hz, 1H, NHCO); 7.84 (d, J=9.5 Hz, 1H, 14-7); 8.43 (d, J=0.7 Hz, 1H, H-3).

### Example 97

### N-4-(4-(2-methoxyphenyl)piperazin-1-yl)butyl-6-chloro-2-phenylimidazo[1,2-b]pyridazin-3-ylcarbamide

0.033 g 6-chloro-2-phenylimidazo[1,2-b]pyridazin-3-carboxylic acid (0.12 mmol) purchased from Butt Park Ltd., Camelford (UK) are dissolved in 4 ml dry methylene chloride and 0.07 ml (0.42 mmol) dry DIPEA are added. Then 0.042 g (0.13 mmol) TBTU dissolved in 0.5 ml dry DMF at 0° C. are slowly droppered in and agitation takes place for 30 minutes at ambient temperature. The reaction solution is cooled to 0° C. again and a solution of 0.034 g (0.13 mmol) 4-(4-(2-methoxyphenyl)piperazin-1-yl)butylamine in 4 ml dry methylene chloride is droppered in. After 1 hour the reaction deposit is diluted with methylene chloride and washed with saturated NaHCO₃ solution and water. After drying of the organic phase with MgSO₄ the solvent is evaporated and purified by flash chromatography (SiO₂; CH₂Cl₂—CH₃OH:98-2).

Yield: 41 mg (66%) white solid matter.

M.P., 58-62° C.; MS: m/z 518 (M⁺), 520 (M+2)⁺; IR (NaCl): 3348; 2929; 2816; 1656; 1554; 1499; 1241; 1027; 751. ¹H NMR (CDCl₃, 360 MHz) δ ppm): 1.66-1.74 (m, 4, CH₂—CH₂); 2.41-2.50 (m, 2H, CH₂N); 2.58-2.66 (m, 4H, pip); 2.98-3.09 (m, 4H, pip); 3.53-3.60 (m, 2H, CH₂NHCO); 3.85 (s, 3H, OCH₃); 6.82-6.93 (m, 4H, H-arom); 6.96-7.01 (m, 1H, H-arom); 7.21 (d, J=9.4 Hz, 1H, H-8); 7.35-7.48 (m, 3H, H-arom); 7.89-7.95 (m, 2H, H-arom); 8.01 (d, J=9.4 Hz, 1H, H-7); 8.51 (br t, J=4.8 Hz, 1H, NHCO); 8.58 (br t, J=5.3 Hz, 1H, NH).

Creation of Substance Libraries by Solid-Phase-Supported Synthesis:

The creation of a substance library of dopamine receptor-affine ligands by solid-phase-supported synthesis is based on methods which have been developed by our working group and previously described (Löber, S., et al. Org. Lett. 2003, 5, 1753-1755). The basis for this work is the development of novel HAL linkers, which led to the synthesis of the FIMT (formyl-indolyl-methyl-triazole) resin. With the help of this functionalised polystyrene it was possible to synthesise two libraries (Library I and Library 2) of potent ligands;

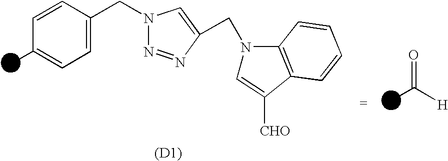

In the following the individual reaction steps of the solid-phase-supported synthesis of N-(ω-(4-(2,3-dichlorophenyl)piperazin-1-yl)alkylheteroarylcarbamides (Library 1 with examples 6-9, 18-21, 24-27, 31-38, 41-44) are described.

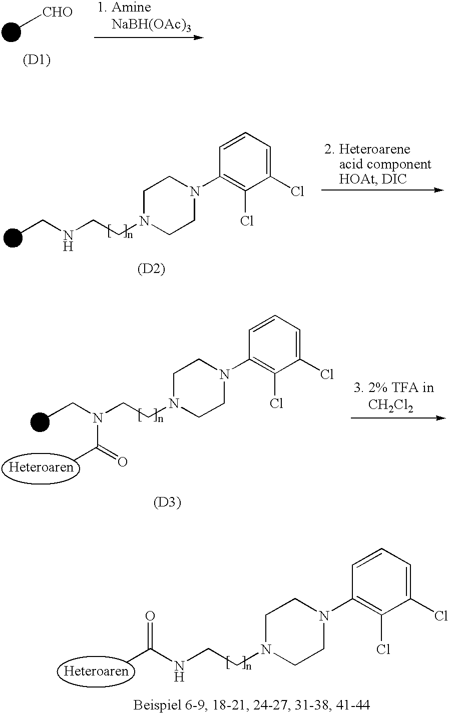

In Library 2 the compounds of example 13 and examples 50-68 were produced analogously to this synthesis sequence.

Production of the Polymer-Combined Compound D2 (Step 1):

### Polymer-combined ω-(4-(2,3-dichlorophenyl)piperazin-1-yl)alkylamine

0.100 g (1.043 mmol/g) FIMT resin (D1), 4 eq. NaBH(AcO)₃ and a solution of 4 eq. of the amine component in 5 ml dry methylene chloride are agitated for 24 hours at ambient temperature in a Teflon reaction vessel (PLS Organic Synthesiser; rotation: 320/min). Then the resin is filtered off and goes through 3 subsequent washing stages: methanol, methanol-0.1 N HCl (9-1), triethylamine (2% in methylene chloride) and methylene chloride. After the final washing process the resin is dried in the course of its filtration.

Production of the Polymer-Combined Compound D3 (Step 2):

### Polymer-combined N-(ω-(4-(2,3-dichlorophenyl)piperazin-1-yl)alkyl)heteroarene carbamide

The resin obtained is suspended in 5 ml methylene chloride-DMF:9-1 and 4 Eq acid, 4 eq. HOAt and 4.5 eq. N,N′-diisopropylcarbodiimide are added. The reaction deposit is agitated for 48 hours at ambient temperature, and then it is filtered off and washed 3 times with DMF, methanol and dichloromethane and after the final washing stage is dried in the frit.

Separation of the Polymer-Combined Target Compounds Examples 6-9, 18-21, 24-27, 31-38, 41-44 and 13 and 50-68 (Step 3):

### N-(ω-(4-(2,3-dichlorophenyl)piperazin-1-yl)alkyl)heteroarene carbamide

5 ml of a solution of 2% trifluoroethanoic acid in methylene chloride are added to the resin obtained and agitation takes place for 2 hours at ambient temperature. The resin is filtered, and washed twice each with 3 ml methanol and then methylene chloride. The filtrate and washing fluids are combined, the solvent evaporated and in doing so the product obtained.

Characterisation of the Solid-Synthesised Substances:

The analytical check of the products obtained by solid-phase-supported synthesis is performed by LC/MS analysis on a chromatography system from the company Agilent (Binary Gradient System in combination with the ChemStation Software) and the mass spectrometry determination with the help of a Bruker Esquire 2000 ion-trap mass spectrometer (ionisation in an APCI source).

The chromatographic separation was performed on a Zorbax SB-C18 column (4-6 mm ID×250 mm, 5 μm) with an MeOH/0.1 N aq.HCO₂H solvent system in gradients from 50/50 to 90/10 at a flow rate of 0.5 ml/min. Detection was performed by means of Agilent UV/VIS-detector at 254 nm.

Compounds of Substance Libraries 1 and 2:

All compounds of substance libraries 1 and 2 were analytically characterised as discussed above and investigated in a biological screening procedure. The most promising test compounds then underwent a detailed spectroscopic investigation and were characterised by further receptor bonding experiments.

Compounds of Library 1:

### Example 6

### N-2-(4-(2,3-dichlorophenyl)piperazin-1-yl)ethylpyrazolo[1,5-a]pyridin-2-ylcarbamide

Molecular weight (MW) (calculated): 418.33; Mass (MS) (actual): 418.7 (M+1); Retention time (T_{R} in [min]): 11.2.

### Example 7

### N-3-(4-(2,3-dichlorophenyl)piperazin-1-yl)propylpyrazolo[1,5-a]pyridin-2-ylcarbamide

MW (calculated): 432.36; MS (actual): 432.4; T_{R}: 12.0 min.

### Example 8

### N-4-(4-(2,3-dichlorophenyl)piperazin-1-yl)butylpyrazolo[1,5-a]pyridin-2-ylcarbamide trifluoroethanoic acid salt

MW (calculated): 446.68; MS (actual): 446.5; T_{R}: 6.1 min.

IR (NaCl): 3410, 3318, 2954, 2849, 1778, 1670, 1635, 1555, 1514, 1452, 1198. ¹H NMR (CDCl₃, 360 MHz) δ (ppm): 1.74-1.80 (m, 2H, CH₂—CH₂—Npip); 1.89-1.95 (m, 2H, CONH—CH₂CH₂); 3.06-3.12 (m, 2H, CH₂Npip); 3.20-3.41 (m, 6H, pip); 3.52-3.58 (m, 2H, CH₂NHCO); 3.69-3.72 (m, 2H, pip); 5.30 (br s, 1H, HNpip); 6.86-6.89 (m, 1H, phenyl); 6-97-7.00 (m, 1H, H-6), 7.05 (s, 1H, H-3); 7.14-7.27 (m, 3H, phenyl, H-5), 7.42 (s, 1H, HNCO), 7.60 (d, J=8.2 Hz, 1H, H-4), 8.49 (d, J=6.7 Hz, 1H, H-7).

### Example 9

### N-5-(4-(2,3-dichlorophenyl)piperazin-1-yl)pentylpyrazolo[1,5-a]pyridin-2-ylcarbamide trifluoroethanoic acid salt

MW (calculated): 460.41; MS (actual): 460.0; T_{R}: 9.1 min.

IR (NaCl): 3407, 3326, 2948, 1773, 1671, 1558, 1514, 1451, 1199. ¹H NMR (CDCl₃, 360 MHz) δ (ppm): 1.46-1.54 (m, 2H, CH₂CH₂CH₂); 1.67-1.75 (m, 2H, CH₂—CH₂—Npip); 1.84-1.93 (m, 2H, CH₂Npip); 3.04-3.13 (m, 4H, CONH—CH₂CH₂, pip); 3.28-3.42 (m, 4H, pip); 3.49-3.54 (m, 2H, CH₂NHCO); 3.70-3.74 (m, 2H, pip); 6.24 (br s, 1H, HNpip); 6.86-6.89 (m, 1H, phenyl); 6-97-7.00 (m, 1H, H-6), 7.06 (s, 1H, H-3); 7.13-7.26 (m, 3H, phenyl, H-5), 7.30 (s, 1H, HNCO), 7.60 (d, J=7.8 Hz, 1H, H-4), 8.39 (d, J=6.4 Hz, 1H, H-7).

### Example 18

### N-2-(4-(2,3-dichlorophenyl)piperazin-1-yl)ethylpyrazolo[1,5-a]pyridin-3-ylcarbamide

MW (calculated): 418.33; MS (actual): 418.7; T_{R}: 11.0 min.

### Example 19

### N-3-(4-(2,3-dichlorophenyl)piperazin-1-yl)propylpyrazolo[1,5-a]pyridin-3-ylcarbamide

MW (calculated): 432.36; MS (actual): 432.2; T_{R}: 8.4 min.

### Example 20

### N-4-(4-(2,3-dichlorophenyl)piperazin-1-yl)butylpyrazolo[1,5-a]pyridin-3-ylcarbamide

MW (calculated): 446.68; MS (actual): 446.1; T_{R}: 6.5 min.

### Example 21

### N-5-(4-(2,3-dichlorophenyl)piperazin-1-yl)pentylpyrazolo[1,5-a]pyridin-3-ylcarbamide

MW (calculated): 460.41; MS (actual): 460.1; T_{R}: 14.3 min.

### Example 24

### N-2-(4-(2,3-dichlorophenyl)piperazin-1-yl)ethylpyrazolo[1,5-a]pyridin-5-ylarbamide

MW (calculated): 418.33; MS (actual): 418.5; T_{R}: 1.0 min.

### Example 25

### N-3-(4-(2,3-dichlorophenyl)piperazin-1-yl)propylpyrazolo[1,5-a]pyridin-5-ylcarbamide

MW (calculated): 432.36; MS (actual): 431.9; T_{R}: 9.9 min.

### Example 26

### N-4-(4-(2,3-dichlorophenyl)piperazin-1-yl)butylpyrazolo[1,5-a]pyridin-5-ylcarbamide trifluoroethanoic acid salt

MW (calculated): 446.68; MS (actual): 446.0; T_{R}: 9.8 min.

IR (NaCl): 3325, 2961, 2851, 1779, 1671, 1631, 1548, 1452, 1201. ¹H NMR (CDCl₃, 360 MHz) δ (ppm): 1.74-1.85 (m, 2H, CH₂CH₂Npip); 1.95-2.06 (m, 2H, CONHCH₂CH₂); 3.03-3.12 (m, 2H, CH₂Npip); 3.14-3.23 (m, 2H, pip); 3.28-3.44 (m, 4H, pip); 3.51-3.70 (m, 4H, CH₂NHCO, pip); 6.70 (br s, 1H, HNpip); 6.97-6.99 (m, 1H, H-3); 6.99-7.01 (m, 1H, phenyl); 7.17-7.23 (m, 21, phenyl); 7.24-7.26 (m, 1H, H-6), 7.65 (br s, IRH, HNCO); 8.01 (s, 1H, H-4), 8.14-8.27 (t, 1H, H-2), 8.45-8.57 (m, 1H, Hz, H-7).

### Example 27

### N-5-(4-(2,3-dichlorophenyl)piperazin-1-yl)pentylpyrazolo[1,5-a]pyridin-5-ylcarbamide trifluoroethanoic acid salt

MW (calculated): 460.41; MS (actual): 460.0; T_{R}: 11.2 min.

IR (NaCl): 3326, 2948, 2861, 1778, 1672, 1631, 1548, 1452, 1200. ¹H NMR (CDCl₃, 360 MHz) δ (ppm): 1.48-1.59 (m, 2H, CH₂CH₂CH₂); 1.66-1.77 (m, 2H, CH₂CH₂Npip); 1.85-1.97 (m, 2H, CONHCH₂CH₂); 3.02-3.19 (m, 4H, CH₂Npip, pip); 3.26-3.35 (m, 2H, pip); 3.36-3.46 (m, 3H, pip); 3.47-3.58 (m, 1H, pip); 3.64-3.75 (m, 2H, CH₂NHCO); 6.73 (br s, 1H, HNpip); 6.96-6.98 (m, 1H, H-3); 6.98-7.00 (m, 1H, phenyl); 7.17-7.24 (m, 4H, phenyl, U-6, HNCO); 8.00-8.80 (m, 3H, H-4, H-2, H-7).

### Example 31

### N-2-(4-(2,3-dichlorophenyl)piperazin-1-yl)ethylpyrazolo[1,5-a]pyridin-6-ylcarbamide

MW (calculated). 418.33; MS (actual): 417.9; T_{R}: 7.3 min.

### Example 32

### N-3-(4-(2,3-dichlorophenyl)piperazin-1-yl)propylpyrazolo[1,5-a]pyridin-6-ylcarbamide

MW (calculated): 436.39; MS (actual): 431.9; T_{R}: 8.6 min.

### Example 33

### N-4-(4-(2,3-dichlorophenyl)piperazin-1-yl)butylpyrazolo[1,5-a]pyridin-6-ylcarbamide

MW (calculated): 446.68; MS (actual): 446.3; T_{R}: 7.4 min.

### Example 34

### N-5-(4-(2,3-dichlorophenyl)piperazin-1-yl)pentylpyrazolo[1,5-a]pyridin-6-ylcarbamide

MW (calculated): 460.41; MS (actual): 460.0; T_{R}: 6.0 min.

### Example 35

### N-2-(4-(2,3-dichlorophenyl)piperazin-1-yl)ethyl-4,5,6,7-tetrahydropyrazolo[1,5-a]pyridin-2-ylcarbamide

MW (calculated): 422.36; MS (actual): 422.4; T_{R}: 7.2 min.

### Example 36

### N-3-(4-(2,3-dichlorophenyl)piperazin-1-yl)propyl-4,5,6,7-tetrahydropyrazolo[1,5-a]pyridin-2-ylcarbamide

MW (calculated): 436.39; MS (actual): 436.4; T_{R}: 6.3 min.

### Example 37

### N-4-(4-(2,3-dichlorophenyl)piperazin-1-yl)butyl-4,5,6,7-tetrahydropyrazolo[1,5-a]pyridin-2-ylcarbamide trifluoroethanoic acid salt

MW (calculated): 450.42; MS (actual): 450.5; T_{R}: 6.2 min.

IR NaCl): 3407, 3328, 2956, 2867, 1776, 1669, 1631, 1578, 1534, 1451, 1197. ¹H NMR (CDCl₃, 360 MHz) δ (ppm)) 1.69-1.73 (m, 2H, CH₂CH₂Npip); 1.84-1.89 (m, 4H, CONH—CH₂CH₂, H-5); 2.02-2.08 (m, 2H, H-6); 2.80-2.84 (m, 2H, H-4); 3.07-3.12 (m, 2H, CH₂Npip); 3.19-3.31 (m, 4H, pip); 3.38-3.48 (m, 4H, pip); 3.68-3.72 (m, 2H, CH₂NHCO); 4.11-4.15 (m, 2H, H-7); 6.50 (s, 1H, H-3); 6.96-6.98 (m, 1H, phenyl); 7.16-7.26 (m, 3H, phenyl, HNCO), 8.27 (br s, 1H, HNpip).

### Example 38

### N-5-(4-(2,3-dichlorophenyl)piperazin-1-yl)pentyl-4,5,6,7-tetrahydropyrazolo[1,5-a]pyridin-2-ylcarbamide trifluoroethanoic acid salt

MW (calculated). 464.44; MS (actual). 464.1; T_{R}: 6.4 min.

IR (NaCl): 3412, 3327, 2951, 2865, 1779, 1669, 1631, 1578, 1533, 1452, 1198. ¹H NMR (CDCl₃, 360 MHz) δ (ppm): 1.43-1.49 (m, 2H, CH₂CH₂CH₂); 1.63-1.67 (m, 2H, CH₂CH₂Npip); 1.81-1.90 (m, 4H, CONHCH₂CH₂, H-5); 2.03-2.05 (m, 2H, H-6); 2.80-2.83 (m, 2H, H-4); 3.03-3.12 (m, 4H, pip); 3.30 (t, J=11.7 Hz, 2H, CH₂Npip); 3.38-3.45 (m, 4H, pip); 3.69-3.73 (m, 2H, CH₂NHCO); 4.11-4.14 (m, 2H, H-7); 6.50 (s, 1H, H-3); 6.98-7.00 (m, 1H, phenyl); 7.08 (br s, 1H, HNCO); 7.17-7.27 (m, 21, phenyl), 7.83 (br s, 1H, HNpip).

### Example 41

### N-2-(4-(2,3-dichlorophenyl)piperazin-1-yl)ethyl-4,5,6,7-tetrahydropyrazolo[1,5-a]pyridin-3-ylcarbamide

MW (calculated): 422.36; MS (actual): 421.9; T_{R}: 6.6 min.

### Example 42

### N-3-(4-(2,3-dichlorophenyl)piperazin-1-yl)propyl-4,5,6,7-tetrahydropyrazolo[1,5-a]pyridin-3-ylcarbamide

MW (calculated): 450.42; MS (actual): 436.4; T_{R}: 5.9 min.

### Example 43

### N-4-(4-(2,3-dichlorophenyl)piperazin-1-yl)butyl-4,5,6,7-tetrahydropyrazolo[1,5-a]pyridin-3-ylcarbamide

MW (calculated): 450.42; MS (actual): 450.5; T_{R}: 6.2 min.

### Example 44

### N-5-(4-(2,3-dichlorophenyl)piperazin-1-yl)pentyl-4,5,6,7-tetrahydropyrazolo[1,5-a]pyridin-3-ylcarbamide

MW (calculated): 464.44; MS (actual): 464.1; T_{R}: 10.8 min.

Compounds of Library 2:

### Example 13

### N-4-(4-(2-methoxyphenyl)piperazin-1-yl)butyl-6-bromopyrazolo[1,5-a]pyridin-2-ylcarbamide

MW (calculated): 486.42; MS (actual): 487.7; T_{R}: 19.3 min.

The spectroscopic data from example 13 is also described on page 63.

### Example 50

### N-4-(4-(2,3-dichlorophenyl)piperazin-1-yl)butyl-5-methoxypyrazolo[1,5-a]pyridin-2-ylcarbamide

MW (calculated): 476.41; MS (actual): 476.6; T_{R}: 17.2 min.

### Example 51

### N-4-(4-(2-methoxyphenyl)piperazin-1-yl)butyl-5-methoxypyrazolo[1,5-a]pyridin-2-ylcarbamide

MW (calculated): 437.55; MS (actual): 438.2; T_{R}: 18.5 min.

### Example 52

### N-4-(4-(2,3-dichlorophenyl)piperazin-1-yl)butyl-5-trifluoromethylpyrazolo[1,5-a]pyridin-2-ylcarbamide

MW (calculated): 514.38; MS (actual): 514.8; T_{R}: 18.2 min.

### Example 53

### N-4-(4-(2-methoxyphenyl)piperazin-1-yl)butyl-5-triflutoromethylpyrazolo[1,5-a]pyridin-2-ylcarbamide

MW (calculated): 475.52; MS (actual): 476.2; T_{R}: 19.7 min.

### Example 54

### N-4-(4-(2,3-dichlorophenyl)piperazin-1-yl)butyl-6-bromopyrazolo[1,5-a]pyridin-2-ylcarbamide

MW (calculated). 525.28; MS (actual): 526.0; T_{R}: 17.9 min.

### Example 55

### N-4-(4-(2,3-dichlorophenyl)piperazin-1-yl)butyl-6-chloropyrazolo[1,5-a]pyridin-2-ylcarbamide

MW (calculated): 480.82; MS (actual): 481.8; T_{R}: 17.9 min.

### Example 56

### N-4-(4-(2-methoxyphenyl)piperazin-1-yl)butyl-6-chloropyrazolo[1,5-a]pyridin-2-ylcarbamide

MW (calculated): 441.96; MS (actual): 442.4; T_{R}: 19.1 min.

### Example 57

### N-4-(4-(2,3-dichlorophenyl)piperazin-1-yl)butyl-6-fluoropyrazolo[1,5-a]pyridin-2-ylcarbamide

MW (calculated): 464.37; MS (actual): 464.5; T_{R}: 17.4 min.

### Example 58

### N-4-(4-(2-methoxyphenyl)piperazin-1-yl)butyl-6-fluoropyrazolo[1,5-a]pyridin-2-ylcarbamide

MW (calculated): 425.51; MS (actual): 426.2; T_{R}: 18.4 min.

### Example 59

### N-5-(4-(2,3-dichlorophenyl)piperazin-1-yl)pentyl-5-methoxypyrazolo[1,5-a]pyridin-2-ylcarbamide

MW (calculated): 490.43; MS (actual): 490.7; T_{R}: 17.5 min.

### Example 60

### N-5-(4-(2-methoxyphenyl)piperazin-1-yl)pentyl-5-methoxypyrazolo[1,5-a]pyridin-2-ylcarbamide

MW (calculated): 451.57; MS (actual): 452.4; T_{R}: 18.7 min.

### Example 61

### N-5-(4-(2,3-dichlorophenyl)piperazin-1-yl)pentyl-5-trifluoromethylpyrazolo[1,5-a]pyridin-2-ylcarbamide

MW (calculated): 528.4; MS (actual): 529.5; T_{R}: 18.1 min.

### Example 62

### N-5-(4-(2-methoxyphenyl)piperazin-1-yl)pentyl-5-trifluoromethylpyrazolo[1,5-a]pyridin-2-ylcarbamide

MW (calculated): 484.54; MS (actual): 490.2; T_{R}: 19.8 min.

### Example 63

### N-5-(4-(2,3-dichlorophenyl)piperazin-1-yl)pentyl-6-bromopyrazolo[1,5-a]pyridin-2-ylcarbamide

MW (calculated): 539.30; MS (actual): 540.0; T_{R}: 18.0 min.

### Example 64

### N-5-(4-(2-methoxyphenyl)piperazin-1-yl)pentyl-6-bromopyrazolo[1,5-a]pyridin-2-ylcarbamide

MW (calculated): 500.44; MS (actual): 501.7; T_{R}: 19.4 min.

### Example 65

### N-5-(4-(2,3-dichlorophenyl)piperazin-1-yl)pentyl-6-chloropyrazolo[1,5-a]pyridin-2-ylcarbamide

MW (calculated): 494.84; MS (actual): 495.8; T_{R}: 17.9 min.

### Example 66

### N-5-(4-(2-methoxyphenyl)piperazin-1-yl)pentyl-6-chloropyrazolo[1,5-a]pyridin-2-ylcarbamide

MW (calculated): 455.98; MS (actual): 456.4; T_{R}: 19.2 min.

### Example 67

### N-5 (4-(2,3-dichlorophenyl)piperazin-1-yl)pentyl-6-fluoropyrazolo[1,5-a]pyridin-2-ylcarbamide

MW (calculated): 478.34; MS (actual): 478.7; T_{R}: 17.4 min.

### Example 68

### N-5-(4-(2-methoxyphenyl)piperazin-1-yl)pentyl-6-fluoropyrazolo[1,5-a]pyridin-2-ylcarbamide

MW (calculated): 439.53; MS (actual). 440.2; T_{R} 18.6 min.

Biological Activity

The biological activities of the compounds according to the invention were determined in radioligand bonding experiments. All radioligand experiments were performed according to methods described by us (Hübner, H. et al. J. Med. Chem. 2000, 43, 756-762). For the measurement of the affinities to the receptors of the D2-family membrane homogenates of Chinese hamster ovary cells (CHO cells) were used, which stably express the human D2long-, the human D2short—(Hayes, G. et al. Mol. Endocrinol. 1992, 6, 920-926), the human D3—(Sokoloff, P. et al. Eur. J. Pharmacol. 1992, 225, 331-337) or the human D4.4-receptor sub-type, (Asghari, V. J. Neurochem. 1995, 65, 1157-1165) respectively. Basically the binding assays took place by incubation of the receptor homogenates with the radioligand [³H]spiperone and the compound under investigation in various concentrations. Determination of the affinities to the D1 receptor took place with native membrane homogenates, obtained from porcine striatum, and the D1-selective radioligands [3H]SCH 23390.

Biological testing of the substances obtained by solid-phase-supported synthesis took place initially according to a screening approach. Here the test bonds were investigated for their capacity, at representative concentrations of the radioligands, to displace the radioligands from the binding position of the respective receptor subtype. For the most promising candidates in subsequent dose-effect-investigations the binding characteristics to the various dopamine receptor subtypes were determined and examples of the affinities to the 5-HT1A-, 5-HT2- and α1-receptors ascertained.

Measurement of the bonding strengths of the compounds to the serotonin-receptor subtypes 5-HT1A and 5-HT2 was carried out according to methods described by us (Heindl, C. et al. Tetrahedron: Asymmetry 2003, 14, 3141-3152). For this we incubated porcine cortex-membrane preparations with the radioligands [³H]8-OH-DPAT (for 5-HT1A) or [³H]ketanserin (5-HT2) and the compounds in various concentrations. In the same way the affinity of the test compounds to the porcine α1-receptor was investigated, wherein porcine cortex-membrane preparations and the α1-selective radioligand [³H]prazosin were used.

All compounds investigated in the dopamine receptor-binding assay demonstrated good to very good affinities to the dopamine receptors with a clear binding preference to subtypes of the 1)₂ family. Independently of the partial structure, there is always a clear selectivity to the D3 receptor here, which for all the compounds tested was bonded with Ki-values of between 0.1 and approximately 200 nM.

Investigations to determine the intrinsic activity of the example compounds were carried out in a mitogenesis assay in accordance with the literature (Hübner, H. et al. J. Med. Chem. 2000, 43, 4563-4569; Bettinetti, L. et al. J. Med. Chem. 2002, 45, 4594-4597). Here various concentrations of the compounds under investigation were incubated with 1)₃ receptor-expressing cells and then the receptor-mediated stimulation of the mitogenesis rate was measured by incorporation of the radioactive marker [³H]thymidine. Agonistic, partial agonistic or antagonistic effects were determined in comparison with the effect of the full agonist quinpirol.

In this test the compounds under investigation demonstrate differing intrinsic effects at the D3-receptor. So some example have no [³H]thymidine incorporation and can thus be classified as antagonists. Other compounds demonstrate a stimulation of the receptor in the range 11%-35% and can rather be classified as weakly partially agonistic, whereas a third group of substances with an intrinsic activity of 36-50% can be classified as partial agonists.

1. A compound of general formula I 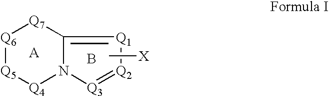
in which wherein:
A is a saturated or aromatic 6-membered ring;
B is an aromatic 5-membered ring;
the heteroarene formed by A+B has in total a maximum of three ring-forming N-atom and precisely one X group as substituents;
Q₁, Q₂ and Q₃ are in each case and independently of each other N, CH or C—R1;
Q₄ is N—R, CH—R1′ or C—R1R1′;
Q5, Q6 and Q7 are independently of each other CH—R1′ or C—R1R1′;
R1 is independently in each case hydroxy, alkyl, alkyloxy, alkylthio, alkenyl, alkynyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl or alkylsulfonylamino;
R1′ is absent if ring A is aromatic, or is hydrogen if ring A is saturated;
R is absent if ring A is aromatic, or is hydrogen, alkyl, phenyl, alkylcarbonyl, phenylcarbonyl, phenylalkyl or phenylsulfonyl, if ring A is saturated;
X is a group of general formula X1 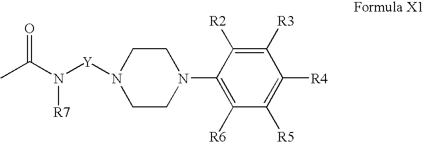
bonded to a C-atom of an aromatic ring A or B, wherein:
Y is an unbranched, saturated or unsaturated hydrocarbon chain with 2-5 carbon atoms or a chain —(CH₂)ₒ-Z-(CH₂)ₚ, wherein Z is a cyclopentyl, cyclohexyl or cycloheptyl residue, wherein o and p in each case and independently of each other have the value 0, 1, 2 or 3, and wherein the sum of o and p is a maximum of 3;
R2, R3, R4, R5 and R6 are in each case and independently of each other hydrogen, hydroxy, alkyl, alkyloxy, alkylthio, alkenyl, alkynyl, phenyl, phenylalkyl, phenoxy, phenylalkyloxy, halogen, trifluoromethyl, alkylcarbonyl, phenylcarbonyl, alkyloxycarbonyl, phenylalkyloxycarbonyl, cyano, nitro, amino, carboxy, sulfo, sulfamoyl, sulfonylamino, alkylaminosulfonyl or alkylsulfonylamino, wherein two vicinal residues R2, R3, R4, R5 and R6, together with the C-atoms of the phenyl ring to which they are bonded, can optionally form an oxygen-containing 5-, 6- or 7-membered ring; and
R7 is hydrogen, alkyl or phenylalkyl;
said compound being in the form of the free base, a physiologically acceptable salt thereof or an enantiomer or diastereomer thereof, with the proviso of exclusion of
(a) compounds wherein the heteroarene is a pyrazolo[1,5-a]pyridine that has no R1 substituent and carries as the sole substituent a group X as defined above wherein R2 is methoxy; R3, R4, R5, R6 and R7 are in each case hydrogen and
(i) Y is ethylene, n-propylene or n-butylene or
(ii) Y is n-pentylene and X is linked with the pyrazolo[1,5-a]pyridine core at the 2- or 3-position thereof; and
(b) the compound N-4-(4-(2-chlorophenyl)piperazin-1-yl)butyl-7-methylpyrazolo-[1,5-a]pyridin-3-ylcarbamide.

2. The compound of claim 1, having any of the formulas 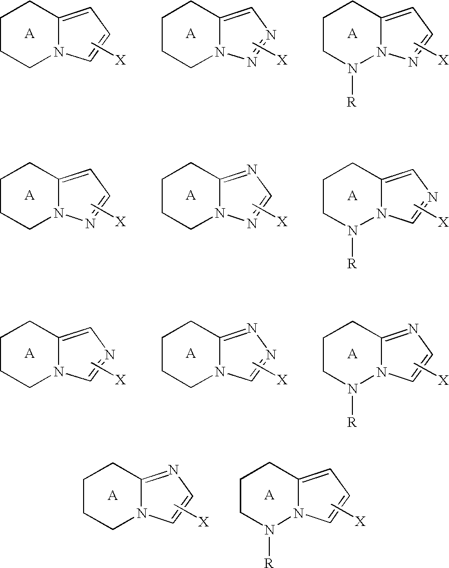
wherein;
the ring A is in each case saturated or aromatic;
the ring-forming C-atoms of rings A and B can optionally in each case and independently of each other be substituted with R1; and
R, R1 and X are as defined in claim 1.

3. The compound of claim 1 wherein Y is a group —(CH₂)ₙ— wherein n is 4 or 5.

4. The compound of claim 1 wherein R7 is hydrogen.

5. The compound of claim 1 that is an indolizine of general formula II 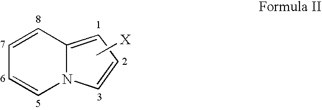
wherein:
the substituent X is linked to any position 1-3 and/or 5-8 of the indolizine and is a group of general formula X1 as defined in claim 1; and
the indolizine can optionally in positions 1-3 and/or 5-8, apart from X, also carry one or more additional substituents R1, said R1 substituents being independently hydroxy, alkyl, alkyloxy, alkylthio, alkenyl, alkynyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl or alkylsulfonylamino.

6. The compound of claim 5, wherein X is linked to the 1-, 2- or 3-position of the indolizine.

7. The compound of claim 5, wherein X represents is a group of general formula X2 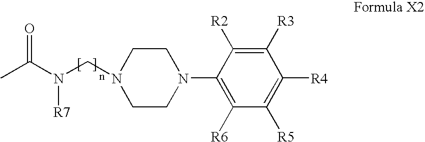
wherein:
n is 4 or 5; and
R2, R3, R4, R5, R6 and R7 are residues as described in claim as defined in claim 1.

8. The compound of claim 5, wherein R4 is hydrogen and (a) at least one of the two substituents R2 and R3 is a halogen atom or a methoxy group, or (b) R2 and R3 together with the phenyl residue to which they are bonded form a chromane or dihydrobenzofurane.

9. The compound of claim 1 that is a pyrazolo[1,5-a]pyridine of general formula III 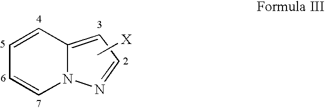
wherein:
the substituent X is linked to any position 2-7 of the pyrazolo[1,5-a]pyridine and is a group of general formula X1 as defined in claim 1; and
the pyrazolo[1,5-a]pyridine can optionally in positions 2-7, apart from X, also carry one or more additional substituents R1, said R1 substituents being independently hydroxy, alkyl, alkyloxy, alkylthio, alkenyl, alkynyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl or alkylsulfonylamino.

10. The compound of claim 9, wherein the X group is linked to positions 2, 5 or 6 of the pyrazolo[1,5-a]pyridine.

11. The compound of claim 9, wherein the pyrazolo[1,5-a]pyridine in position 5 carries a methoxy or trifluoromethyl residue and/or in position 6 a halogen atom.

12. The compound of claim 9, wherein X is a group of general formula X2 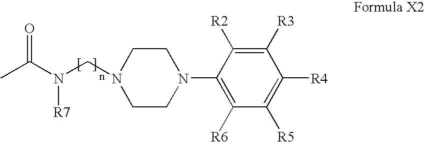
wherein:
n is 4 or 5; and
R2, R3, R4, R5, R6 and R7 are residues, as described in claim as defined in claim 1.

13. The compound of claim 9, wherein R4 is hydrogen and (a) the substituents R2 and R3 are independently halogen, alkyl, alkyloxy, phenylalkyloxy, alkylthio, trifluoromethyl, cyano or nitro, or the two substituents R2 and R3 together form a chromane or dihydrobenzofurane ring.

14. The compound of claim 1 that is a 4,5,6,7-tetrahydropyrazolo[1,5-a]pyridine of general formula IV 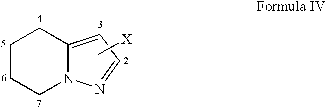
wherein:
the substituent X is linked to any position 2 or 3 of the 4,5,6,7-tetrahydropyrazolo[1,5-a]pyridine and represents is a group of general formula X1 as defined in claim 1; and
the 4,5,6,7-tetrahydropyrazolo[1,5-a]pyridine can optionally in positions 2-7 apart from the X group, also carry one or more additional substituents R1, said R1 substituents being independently hydroxy, alkyl, alkyloxy, alkylthio, alkenyl, alkynyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl or alkylsulfonylamino.

15. The compound of claim 13, wherein X is a group of general formula X2 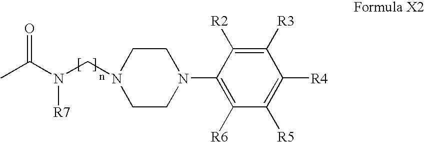
wherein:
n is 4 or 5; and
R2, R3, R4, R5, R6 and R7 are residues as defined in claim 1.

16. The compound of claim 14, wherein R4 is hydrogen and at least one of the substituents R2 and R3 is a halogen atom or a methoxy group.

17. The compound of claim 1 that is a 5,6,7,8-tetrahydroindolizine of general formula V 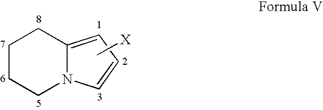
wherein:
the substituent X is linked to any position 1, 2 or 3 of the 5,6,7,8-tetrahydroindolizine and is a group of general formula X1 as defined in claim 1; and
the 5,6,7,8-tetrahydroindolizine can optionally in positions 1-3 and 5-8, apart from the X group, also carry one or more additional substituents R1, said R1 substituents being independently hydroxy, alkyl, alkyloxy, alkylthio, alkenyl, alkynyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl or alkylsulfonylamino.

18. The compound of claim 17, wherein X is a group of general formula X2 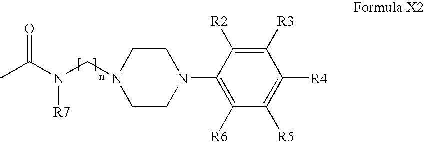
wherein:
n is 4 or 5; and
R2, R3, R4, R5, R6 and R7 are residues as defined in claim 1.

19. The compound of claim 17, wherein R4 is hydrogen and at least one of the substituents R2 and R3 is a halogen atom or a methoxy group.

20. The compound of claim 1 having general formula V1 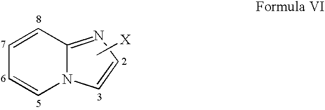
which wherein:
the substituent X is linked to any position 2-3 or 5-8 of the heteroarene core and is a group of general formula X1 as defined in claim 1; and
the heteroarene core of formula VI can optionally in positions 2-3 and 5-8, apart from the X group, also carry one or more additional substituents R1, said R1 substituents being independently hydroxy, alkyl, alkyloxy, alkylthio, alkenyl, alkynyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl or alkylsulfonylamino.

21. The compound of claim 20, wherein X is a group of general formula X2 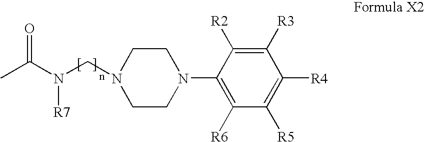
wherein;
n is 4 or 5; and
R2, R3, R4, R5, R6 and R7 are residues as defined in claim 1.

22. The compound of claim 20, wherein R4 is hydrogen and at least one of the substituents R2 and R3 is a halogen atom or a methoxy group.

23. The compound of claim 1 having general formula VII 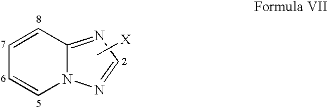
wherein:
the substituent X is linked to any position 2 or 5-8 of the heteroarene core and is a group of general formula X1 as defined in claim 1; and
the heteroarene core can optionally in positions 2 and 5-8, apart from the X group, also carry one or more additional substituents R1, said R1 substituents being independently hydroxy, alkyl, alkyloxy, alkylthio, alkenyl, alkynyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl or alkylsulfonylamino.

24. The compound of claim 23, wherein X is a group of general formula X2 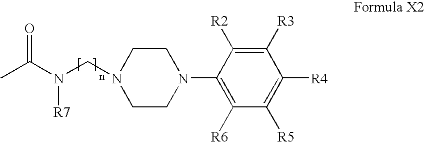
wherein:
n is 4 or 5; and
R2, R3, R4, R5, R6 and R7 are residues as defined in claim 1.

25. The compound of claim 23, wherein R4 is hydrogen and at least one of the substituents R2 and R3 is a halogen atom.

26. The compound of claim 1 having general formula VIII 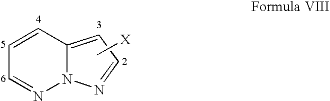
wherein:
the substituent X is linked to any position 26 of the heteroarene core and is a group of general formula X1 as defined in claim 1; and
the heteroarene core can optionally in positions 2-6, apart from the X group, also carry one or more additional substituents R1, said R1 substituents being independently hydroxy, alkyl, alkyloxy, alkylthio, alkenyl, alkynyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl or alkylsulfonylamino.

27. The compound of claim 26, wherein X is a group of general formula X2 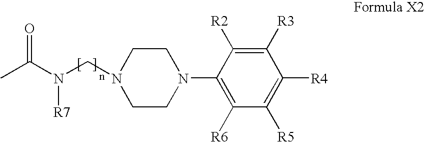
wherein:
n is 4 or 5; and
R2, R3, R4, R5, R6 and R7 are residues as defined in claim 1.

28. The compound of claim 26, wherein R4 is hydrogen and at least one of the substituents R2 and R3 is a halogen atom.

29. The compound of claim 1 having general formula IX 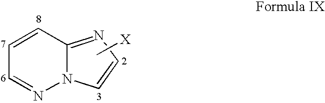
wherein:
the substituent X is linked to an position 2-3 or 6-8 of the heteroarene core and is a group of general formula X1 as defined in claim 1; and
the heteroarene core can optionally in positions 2-3 and/or 6-8, apart from the X group, also carry one or more additional substituents R1, said R1 substituents being independently hydroxy, alkyl, alkyloxy, alkylthio, alkenyl, alkynyl, phenyl, phenoxy, halogen, trifluoromethyl, alkylcarbonyl, phenylcarbonyl, alkyloxycarbonyl, cyano, nitro, amino, carboxy, sulfo, sulfamoyl, sulfonylamino, alkylaminosulfonyl or alkylsulfonylamino.

30. The compound of claim 29, wherein X is a group of general formula X2 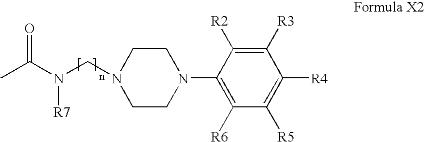
wherein:
n is 4 or 5 and
R2, R3, R4, R5, R6 and R7 are residues as defined in claim 1.

31. The compound of claim 29, wherein R4 is hydrogen and at least one of the substituents R2 and R3 is a methoxy residue or a halogen atom.

32. A compound selected from
N-4-(4-(2-methoxyphenyl)piperazin-1-yl)butylindolizin-1-ylcarbamide;
N-4-(4-(2-methoxyphenyl)piperazin-1-yl)butylindolizin-2-ylcarbamide;
N-4-(4-(3-chloro-2-methoxyphenyl)piperazin-1-yl)butylindolizin-2-ylcarbamide;
N-4-(4-(2,3-dichlorophenyl)piperazin-1-yl)butylindolizin-2-ylcarbamide;
N-4-(4-(2,3-difluorophenyl)piperazin-1-yl)butylindolizin-2-ylcarbamide;
N-4-(4-(2,3-dihydrobenzofuran-7-yl)piperazin-1-yl)butylindolizin-2-ylcarbamide;
N-4-(4-(chroman-8-yl)piperazin-1-yl)butylindolizin-2-ylcarbamide;
N-4-(4-(2-methoxyphenyl)piperazin-1-yl)butyl-5,6,7,8-tetrahydroindolizin-2-yl-carbamide;
N-4-(4-(2,3-dichlorophenyl)piperazin-1-yl)butyl-5,6,7,8-tetrahydroindolizin-2-yl-carbamide;
N-4-(4-(2-methoxyphenyl)piperazin-1-yl)butyl-1-cyano-2-methylindolizin-3-yl-carbamide;
N-2-(4-(2,3-dichlorophenyl)piperazin-1-yl)ethylpyrazolo[1,5-a]pyridin-2-yl-carbamide;
N-3-(4-(2,3-dichlorophenyl)piperazin-1-yl)propylpyrazolo[1,5-a]pyridin-2-ylcarbamide;
N-4-(4-phenylpiperazin-1-yl)butylpyrazolo[1,5-a]pyridin-2-ylcarbamide;
N-4-(4-(2-methylphenyl)piperazin-1-yl)butylpyrazolo[1,5-a]pyridin-2-yl-carbamide;
N-4-(4-(2-biphenyl)piperazin-1-yl)butylpyrazolo[1,5-a]pyridin-2-ylcarbamide;
N-4-(4-(2-ethoxyphenyl)piperazin-1-yl)butylpyrazolo[1,5-a]pyridin-2-yl-carbamide;
N-4-(4-(2-benzyloxyphenyl)piperazin-1-yl)butylpyrazolo[1,5-a]pyridin-2-yl-carbamide;
N-4-(4-(2-methylmercaptophenyl)piperazin-1-yl)butylpyrazolo[1,5-a]pyridin-2-yl-carbamide;
N-4-(4-(2-fluorophenyl)piperazin-1-yl)butylpyrazolo[1,5-a]pyridin-2-yl-carbamide;
N-4-(4-(2-trifluoromethylphenyl)piperazin-1-yl)butylpyrazolo[1,5-a]pyridin-2-yl-carbamide;
N-4-(4-(2-cyanophenyl)piperazin-1-yl)butylpyrazolo[1,5-a]pyridin-2-ylcarbamide;
N-4-(4-(2-nitrophenyl)piperazin-1-yl)butylpyrazolo[1,5-a]pyridin-2-ylcarbamide;
N-4-(4-(4-methoxyphenyl)piperazin-1-yl)butylpyrazolo[1,5-a]pyridin-2-yl-carbamide;
N-4-(4-(3-chloro-2-methoxyphenyl)piperazin-1-yl)butylpyrazolo[1,5-a]pyridin-2-ylcarbamide;
N-4-(4-(2,3-dichlorophenyl)piperazin-1-yl)butylpyrazolo[1,5-a]pyridin-2-yl-carbamide;
N-4-(4-(2,3-dimethylphenyl)piperazin-1-yl)butylpyrazolo[1,5-a]pyridin-2-ylcarbamide;
N-4-(4-(2,3-dihydrobenzofuran-7-yl)piperazin-1-yl)butylpyrazolo[1,5-a]pyridin-2-ylcarbamide;
N-4-(4-(chroman-8-yl)piperazin-1-yl)butylpyrazolo[1,5-a]pyridin-2-ylcarbamide;
N-4-(4-(2,4-dimethoxyphenyl)piperazin-1-yl)butylpyrazolo[1,5-a]pyridin-2-ylcarbamide;
N-4-(4-(2-methoxyphenyl)piperazin-1-yl)butyl-3-bromopyrazolo[1,5-a]pyridin-2-ylcarbamide;
N-4-(4-(2-methoxyphenyl)piperazin-1-yl)butyl-3-chloropyrazolo[1,5-a]pyridin-2-ylcarbamide;
N-4-(4-(2,3-dichlorophenyl)piperazin-1-yl)butyl-5-methoxypyrazolo[1,5-a]-pyridin-2-ylcarbamide;
N-4-(4-(2-methoxyphenyl)piperazin-1-yl)butyl-5-methoxypyrazolo[1,5-a]pyridin-2-ylcarbamide;
N-4-(4-(2-methoxyphenyl)piperazin-1-yl)butyl-5-methylpyrazolo[1,5-a]pyridin-2-ylcarbamide;
N-4-(4-(2,3-dichlorophenyl)piperazin-1-yl)butyl-5-trifluoromethylpyrazolo[1,5-a]-pyridin-2-ylcarbamide;
N-4-(4-(2-methoxyphenyl)piperazin-1-yl)butyl-5-trifluoromethylpyrazolo[1,5-a]-pyridin-2-ylcarbamide;
N-4-(4-(2,3-dichlorophenyl)piperazin-1-yl)butyl-6-bromopyrazolo[1,5-a]pyridin-2-ylcarbamide;
N-4-(4-(2-methoxyphenyl)piperazin-1-yl)butyl-6-bromopyrazolo[1,5-a]pyridin-2-ylcarbamide;
N-4-(4-(2,3-dichlorophenyl)piperazin-1-yl)butyl-6-chloropyrazolo[1,5-a]pyridin-2-ylcarbamide;
N-4-(4-(2-methoxyphenyl)piperazin-1-yl)butyl-6-chloropyrazolo[1,5-a]pyridin-2-ylcarbamide;
N-4-(4-(2,3-dichlorophenyl)piperazin-1-yl)butyl-6-fluoropyrazolo[1,5-a]pyridin-2-ylcarbamide;
N-4-(4-(2-methoxyphenyl)piperazin-1-yl)butyl-6-fluoropyrazolo[1,5-a]pyridin-2-ylcarbamide;
N-4-(4-(2-methoxyphenyl)piperazin-1-yl)butyl-3-methoxycarbonylpyrazolo-[1,5-a]pyridin-2-ylcarbamide;
N-4-(4-(2,3-dichlorophenyl)piperazin-1-yl)butyl-3-methoxycarbonylpyrazolo-[1,5-a]pyridin-2-ylcarbamide;
N-5-(4-(2,3-dichlorophenyl)piperazin-1-yl)pentylpyrazolo[1,5-a]pyridin-2-yl-carbamide;
N-5-(4-(2,3-dichlorophenyl)piperazin-1-yl)pentyl-5-methoxypyrazolo[1,5-a]-pyridin-2-ylcarbamide;
N-5-(4-(2-methoxyphenyl)piperazin-1-yl)pentyl-5-methoxypyrazolo[1,5-a]-pyridin-2-ylcarbamide;
N-5-(4-(2,3-dichlorophenyl)piperazin-1-yl)pentyl-5-trifluoromethylpyrazolo-[1,5-a]pyridin-2-ylcarbamide;
N-5-(4-(2-methoxyphenyl)piperazin-1-yl)pentyl-5-trifluoromethylpyrazolo-[1,5-a]pyridin-2-ylcarbamide;
N-5-(4-(2,3-dichlorophenyl)piperazin-1-yl)pentyl-6-bromopyrazolo[1,5-a]pyridin-2-ylcarbamide;
N-5-(4-(2-methoxyphenyl)piperazin-1-yl)pentyl-6-bromopyrazolo[1,5-a]pyridin-2-ylcarbamide;
N-5-(4-(2,3-dichlorophenyl)piperazin-1-yl)pentyl-6-chloropyrazolo[1,5-a]pyridin-2-ylcarbamide;
N-5-(4-(2-methoxyphenylpiperazin-1-yl)pentyl-6-chloropyrazolo[1,5-a]pyridin-2-ylcarbamide;
N-5-(4-(2,3-dichlorophenyl)piperazin-1-yl)pentyl-6-fluoropyrazolo[1,5-a]pyridin-2-ylcarbamide;
N-5-(4-(2-methoxyphenyl)piperazin-1-yl)pentyl-6-fluoropyrazolo[1,5-a]pyridin-2-ylcarbamide;
trans-N-(4-((4-(2-methoxyphenyl)piperazin-1-yl)methyl)cyclohex-1-yl)methyl-pyrazolo[1,5-a]pyridin-2-ylcarbamide;
trans-N-(4-((4-(2,3-dichlorophenyl)piperazin-1-yl)methyl)cyclohex-1-yl)methyl-pyrazolo[1,5-a]pyridin-2-ylcarbamide;
N-2-(4-(2,3-dichlorophenyl)piperazin-1-yl)ethylpyrazolo[1,5-a]pyridin-3-yl-carbamide;
N-3-(4-(2,3-dichlorophenyl)piperazin-1-yl)propylpyrazolo[1,5-a]pyridin-3-yl-carbamide;
N-4-(4-(2,3-dichlorophenyl)piperazin-1-yl)butylpyrazolo[1,5-a]pyridin-3-yl-carbamide;
N-5-(4-(2,3-dichlorophenyl)piperazin-1-yl)pentylpyrazolo[1,5-a]pyridin-3-yl-carbamide;
trans-N-(4-((4-(2-methoxyphenyl)piperazin-1-yl)methyl)cyclohex-1-yl)methyl-pyrazolo[1,5-a]pyridin-3-ylcarbamide;
trans-N-(4-((4-(2,3-dichlorophenyl)piperazin-1-yl)methyl)cyclohex-1-yl)methyl-pyrazolo[1,5-a]pyridin-3-ylcarbamide;
N-2-(4-(2,3-dichlorophenyl)piperazin-1-yl)ethylpyrazolo[1,5-a]pyridin-5-yl-carbamide;
N-3-(4-(2,3-dichlorophenyl)piperazin-1-yl)propylpyrazolo[1,5-a]pyridin-5-yl-carbamide;
N-4-(4-(2,3-dichlorophenyl)piperazin-1-yl)butylpyrazolo[1,5-a]pyridin-5-yl-carbamide;
N-5-(4-(2,3-dichlorophenyl)piperazin-1-yl)pentylpyrazolo[1,5-a]pyridin-5-yl-carbamide;
N-4-(4-(2-methoxyphenyl)piperazin-1-yl)butyl-3-bromopyrazolo[1,5-a]pyridin-5-ylcarbamide;
N-4-(4-(2-methoxyphenyl)piperazin-1-yl)butyl-3-chloropyrazolo[1,5-a]pyridin-5-ylcarbamide;
N-5-(4-(2-methoxyphenyl)piperazin-1-yl)pentylpyrazolo[1,5-a]pyridin-5-yl-carbamide;
N-2-(4-(2,3-dichlorophenyl)piperazin-1-yl)ethylpyrazolo[1,5-a]pyridin-6-yl-carbamide;
N-3-(4-(2,3-dichlorophenyl)piperazin-1-yl)propylpyrazolo[1,5-a]pyridin-6-yl-carbamide;
N-4-(4-(2,3-dichlorophenyl)piperazin-1-yl)butylpyrazolo[1,5-a]pyridin-6-yl-carbamide;
N-5-(4-(2,3-dichlorophenyl)piperazin-1-yl)pentylpyrazolo[1,5-a]pyridin-6-yl-carbamide;
N-2-(4-(2,3-dichlorophenyl)piperazin-1-yl)ethyl-4,5,6,7-tetrahydropyrazolo-[1,5-a]pyridin-2-ylcarbamide;
N-3-(4-(2,3-dichlorophenyl)piperazin-1-yl)propyl-4,5,6,7-tetrahydropyrazolo-[1,5-a]pyridin-2-ylcarbamide;
N-4-(4-(2,3-dichlorophenyl)piperazin-1-yl)butyl-4,5,6,7-tetrahydropyrazolo-[1,5-a]pyridin-2-ylcarbamide;
N-4-(4-(2-methoxyphenyl)piperazin-1-yl)butyl-4,5,6,7-tetrahydropyrazolo-[1,5-a]pyridin-2-ylcarbamide;
N-4-(4-(2-methoxyphenyl)piperazin-1-yl)butyl-5-methyl-4,5,6,7-tetrahydro-pyrazolo[1,5-a]pyridin-2-ylcarbamide;
N-5-(4-(2-methoxyphenyl)piperazin-1-yl)pentyl-4,5,6,7-tetrahydropyrazolo-[1,5-a]pyridin-2-ylcarbamide;
N-5-(4-(2,3-dichlorophenyl)piperazin-1-yl)pentyl-4,5,6,7-tetrahydropyrazolo-[1,5-a]pyridin-2-ylcarbamide;
N-2-(4-(2,3-dichlorophenyl)piperazin-1-yl)ethyl-4,5,6,7-tetrahydropyrazolo-[1,5-a]pyridin-3-ylcarbamide;
N-3-(4-(2,3-dichlorophenyl)piperazin-1-yl)propyl-4,5,6,7-tetrahydropyrazolo-[1,5-a]pyridin-3-ylcarbamide;
N-4-(4-(2,3-dichlorophenyl)piperazin-1-yl)butyl-4,5,6,7-tetrahydropyrazolo-[1,5-a]pyridin-3-ylcarbamide;
N-5-(4-(2,3-dichlorophenyl)piperazin-1-yl)pentyl-4,5,6,7-tetrahydropyrazolo-[1,5-a]pyridin-3-ylcarbamide;
N-4-(4-(2-methoxyphenyl)piperazin-1-yl)butyl-6-chloroimidazo[1,2-a]pyridin-2-ylcarbamide;
N-4-(4-(2,3-dichlorophenyl)piperazin-1-yl)butyl-6-chloroimidazo[1,2-a]pyridin-2-ylcarbamide;
N-4-(4-(2-methoxyphenyl)piperazin-1-yl)butyl-6-chloro-2-methylimidazo[1,2-a]-pyridin-3-ylcarbamide;
N-4-(4-(2-methoxyphenyl)piperazin-1-yl)butylimidazo[1,2-a]pyridin-6-yl-carbamide;
N-4-(4-(2,3-dichlorophenyl)piperazin-1-yl)butyl-1,2,4-triazolo[1,5-a]pyridin-2-yl-carbamide;
N-4-(4-(2,3-dichlorophenyl)piperazin-1-yl)butylpyrazolo[1,5-b]pyridazin-2-yl-carbamide;
N-4-(4-(2-methoxyphenyl)piperazin-1-yl)butyl-6-chloroimidazo[1,2-b]pyridazin-2-ylcarbamide; and
N-4-(4-(2-methoxyphenyl)piperazin-1-yl)butyl-6-chloro-2-phenylimidazo-[1,2-b]pyridazin-3-ylcarbamide.

33. (canceled)

34. A pharmaceutical composition comprising one or more of the compounds of claim 1 and a pharmaceutically acceptable adjuvant.

35. The method of claim 39, wherein the disease or disorder comprises a central nervous system illness.

36. The method of claim 39, wherein the disease or disorder comprises a urinary tract disorder.

37. (canceled)

38. The method of claim 39, wherein the disease or disorder comprises a schizophrenia, depressive disorder, L-dopa- and/or neuroleptic drug-induced motor disturbance, Parkinson's disease, Segawa syndrome, restless leg syndrome, hyperprolactinemia, hyperprolactinoma, attention deficit hyperactivity syndrome (ADHS) and/or urinary incontinence.

39. A method for treating a disease or disorder of the central nervous system or urinary tract in a mammal comprising administering to the mammal one or more compounds of claim 1.

40. The method of claim 39, wherein the disease or disorder comprises a pychosis, schizophrenia, anxiety disorder, compulsive disorder, drug dependency, depressive disorder, drug-induced extrapyramidal motor disturbance, Parkinson's disease, Segawa syndrome, Tourette's syndrome, restless leg syndrome, sleeping disorder, nausea, cognitive disorder, male erectile dysfunction, hyperprolactinemia, hyperprolactinoma, glaucoma, attention deficit hyperactive syndrome (ADHS), autism, stroke and/or urinary incontinence.

41. A process for preparing a compound of claim 1, comprising reacting an acid derivative A 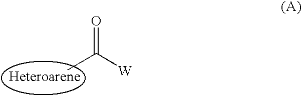
with a free base of general formula C 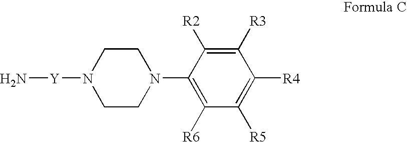
wherein:
(a) W is OH, Cl, Br or a group 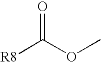
wherein R8 is alkyl;
(b) heteroarene is a group 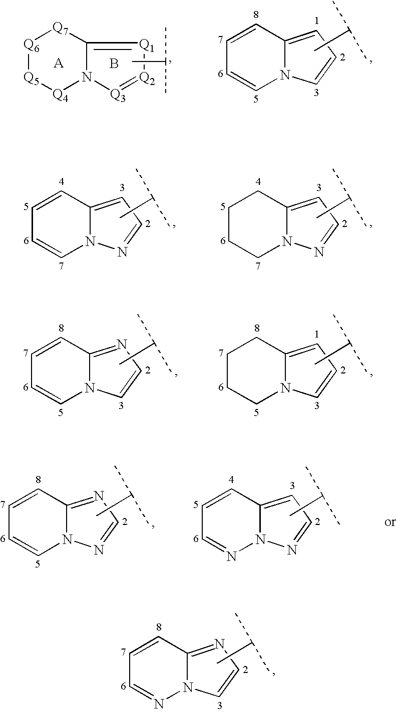
wherein
A, B, Q1, Q2, Q3, Q4, Q5, Q6 and Q7 are as defined in claim 1;
the crossed through bond for each of the heteroarenes stands for a bond of the —C(O)—W group to a ring-forming C-atom of an aromatic ring of the heteroarene; and
the heteroarene can optionally in each case carry one or more further substituents R1 or R, as defined in claim 1;
(c) Y, R2, R3, R4, R5 and R6 are as defined in claim 1, and
(d) if the substituent W is a hydroxy group, the acid derivative prior to reaction with the free base of general formula C is activated by addition of one or more activation reagents.

42. A process for preparing a carboxylic acid derivative of a pyrazolo[1,5-a]pyridine of general formula 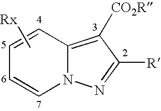
comprising conversion of a pyridine of formula 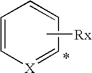
with O-(2,4-dinitrophenyl)hydroxylamine to an N-aminopyridine of formula 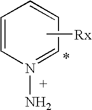
and subsequent cycloaddition reaction with a propiolic acid ester of formula 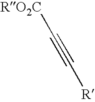
wherein Rx stands for 0, 1, 2, 3 or 4 identical or different halogen, alkyl, alkylcarbonyl, phenylcarbonyl, hydroxyalkyl, cyano, trifluoromethyl or alkyloxycarbonyl substituents, * denotes an unsubstituted CH group, R′ is hydrogen, alkyl, phenyl or alkyloxycarbonyl and R″ is alkyl.

43. A pharmaceutical composition comprising one or more of the compounds of claim 32 and a pharmaceutically acceptable adjuvant.

44. A method for treating a disease or disorder of the central nervous system or urinary tract in a mammal comprising administering to the mammal one or more compounds of claim 32.

45. The method of claim 44, wherein the disease or disorder comprises a central nervous system illness.

46. The method of claim 44, wherein the disease or disorder comprises a urinary tract disorder.

47. The method of claim 44, wherein the disease or disorder comprises a psychosis, schizophrenia, anxiety disorder, compulsive disorder, drug dependency, depressive disorder, drug-induced extrapyramidal motor disturbance, Parkinson's disease, Segawa syndrome, Tourette's syndrome, restless leg syndrome, sleeping disorder, nausea, cognitive disorder, male erectile dysfunction, hyperprolactinemia, hyperprolactinoma, glaucoma, attention deficit hyperactive syndrome (ADHS), autism, stroke and/or urinary incontinence.

48. The method of claim 44, wherein the disease or disorder comprises a schizophrenia, depressive disorder, L-dopa- and/or neuroleptic drug-induced motor disturbance, Parkinson's disease, Segawa syndrome, restless leg syndrome, hyperprolactinemia, hyperprolactinoma, attention deficit hyperactivity syndrome (ADHS) and/or urinary incontinence.
